(19) United States
(12) Patent Application Publication
(10) Pub. No.: US 2012/0046301 A1
(43) Pub. Date: Feb. 23, 2012

(54) Substituted Cyclic Carboxamide and Urea Derivatives as Ligands of the Vanilloid Receptor

(75) Inventors: Robert FRANK, Aachen (DE); Gregor Bahrenberg, Monschau-Konzen (DE); Thomas Christoph, Aachen (DE); Klaus Schiene, Juechen (DE); Jean De Vry, Herentals (BE)

(73) Assignee: Gruenenthal GmbH, Aachen (DE)

(21) Appl. No.: 13/213,400

(22) Filed: Aug. 19, 2011

Related U.S. Application Data
(60) Provisional application No. 61/375,332, filed on Aug. 20, 2010.

(30) Foreign Application Priority Data
Aug. 20, 2010 (EP) 10008723.8

(51) Int. Cl. A61K 31/496; A61K 31/415; C07D 401/14; C07D 401/04; C07D 231/12; A61P 29/00; A61P 25/06; A61P 25/24; A61P 25/28; A61P 25/00; A61P 25/08; A61P 11/00; A61P 13/00; A61P 13/02; A61P 1/00; A61P 27/02; A61P 17/00; A61P 31/22; A61P 1/12; A61P 3/00; A61P 19/02; A61P 19/10; A61P 25/30; A61P 25/32; A61P 9/00; A61P 17/02; A61P 15/00; A61P 25/16; A61P 11/06; A61P 11/08; A61P 3/04; A61K 31/4439

(52) U.S. Cl. 514/253.09; 514/254.05; 514/341; 514/406; 544/364; 544/366; 544/371; 546/275.4; 548/375.1

(57) ABSTRACT

Substituted cyclic carboxamide and urea compounds, a process for their preparation, pharmaceutical compositions containing these compounds, and the use of these compounds for the treatment and/or inhibition of pain and other conditions mediated by the vanilloid receptor 1.

### CROSS REFERENCE TO RELATED APPLICATIONS

This application claims priority from co-pending U.S. provisional patent application No. 61/375,332, filed Aug. 20, 2010, the entire disclosure of which is incorporated herein by reference.

### BACKGROUND OF THE INVENTION

The present invention relates to substituted cyclic carboxamide and urea derivatives, a process for their preparation, medicaments containing these compounds, as well as the use of these compounds for the production of medicaments.

The treatment of pain, in particular neuropathic pain, is of great importance in medicine. There is therefore a universal demand for effective pain treatments. The urgent demand for a patient-oriented and target-oriented treatment for chronic and non-chronic pain states, understood here to mean the successful and satisfactory treatment of pain for the patient, is also documented in the large number of scientific articles and reports that have appeared in recent times in the field of applied analgesics and basic research into nociception.

A suitable starting point for the treatment of pain, in particular pain selected from the group consisting of acute pain, chronic pain, neuropathic pain and visceral pain, particularly preferably neuropathic pain, is the vanilloid receptor of the subtype 1 (VR1/TRPV1), which is often also termed the capsaicin receptor. This receptor is stimulated inter alia by vanilloids such as e.g. capsaicin, heat and protons, and plays a central role in the development of pain. In addition it is important for a large number of further physiological and pathophysiological processes and represents a suitable target for the treatment of a large number of further diseases and illnesses, such as for example migraine, depression, neurodegenerative diseases, cognitive diseases, anxiety states, epilepsy, coughing, diarrhoea, pruritis, inflammations, disorders of the cardiovascular system, eating disorders, drug dependence, drug misuse and, in particular, urinary incontinence.

There is a need for further compounds having comparable or better properties, and not only in regard to the affinity for vanilloid receptors 1 (VR1/TRPV1 receptors) as such (potency, efficacy).

Thus, it can be advantageous to improve the metabolic stability, solubility in aqueous media or the permeability of the compounds. These factors can have a favourable effect on the oral bioavailability or can alter the PK/PD (pharmacokinetic/pharmacodynamic) profile, which can for example result in a more favourable action duration.

Also, a weak or non-existent interaction with transporter molecules that are involved in the uptake and elimination of medicinal substances should be regarded as an indication of an improved bioavailability and possibly minor medicament interactions. Furthermore, the interactions with enzymes involved in the breakdown and elimination of medicinal substances should be as few as possible, since such test results likewise indicate that possibly only minor medicament interactions or even none at all should be expected.

### SUMMARY OF THE INVENTION

An object of the invention was therefore to provide new compounds that have advantages compared to the compounds of the prior art. The compounds should in particular be suitable as pharmacological active substances, preferably in medicaments for the treatment and/or prevention of disorders or diseases that are mediated at least partly by vanilloid receptors 1 (VR1/TRPV1-receptors).

This object is achieved by the invention as described and claimed hereinafter.

It has now been surprisingly found that the substituted compounds of the general formula (I) shown hereinafter have an excellent affinity for the vanilloid receptor of subtype 1 (VR1/TRPV1 receptor) and are therefore suitable in particular for the prevention and/or treatment of disorders or diseases that are mediated at least in part by vanilloid receptors 1 (VR1/TRPV1). Likewise, the substituted compounds of the general formula (I) shown hereinafter have an anti-inflammatory activity.

A subject matter of the present invention are accordingly substituted compounds of the general formula (I),

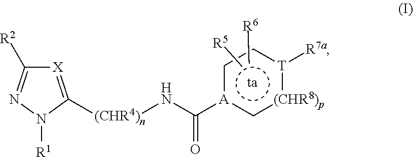

in which
X denotes CR³ or N,
   wherein R³ denotes H; C₁₋₁₀-alkyl, saturated or unsaturated, branched or unbranched, unsubstituted or monosubstituted or polysubstituted;
A denotes N, C or CH;
T denotes N, C or CR^{7b},
the symbol [symbol] denotes that the non-aromatic ring to can optionally include at least one unsaturated bond,
   with the proviso that when A denotes N, A is not part of an unsaturated bond, and
   with the proviso that when T denotes N, T is not part of an unsaturated bond,
p denotes 1, 2 or 3; preferably 1;
n denotes 0, 1, 2, 3 or 4, preferably ≠0; particularly preferably =1;
R⁰ denotes C₁₋₁₀-alkyl, saturated or unsaturated, branched or unbranched, unsubstituted or monosubstituted or polysubstituted; C₃₋₁₀-cycloalkyl or heterocyclyl, in each case saturated or unsaturated, unsubstituted or monosubstituted or polysubstituted; aryl or heteroaryl, in each case unsubstituted or monosubstituted or polysubstituted; C₃₋₁₀-cycloalkyl or heterocyclyl bridged via C₁₋₈-alkyl, in each case saturated or unsaturated, unsubstituted or monosubstituted or polysubstituted, wherein the alkyl chain can in each case be branched or unbranched, saturated, or unsaturated, unsubstituted, monosubstituted or polysubstituted; or aryl or heteroaryl bridged via C₁₋₈-alkyl, in each case unsubstituted or monosubstituted or polysubstituted, wherein the alkyl chain can in each case be branched or unbranched, saturated or unsaturated, unsubstituted, monosubstituted or polysubstituted;
R¹ denotes H; C₁₋₁₀-alkyl, saturated or unsaturated, branched or unbranched, unsubstituted or monosubstituted or polysubstituted; C₃₋₁₀-cycloalkyl¹ or heterocyclyl¹, in each case saturated or unsaturated, unsubstituted or monosubstituted or polysubstituted; aryl or heteroaryl, in each case unsubstituted or monosubstituted or polysubstituted; C₃₋₁₀-cycloalkyl¹ or heterocyclyl¹ bridged via C₁₋₈-alkyl, in each case saturated or unsaturated, unsubstituted or monosubstituted or polysubstituted, wherein the alkyl chain can in each case be branched or unbranched, saturated or unsaturated, unsubstituted, monosubstituted or polysubstituted; or aryl or heteroaryl bridged via C₁₋₈-alkyl, in each case unsubstituted or monosubstituted or polysubstituted, wherein the alkyl chain can in each case be branched or unbranched, saturated or unsaturated, unsubstituted, monosubstituted or polysubstituted; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰(S(═O)₂—R⁰; or SCl₃;
R² denotes H; R⁰; NO₂; CN; OH; SH; F; Cl; Br; I; CF₃; CF₂H; CFH₂; CF₂Cl; CFCl₂; CH₂CF₃; OCF₃; OCF₂H; OCFH₂; OCF₂Cl; OCFCl₂; SCF₃; SCF₂H; SCFH₂; SCF₂Cl; SCFCl₂; S(═O)₂—CF₃; S(═O)₂—CF₂H; S(═O)₂—CFH₂; or SF₅; preferably ≠H;
R⁴ denotes H; F; Cl; Br; I; OH; C₁₋₁₀-alkyl, saturated or unsaturated, branched or unbranched, unsubstituted or monosubstituted or polysubstituted;
R⁵, R⁶ and R⁸ denote in each case independently of one another H; F; Cl; Br; I; OH; OR⁰; or R⁰;
R^{7a} denotes R⁰; C(═O)—R⁰; C(═O)OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰)(S(═O)₂—R⁰;
R^{7b} denotes H; F; Cl; Br; I; or OH;
   with the proviso that R^{7a} cannot denote OH if T denotes CR^{7b} and R^{7b} denotes OH;
   with the proviso that R^{7a} cannot denote NH₂; NHR⁰; N(R⁰)₂; NH—S(═O)₂—R⁰; N(R⁰)(S(═O)₂—R⁰) if T denotes N;
      wherein “alkyl substituted”, “heterocyclyl substituted” and “cycloalkyl substituted” in the corresponding radicals denotes the substitution of one or more hydrogen atoms, in each case independently of one another, by F; Cl; Br; I; NO₂; CN; ═O; ═NH; ═C(NH₂)₂; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—W; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
      wherein “cycloalkyl¹ substituted” and “heterocyclyl¹ substituted” in the corresponding radicals denotes the substitution of one or more hydrogen atoms, in each case independently of one another, by F; Cl; Br; I; NO₂; CN; ═O; ═C(NH₂)₂; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
      wherein “aryl substituted” and “heteroaryl substituted” in the corresponding radicals denotes the substitution of one or more hydrogen atoms, in each case independently of one another, by F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
      in the form of the free compounds; the tautomers; the N-oxides; the racemate; the enantiomers, diastereomers, mixtures of the enantiomers or diastereomers or an individual enantiomer or diastereomer; or in the form of the salts of physiologcally compatible acids or bases; or optionally in the form of solvates.

The expressions “alkyl” and “C₁₋₁₀-alkyl”, “C₁₋₈-alkyl”, “C₁₋₈-alkyl”, “C₁₋₄-alkyl” include within the meaning of this invention acyclic saturated or unsaturated aliphatic hydrocarbon radicals, i.e. C₁₋₁₀-aliphatic radicals, C₁₋₈-aliphatic radicals, C₁₋₈-aliphatic radicals and C₁₋₆-aliphatic radicals, which may in each case be branched or unbranched and also unsubstituted or monosubstituted or polysubstituted, with respectively 1 to 10 or 1 to 8 or 1 to 6 or 1 to 4 carbon atoms, i.e. C₁₋₁₀-alkanyls, C₂₋₁₀-alkenyls and C₂₋₁₀-alkynyls, or C₁₋₈-alkanyls, C₂₋₈-alkenyls and C₂₋₈-alkynyls, or C₁₋₈-alkanyls, C₂₋₈-alkenyls and C₂₋₈-alkynyls, or C₁₋₄-alkanyls, C₂₋₄-alkenyls and C₂₋₄-alkynyls. In this connection alkenyls contain at least one C—C double bond and alkynyls at least one C—C triple bond. Preferably alkyl is selected from the group comprising methyl, ethyl, n-propyl, 2-propyl, n-butyl, iso-butyl, sec.-butyl, tert.-butyl, n-pentyl, iso-pentyl, neo-pentyl, n-hexyl, n-heptyl, n-octyl, n-nonyl, n-decyl, ethenyl (vinyl), ethynyl, propenyl (—CH₂CH═CH₂, —CH═CH—CH₃, —C(═CH₂)—CH₃), propynyl (—CH—C≡CH, —C≡C—CH₃), butenyl, butynyl, pentenyl, pentynyl, hexenyl and hexynyl, heptenyl, heptynyl, octenyl, octynyl, nonenyl, nonynyl, decenyl and decynyl.

The expressions “cycloalkyl” or “C₃₋₁₀-cycloalkyl” and “cycloalkyl¹” or “C₃₋₁₀-cycloalkyl¹” denote for the purposes of this invention cyclic aliphatic (cycloaliphatic) hydrocarbon radicals with 3, 4, 5, 6, 7, 8, 9 or 10 carbon atoms, i.e. C₃₋₁₀-cycloaliphatic radicals, in which the hydrocarbons may be saturated or unsaturated (but not aromatic), unsubstituted or monosubstituted or polysubstituted. The bonding of the cycloalkyl to the respective main general structure can take place via any arbitrary and possible ring member of the cycloaliphatic radical. The cycloalkyl residues may also be condensed with further saturated, (partially) unsaturated, (hetero)cyclic, aromatic or heteroaromatic ring system, i.e. with cycloalkyl, heterocyclyl, aryl or heteroaryl, which in turn may be unsubstituted or monosubstituted or polysubstituted. The cycloalkyl radicals may furthermore be singularly or multiply bridged, as for example in the case of adamantyl, bicyclo[2.2.1]heptyl or bicyclo[2.2.2]octyl. Preferably cycloalkyl is selected from the group comprising cyclopropyl, cyclobutyl, cyclopentyl, cyclohexyl, cycloheptyl, cyclooctyl, cyclononyl, cyclodecyl,

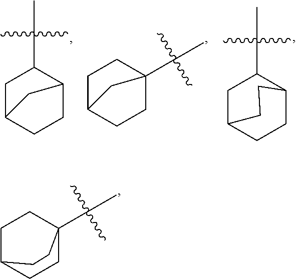

adamantyl,
cyclopentenyl, cyclohexenyl, cycloheptenyl and cyclooctenyl.

The expressions “heterocyclyl” or “heterocycloalkyl” and “heterocyclyl¹” or “heterocycloalkyl¹” include aliphatic saturated or unsaturated (but not aromatic) cycloalkyls with 3 to 10, i.e. 3, 4, 5, 6, 7, 8, 9 or 10 ring members, in which at least one, possibly also two or three carbon atoms are replaced by a hetero atom or a hetero atom group in each case independently selected from the group consisting of O, S, S(═O)₂, N, NH and N(C₁₋₈-alkyl), preferably N(CH₃), wherein the ring members may be unsubstituted or monosubstituted or polysubstituted. Heterocycles are thus heterocycloaliphatic radicals. The bonding of the heterocyclyl to the main general structure may take place via any arbitrary and possible ring member of the heterocyclyl radical. The heterocyclyl radicals may also be condensed with further saturated, (partially) unsaturated (hetero)cyclic or aromatic or heteroaromatic ring systems, i.e. with cycloalkyl, heterocyclyl, aryl or heteroaryl, which in turn may be unsubstituted or monosubstituted or polysubstituted. Preferred are heterocyclyl radicals from the group comprising azetidinyl, aziridinyl, azepanyl, azocanyl, diazepanyl, dithiolanyl, dihydroquinolinyl, dihydropyrrolyl, dioxanyl, dioxolanyl, dioxepanyl, dihydroindenyl dihydropyridinyl, dihydrofuranyl, dihydroisoquinolinyl, dihydroindolinyl, dihydroisoindolyl, imidazolidinyl, isoxazolidinyl, morpholinyl, oxiranyl, oxetanyl, pyrrolidinyl, piperazinyl, 4-methylpiperazinyl, piperidinyl, pyrazolidinyl, pyranyl, tetrahydropyrrolyl, tetrahydropyranyl, tetrahydroquinolinyl, tetrahydroisoquinolinyl, tetrahydroindolinyl, tetrahydrofuranyl, tetrahydropyridinyl, tetrahydrothiophenyl, tetrahydro-pyridoindolyl, tetrahydronaphthyl, tetrahydrocarbolinyl, tetrahydroisoxazolo-pyridinyl, thiazolidinyl and thiomorpholinyl.

The expression “aryl” denotes within the meaning of the present invention aromatic hydrocarbons with up to 14 ring members, including inter alia phenyls and naphthyls. Each aryl radical may be present unsubstituted or monosubstituted or polysubstituted, wherein the aryl substituents may be idential or different and may be in any arbitrary and possible position of the aryl. The bonding of the aryl to the main general structure may take place through any arbitrary and possible ring member of the aryl radical. The aryl radicals may also be condensed with further saturated, (partially) unsaturated, (hetero)cyclic, aromatic or heteroaromatic ring systems, i.e. with cycloalkyl, heterocyclyl, aryl or heteroaryl, which in turn may be unsubstituted or monosubstituted or polysubstituted. Examples of condensed aryl radicals are benzodioxolanyl and benzodioxanyl. Preferably aryl is selected from the group that contains phenyl, 1-naphthyl and 2-naphthyl which in each case may be unsubstituted or monosubstituted or polysubstituted. A particularly preferred aryl is phenyl, unsubstituted or monosubstituted or polysubstituted.

The expression “heteroaryl” denotes a 5- or 6-membered cyclic aromatic radical, which contains at least 1, or possibly also 2, 3, 4 or 5 hetero atoms, wherein hetero atoms can in each case be independently selected from the group S, N and O and the heteroaryl radical can be unsubstituted or monosubstituted or polysubstituted; in the case of substitution on the heteroaryl the substituents can be identical or different and can be in any arbitrary and possible position of the heteroaryl. The bonding to the main general structure can take place via any arbitrary and possible ring member of the heteroaryl radical. The heteroaryl can also be part of a bicyclic or polycyclic system with up to 14 ring members, in which the ring system can be formed with further saturated, (partially) unsaturated, (hetero)cyclic or aromatic or heteroaromatic rings, i.e. with cycloalkyl, heterocyclyl, aryl or heteroaryl, which in turn can be unsubstituted or monosubstituted or polysubstituted. It is preferred if the heteroaryl radical is selected from the group that includes benzofuranyl, benzoimidazolyl, benzothienyl, benzothiadiazolyl, benzothiazolyl, benzotriazolyl, benzooxazolyl, benzooxadiazolyl, quinazolinyl, quinoxalinyl, carbazolyl, quinolinyl, dibenzofuranyl, dibenzothienyl, furyl (furanyl), imidazolyl, imidazothiazolyl, indazolyl, indolizinyl, indolyl, isoquinolinyl, isoxazoyl, isothiazolyl, indolyl, naphthyridinyl, oxazolyl, oxadiazolyl, phenazinyl, phenothiazinyl, phtalazinyl, pyrazolyl, pyridyl (2-pyridyl, 3-pyridyl, 4-pyridyl), pyrrolyl, pyridazinyl, pyrimidinyl, pyrazinyl, purinyl, phenazinyl, thienyl (thiophenyl), triazolyl, tetrazolyl, thiazolyl, thiadiazolyl or triazinyl. Furyl, pyridyl and thienyl are particularly preferred.

The expressions “aryl, heteroaryl, heterocyclyl, cycloalkyl, heterocyclyl¹ or cycloalkyl¹ bridged via C₁₋₄-alkyl or C₁₋₈-alkyl” denote within the meaning of the invention that C₁₋₄-alkyl or C₁₋₈-alkyl and aryl or heteroaryl or heterocyclyl or cycloalkyl or heterocyclyl¹ or cycloalkyl¹ have the meanings defined above and the aryl or heteroaryl or heterocyclyl or cycloalkyl or heterocyclyl¹ or cycloalkyl¹ radical is bonded via a C₁₋₄-alkyl group or a C₁₋₈-alkyl group to the respective main general structure. The alkyl chain of the alkyl group can in all cases be branched or unbranched, unsubstituted or monosubstituted or polysubstituted. The alkyl chain of the alkyl group can furthermore in all cases be saturated or unsaturated, i.e. can be an alkylene, group, i.e. a C₁₋₄-alkylene group or a C₁₋₈-alkylene group, an alkenylene group, i.e. a C₂₋₄-alkenylene group or a C₂₋₈-alkenylene group, or an alkynylene group, i.e. a C₂₋₄-alkynylene group or a C₂₋₈-alkynylene group. Preferably C₁₋₄-alkyl is selected from the group comprising —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —C≡C—, —C≡C—CH₂—, —C≡C—CH₂—CH₂—, —C≡C—CH(CH₃)—, —CH₂—C≡C—CH₂— and —C═C—C═C— and C₁₋₈-alkyl is selected from the group comprising —CH₂—, —CH₂—CH₂—, —CH(CH₃)—, —CH₂—CH₂—CH₂—, —CH(CH₃)—CH₂—, —CH(CH₂CH₃)—, —CH₂—(CH₂)₂—CH₂—, —CH(CH₃)—CH₂—CH₂—, —CH₂—CH(CH₃)—CH₂—, —CH(CH₃)—CH(CH₃)—, —CH(CH₂CH₃)—CH₂—, —C(CH₃)₂—CH₂—, —CH(CH₂CH₂CH₃)—, —C(CH₃)(CH₂CH₃)—, —CH₂—(CH₂)₃—CH₂—, —CH(CH₃)—CH₂—CH₂—CH₂—, —CH₂—CH(CH₃)—CH₂—CH₂—, —CH(CH₃)—CH₂—CH(CH₃)—, —CH(CH₃)—CH(CH₃)—CH₂—, —C(CH₃)₂—CH₂—CH₂—, —CH₂—C(CH₃)₂—CH₂—, —CH(CH₂CH₃)—CH₂—CH₂—, —CH₂—CH(CH₂CH₃)—CH₂—, —C(CH₃)₂—CH(CH₃)—, —CH(CH₂CH₃)—CH(CH₃)—, —C(CH₃)(CH₂CH₃)—CH₂—, —CH(CH₂CH₂CH₃)—CH₂—, —C(CH₂CH₂CH₃)—CH₂—, —CH(CH₂CH₂CH₂CH₃)—, —C(CH₃)(CH₂CH₂CH₃)—, —C(CH₂CH₃)₂—, —CH₂—(CH₂)₄—CH₂—, —CH═CH—, —CH═CH—CH₂—, —C(CH₃)═CH₂—, —CH═CH—CH₂—CH₂—, —CH₂—CH═CH—CH₂—, —CH═CH—CH═CH—, —C(CH₃)═CH—CH₂—, —CH═C(CH₃)—CH₂—, —C(CH₃)═C(CH₃)—, —C(CH₂CH₃)═CH—, —CH═CH—CH₂—CH₂—CH₂—, —CH₂—CH═CH₂—CH₂—CH₂—, —CH═CH═CH—CH₂—CH₂—, —CH═CH₂—CH—CH═CH₂—, —C≡C—, —C≡C—CH₂—, —C≡C—CH₂—CH₂—, —C≡C—CH(CH₃)—, —CH₂—C≡C—CH₂—, —C≡C—C≡C—, —C≡C—C(CH₃)₂—, —C≡C—CH₂—CH₂—CH₂—, —CH₂—C≡C—CH₂—CH₂—, —C≡C—C═C—CH₂— and —C═C—CH₂—C═C—.

In connection with “alkyl”, “heterocyclyl” and “cycloalkyl” the expression “monosubstituted or polysubstituted” is understood within the meaning of the present invention to denote a monosubstitution or polysubstsitution, for example disubstitution, trisubstitution or tetrasubstitution of one or more hydrogen atoms in each case independently of one another by substituents selected from the group comprising F; Cl; Br; I; NO₂; CN; ═O; ═NH; ═C(NH₂)₂; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or)S(═O)₂N(R⁰)₂; wherein polysubstituted radicals are understood to mean those radicals that are polysubstituted, for example disubstituted, trisubstituted or tetrasubstituted, either on different or on the same atoms, for example trisubstituted on the same C atom as in the case of CF₃ or CH₂CF₃ or at different positions as in the case of CH(OH)—CH═CH—CHCl₂. A substituent can optionally in turn be monosubstituted or polysubstituted. The polysubstitution can take place with the same or with different substituents.

In connection with “cycloalkyl¹” and “heterocyclyl¹” the expression “monosubstituted or polysubstituted” is understood within the meaning of the present invention to denote monosubstitution or polysubstitution, for example disubstitution, trisubstitution or tetrasubstitution of one or more hydrogen atoms in each case independently of one another by substituents selected from the group comprising F; Cl; Br; I; NO₂; CN; ═O; ═C(NH₂)₂; CF₃; CF₂H; CFH₂; CF₂Cl; CFCl₂; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; SH; SCF₃; SCF₂H; SCFH₂; SCF₂Cl; SCFCl₂; SW; S(═O)R⁰; S(═O)₂R⁰; S(═O)₂OH; S(═O)₂OR⁰; S(═O)₂NH₂; S(═O)₂NHR⁰; or S(═O)₂N(R)₂;

wherein polysubstituted radicals are understood to denote those radicals that are polysubstituted, for example disubstituted, trisubstituted or tetrasubstituted either on different or on the same atoms, for example trisubstituted on the same C atom as in the case of 1,1-difluorocyclohexyl or at different positions as in the case of 1,2-difluorocyclohexyl. A substituent may optionally in turn be monosubstituted or polysubstituted. The polysubstitution may take place with the same or with different substituents.

Preferred “alkyl”, “heterocyclyl” and “cycloalkyl” substituents are selected from the group comprising F; Cl; Br; I; NO₂; CF₃; CN; ═O; ═NH; R⁰; C(═O)(R⁰ or H); C(═O)O(R⁰ or H); C(═O)N(R⁰ or H)₂; OH; OR⁰; O—C(═O)—R⁰; O—(C₁₋₈-Alkyl)-OH; O—(C₁₋₈-Alkyl)-O—C₁₋₈-Alkyl; OCF₃; N(R⁰ or H)₂; N(R⁰ or H)—C(═O)—W; N(R⁰ or H)—C(═O)—N(R⁰ or H)₂; SH; SCF₃; SW; S(═O)₂R⁰; S(═O)₂O(R⁰ or H) and S(═O)₂—N(R⁰ or H)₂.

Particularly preferred “alkyl”, “heterocyclyl” and “cycloalkyl” substituents are selected from the group consisting of: F; Cl; Br; I; NO₂; CF₃; CN; ═O; C₁₋₈-alkyl; aryl; heteroaryl; cycloalkyl; heterocyclyl; aryl, heteroaryl, C₃₋₁₀-cycloalkyl or heterocyclyl bridged via C₁₋₈-alkyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂, NH—C₁₋₈-alkyl; N(C₁₋₈-alkyl)₂; NH—C(═O)C₁₋₈-alkyl; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; and S(═O)₂—NH—C₁₋₈-heteroaryl.

Preferred “cycloalkyl¹” and “heterocyclyl¹” substituents are selected from the group consisting of F; Cl; Br; I; NO₂; CF₃; CN; ═O; R⁰; C(═O)(R⁰ or H); C(═O)O(R⁰ or H); C(═O)N(R⁰ or H)₂; OH; OR⁰; O—C(═O)—W; O—(C₁₋₈-alkyl)-OH; O—(C₁₋₈-alkyl)-O—C₁₋₈-alkyl; OCF₃; SH; SCF₃; SR⁰; S(═O)₂R⁰; S(═O)₂O(R⁰ or H) and S(═O)₂—N(R⁰ or H)₂.

Particularly preferred “cycloalkyl¹” and “heterocyclyl¹” substituents are selected from the group consisting of F; Cl; Br; I; NO₂; CF₃; CN; ═O; C₁₋₈-alkyl; aryl; heteroaryl; C₃₋₁₀-cycloalkyl; heterocyclyl; aryl, heteroaryl, C₃₋₁₀-cycloalkyl or heterocyclyl bridged via C₁₋₈-alkyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; and S(═O)₂—NH—C₁₋₈-heteroaryl.

In connection with “aryl” and “heteroaryl”, “monosubstituted or polysubstituted” is understood within the meaning of the present invention to denote monosubstitution or polysubstitution, for example disubstitution, trisubstitution or tetrasubstitution of one or more hydrogen atoms of the ring system in each case independently of one another by substituents selected from the group of F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or)S(═O)₂N(R⁰₂, on one or possibly different atoms, wherein a substituent can optionally in turn be monosubstituted or polysubstituted. The polysubstitution is carried out with the same or with different substituents.

Preferred “aryl” and “heteroaryl” substituents are F; Cl; Br; I; NO₂; CF₃; CN; R⁰; C(═O)(R⁰ or H); C(═O)O(R⁰ or H); C(═O)N(R⁰ or H)₂; OH; OR⁰; O—C(═O)—R⁰; C₁₋₈-alkyl; OCF₃; N(R⁰ or H)₂; N(R⁰ or H)—C(═O)—R⁰; N(R⁰ or H)—C(═O)—N(R⁰ or H)₂; SH; SCF₃; SR⁰; S(═O)₂R⁰; S(═O)₂O(R⁰ or H); S(═O)₂—N(R⁰ or H)₂.

Particularly preferred “aryl” and “heteroaryl” substituents are selected from the group consisting of F; Cl; Br; I; NO₂; CF₃; CN; C₁₋₈-alkyl; aryl; heteroaryl; C₃₋₁₀-cycloalkyl; heterocyclyl; aryl, heteroaryl, C₃₋₁₀-cycloalkyl or heterocyclyl bridged via C₁₋₈-alkyl; CHO; C(═O)C₁₋₈-alkyl; C(═O)aryl; C(═O)heteroaryl; CO₂H; C(═O)O—C₁₋₈-alkyl; C(═O)O-aryl; C(═O)O-heteroaryl; CONH₂; C(═O)NH—C₁₋₈-alkyl; C(═O)N(C₁₋₈-alkyl)₂; C(═O)NH-aryl; C(═O)N(aryl)₂; C(═O)NH-heteroaryl; C(═O)N(heteroaryl)₂; C(═O)N(C₁₋₈-alkyl)(aryl); C(═O)N(C₁₋₈-alkyl)(heteroaryl); C(═O)N(heteroaryl)(aryl); OH; O—C₁₋₈-alkyl; OCF₃; O—(C₁₋₈-alkyl)-OH; O—(C₁₋₈-alkyl)-O—C₁₋₈-alkyl; O-benzyl; O-aryl; O-heteroaryl; O—C(═O)C₁₋₈-alkyl; O—C(═O)aryl; O—C(═O)heteroaryl; NH₂, NH—C₁₋₈-alkyl; N(C₁₋₈-alkyl)₂; NH—C(═O)C₁₋₈-alkyl; NH—C(═O)-aryl; NH—C(═O)-heteroaryl; SH; S—C₁₋₈-alkyl; SCF₃; S-benzyl; S-aryl; S-heteroaryl; S(═O)₂C₁₋₈-alkyl; S(═O)₂aryl; S(═O)₂heteroaryl; S(═O)₂OH; S(═O)₂O—C₁₋₈-alkyl; S(═O)₂O-aryl; S(═O)₂O-heteroaryl; S(═O)₂—NH—C₁₋₈-alkyl; S(═O)₂—NH-aryl; S(═O)₂—NH—C₁₋₈-heteroaryl.

The compounds according to the invention are defined by substituents, for example by R¹, R² and R³ (substituents of the 1st generation), which in turn are optionally substituted (substituents of the 2nd generation). Depending on the definition these substituents of the substituents for their part may be resubstituted (substituents of the 3rd generation). If for example R¹=aryl (substituent of the 1st generation), then aryl can in turn be substituted, for example with C₁₋₈-alkyl (substituent of the 2nd generation). The result is the functional group aryl-C₁₋₈-alkyl. C₁₋₈-alkyl can in turn be resubstituted, for example with Cl (substituent of the 3rd generation). The result overall is then the functional group aryl-C₁₋₈-alkyl-Cl.

In a preferred embodiment the substituents of the 3rd generation may however not be resubstituted, i.e. there are then no substituents of the 4th generation.

In another preferred embodiment the substituents of the 2nd generation are not resubstituted, i.e. there are then already no substituents of the 3rd generation. In other words, in this embodiment, for example in the case of the general formula (I), the functional groups for R¹ to R⁸ can in each case optionally be substituted, and the respective constituents can then in their turn not be resubstituted however.

In some cases the compounds according to the invention are defined by substituents that form or carry an aryl or heteroaryl radical, in each case unsubstituted or monosubstituted or polysubstituted, or which together with the carbon atom(s) or hetero atom(s) as ring member or as ring members joining them form a ring, for example an aryl or heteroaryl, in each case unsubstituted or monosubstituted or polysubstituted. These aryl or heteroaryl radicals as well as the aromatic ring systems thereby formed can optionally be condensed with C₃₋₁₀-cycloalkyl or heterocyclyl, in each case saturated or unsaturated, or with aryl or heteroaryl, i.e. with a C₃₋₁₀-cycloalkyl such as cyclopentyl or with a heterocyclyl such as morpholinyl, or with an aryl such as phenyl or with a heteroaryl such as pyridyl, wherein the thereby condensed C₃₋₁₀-cycloalkyl or heterocyclyl radicals, aryl or heteroaryl radicals, can in turn in each case be unsubstituted or monosubstituted or polysubstituted.

In some cases the compounds according to the invention are defined by substituents that form or carry a C₃₋₁₀-cycloalkyl or heterocyclyl radical, in each case unsubstituted or monosubstituted or polysubstituted, or which together with the carbon atom(s) or hetero atom(s) as ring member or as ring members joining them form a ring, for example a C₃₋₁₀-cycloalkyl or heterocyclyl, in each case unsubstituted or monosubstituted or polysubstituted. These C₃₋₁₀-cycloalkyl or heterocyclyl radicals as well as the formed aliphatic ring systems can optionally be condensed with aryl or heteroaryl or with C₃₋₁₀-cycloalkyl or heterocyclyl, i.e. with an aryl such as phenyl or with a heteroaryl such as pyridyl or with a C₃₋₁₀-cycloalkyl such as cyclohexyl or with a heterocyclyl such as morpholinyl, wherein the thereby condensed aryl or heteroaryl radicals or C₃₋₁₀-cycloalkyl or heterocyclyl radicals can in turn in each case be unsubstituted or monosubstituted or polysubstituted.

Within the scope of the present invention the symbol

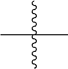

employed in formulae denotes a coupling of a corresponding radical to the respective main general structure.

The expression “(R⁰ or H)” within a radical means that R⁰ and H can be present in any possible combination within this radical. Thus, for example, the radical “N(R⁰ or H)₂” can denote “NH₂”, “NHR⁰” and “N(R⁰)₂”. If R⁰ as in the case of “N(R⁰)₂” is present more than once within a radical, then R⁰ can in each case have the same or different meanings: in the present example of “N(R⁰)₂”, R⁰ for example can denote aryl twice, thereby forming the functional group “N(aryl)₂”, or R⁰ can denote aryl once and denote C₁₋₁₀-alkyl once, thereby forming the functional group “N(aryl)(C₁₋₁₀-alkyl)”.

If a radical is present more than once within a molecule, such as for example the radical R⁰, then this radical can in each case have different meanings for different substituents: if for example R¹═R⁰ and also R²═R⁰, then when R⁰ is R¹ it can denote aryl, and when R⁰ is R² it can denote C₁₋₁₀-alkyl.

The expression salt formed with a physiologically compatible acid is understood within the meaning of the present invention to denote salts of the respective active substance with inorganic or organic acids that are physiologically compatible, especially when used in humans and/or mammals. The hydrochloride is particularly preferred. Examples of physiologically compatible acids are: hydrochloric acid, hydrobromic acid, sulfuric acid, methanesulfonic acid, p-toluenesulfonic acid, carbonic acid, formic acid, acetic acid, oxalic acid, succinic acid, tartaric acid, mandelic acid, fumaric acid, maleic acid, lactic acid, citric acid, glutamic, saccharic acid, monomethylsebacic acid, 5-oxo-proline, hexane-1-sulfonic acid, nicotinic acid, 2-, 3- or 4-aminobenzoic acid, 2,4,6-trimethylbenzoic acid, α-lipoic acid, acetylglycine, hippuric acid, phosphoric acid, aspartic acid. Citric acid and hydrochloric acid are particularly preferred.

Physiologically compatible salts with cations or bases are salts of the respective compound—as anion with at least one, preferably inorganic, cation, that are physiologically compatible, especially when used in humans and/or mammals. Particularly preferred are the salts of the alkali and alkaline earth metals, but also ammonium salts [NHₓR₄₋ₓ]⁺, wherein x=0, 1, 2, 3 or 4 and R denotes a branched or unbranched C₁₋₄-alkyl radical, in particular (mono) or (di)sodium, (mono) or (di)potassium, magnesium or calcium salts.

A further object of the present invention are compounds of the general formula (I)

in which
X denotes CR³ or N,
   In which R³ denotes H; or denotes C₁₋₁₀-alkyl, saturated or unsaturated, branched or unbranched, unsubstituted or monosubstituted or polysubstituted;
A denotes N, C or CH;
T denotes N, C or CR^{7b},
the symbol [symbol] denotes that the non-aromatic ring to can optionally include at least one unsaturated bond,
   with the proviso that when A denotes N, A is not part of an unsaturated bond, and
   with the proviso that when T denotes N, T is not part of an unsaturated bond,
p denotes 1, 2 or 3;
n denotes 1, 2, 3 or 4;
R⁰ denotes C₁₋₁₀-alkyl, saturated or unsaturated, branched or unbranched, unsubstituted or monosubstituted or polysubstituted; C₃₋₁₀-cycloalkyl or heterocyclyl, in each case saturated or unsaturated, unsubstituted or monosubstituted or polysubstituted; aryl or heteroaryl, in each case unsubstituted or monosubstituted or polysubstituted; C₃₋₁₀-cycloalkyl or heterocyclyl bridged via C₁₋₈-alkyl, in each case saturated or unsaturated, unsubstituted or monosubstituted or polysubstituted, wherein the alkyl chain can in each case be branched or unbranched, saturated or unsaturated, unsubstituted, monosubstituted or polysubstituted; or aryl or heteroaryl bridged via C₁₋₈-alkyl, in each case unsubstituted or monosubstituted or polysubstituted, wherein the alkyl chain can in each case be branched or unbranched, saturated or unsaturated, unsubstituted, monosubstituted or polysubstituted;
R¹ denotes H; C₁₋₁₀-alkyl, saturated or unsaturated, branched or unbranched, unsubstituted or monosubstituted or polysubstituted; C₃₋₁₀-cycloalkyl¹ or heterocyclyl¹, in each case saturated or unsaturated, unsubstituted or monosubstituted or polysubstituted; aryl or heteroaryl, in each case unsubstituted or monosubstituted or polysubstituted; C₃₋₁₀-cycloalkyl¹ or heterocyclyl¹ bridged via C₁₋₈-alkyl, in each case saturated or unsaturated, unsubstituted or monosubstituted or polysubstituted, wherein the alkyl chain can in each case be branched or unbranched, saturated or unsaturated, unsubstituted, monosubstituted or polysubstituted; or aryl or heteroaryl bridged via C₁₋₈-alkyl, in each case unsubstituted or monosubstituted or polysubstituted, wherein the alkyl chain can in each case be branched or unbranched, saturated or unsaturated, unsubstituted, monosubstituted or polysubstituted; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰)(S(═O)₂—R⁰; or SCl₃;
R² denotes H; R⁰; NO₂; CN; OH; SH; F; Cl; Br; I; CF₃; CF₂H; CFH₂; CF₂Cl; CFCl₂; CH₂CF₃; OCF₃; OCF₂H; OCFH₂; OCF₂Cl; OCFCl₂; SCF₃; SCF₂H; SCFH₂; SCF₂Cl; SCFCl₂; S(═O)₂—CF₃; S(═O)₂—CF₂H; S(═O)₂—CFH₂; or SF₅;
R⁴ denotes H;
R⁵, R⁶ and R⁸ in each case denote independently of one another H; F; Cl; Br; I; OH; OR⁰; or R⁰;
R^{7a} denotes R⁰; C(═O)—R⁰; C(═O)OH; C(═O)—OR⁰; C(═O)—NHR⁰; C(═O)—N(R⁰)₂; OH; O—R^{7c}; SH; S—R⁰; S(═O)₂—R⁰; S(═O)₂—OR⁰; S(═O)₂—NHR⁰; S(═O)₂—N(R⁰)₂; NH₂; NHR⁰; N(R)₂; NH—S(═O)₂—R⁰; or N(R⁰)(S(═O)₂—R⁰);
R^{7b} denotes H; F; Cl; Br; I; or OH;
   with the proviso that R^{7a} cannot denote OH if T denotes CR^{7b} and R^{7b} denotes OH;
   with the proviso that R^{7a} cannot denote NH₂; NHR⁰; N(R⁰)₂; NH—S(═O)₂—R⁰; N(R⁰)(S(═O)₂—R⁰; if T denotes N;
R^{7c} denotes C₁₋₁₀ alkyl, saturated or unsaturated, branched or unbranched, unsubstituted or monosubstituted or polysubstituted; aryl or heteroaryl;
   wherein “alkyl substituted”, “heterocyclyl substituted” and “cycloalkyl substituted” on the corresponding radicals denotes the substitution of one or more hydrogen atoms, in each case independently of one another, by F; Cl; Br; I; NO₂; CN; ═O; ═NH; ═C(NH₂)₂; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰); O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
   wherein “cycloalkyl¹ substituted” and “heterocyclyl¹ substituted” on the corresponding radicals denotes the substitution of one or more hydrogen atoms, in each case independently of one another, by F; Cl; Br; I; NO₂; CN; ═O; ═C(NH₂)₂; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—W; O—C(═O)—O—R⁰; O—(C═O)—NH—R⁰; O—C(═O)—) N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
   wherein “aryl substituted” and “heteroaryl substituted” on the corresponding radicals denotes the substitution of one or more hydrogen atoms, in each case independently of one another, by F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
   in the form of the free compounds; the tautomers; the N-oxides; the racemate; the enantiomers, diastereomers, mixtures of the enantiomers or diastereomers or an individual enantiomer or diastereomer; or in the form of the salts of physiologcally compatible acids or bases.

In preferred embodiments of the compounds according to the invention of the general formula (I) n denotes 1, 2, 3 or 4, preferably 1, 2 or 3, particularly preferably 1 or 2, and most particularly preferably 1.

Further preferred embodiments of the compounds according to the invention of the general formula (I) have the general formulae (Ia), (Ib), (Ic) or (Id):

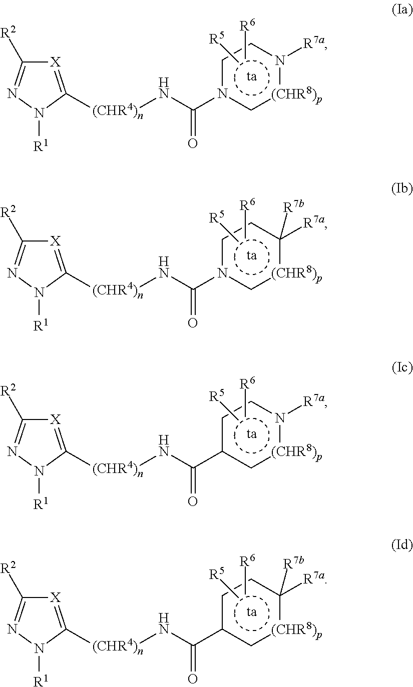

Most particularly preferred are compounds of the general formula (Ia).

The symbol [symbol] in the general formula (I) denotes that the non-aromatic ring ta can optionally have at least one, preferably just one, unsaturated bond, with the proviso that if A denotes N, A is not part of the unsaturated bond, and furthermore with the proviso that if T denotes N, then T is not part of the unsaturated bond. It is thus clear to the person skilled in the art that if a A denotes N, then A cannot form a double bond jointly with a carbon atom adjacent to A, i.e. a N═C-bond, and that if T denotes N, then T cannot form a double bond with a carbon atom adjacent to T, i.e. a N═C-bond. It is furthermore clear to the person skilled in the art that the atoms involved in the formation of a double bond such as a C═C bond or C═N bond in each case have one fewer substituent than if the same atoms jointly form a single bond such as a C—C— or C—N-bond. If the ring ta for example has an unsaturated bond between T and the adjacent carbon atom of the (CHR⁸)ₚ₌₁-group, then T denotes C (and not CR^{7b}) and the overall result is the formation of a C(R^{7a})═CR⁸-double bond, i.e. the substituent H is no longer present.

Further particularly preferred embodiments of the compounds according to the invention of the general formula (I) have the general formulae (Ia-1), (Ib-1), (Ic-1) (Id-1), (Ib-2), (Ib-3), (Ic-2), (Ic-3), (Id-2), (Id-3) and (Id-4):

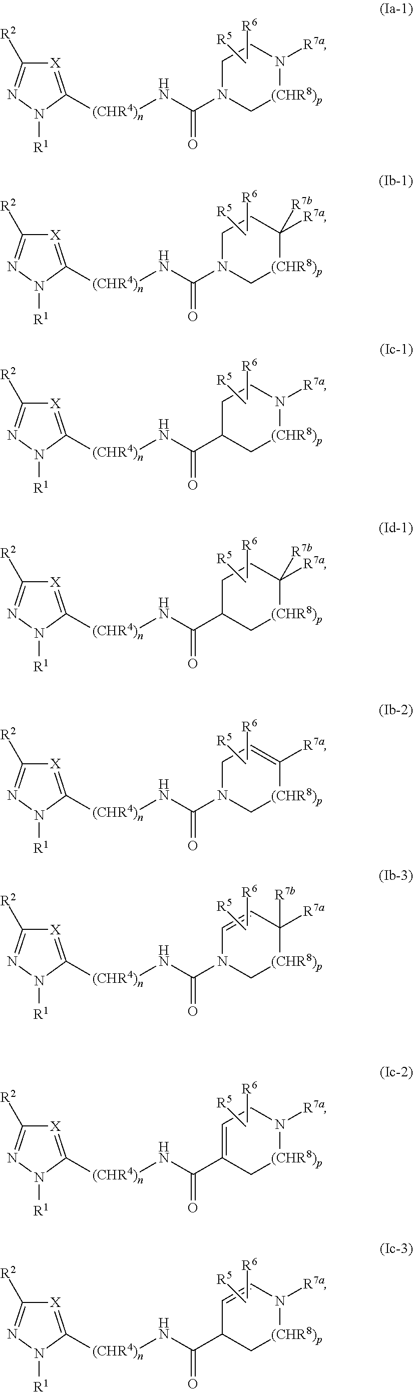

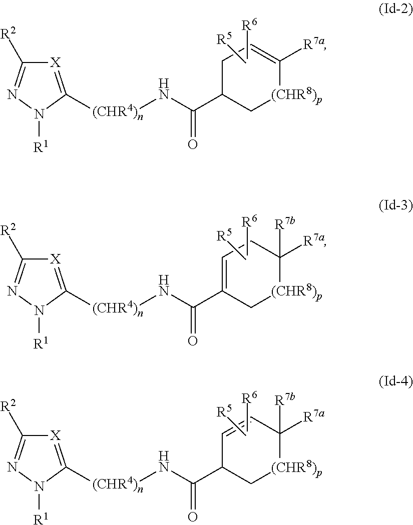

Particularly preferred are compounds of the general formula (Ia-1).

In a particularly preferred embodiment of the compounds according to the invention of the general formula (I) the radical R¹≠H.

In a further preferred embodiment of the compounds according to the invention of the general formula (I) the radical
R¹ denotes H; C₁₋₁₀-alkyl, C(═O)—C₁₋₁₀-alkyl, C(═O)—NH—C₁₋₁₀-alkyl, C(═O)—N(C₁₋₁₀-alkyl)₂, O—C₁₋₁₀-alkyl, S—C₁₋₁₀-alkyl, NH(C₁₋₁₀-alkyl), N(C₁₋₁₀-alkyl)₂, NH—S(═O)₂—C₁₋₁₀-alkyl, N(C₁₋₁₀-alkyl)-S(═O)₂—C₁₋₁₀-alkyl, S(═O)₂—C₁₋₁₀-alkyl, S(═O)₂—NH—C₁₋₁₀-alkyl, S(═O)₂—N(C₁₋₁₀-alkyl)₂, wherein C₁₋₁₀-alkyl can in each case be saturated or unsaturated, branched or unbranched, unsubstituted or monosubstituted or polysubstituted, with one or more substituents selected in each case independently of one another from the group consisting of F, Cl, Br, I, NO₂, CN, OH, O—C₁₋₄-alkyl, OCF₃, CF₃, NH₂, NH(C₁₋₄-alkyl), N(C₁₋₄-alkyl)₂, SH, S—C₁₋₄-alkyl, SCF₃, phenyl and pyridyl, wherein phenyl or pyridyl are in each case unsubstituted or monosubstituted or polysubstituted with one or more substituents in each case independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH;
   or denotes C₃₋₁₀-cycloalkyl¹ or heterocyclyl¹, in each case saturated or unsaturated, unsubstituted or monosubstituted or polysubstituted with one or more substituents in each case independently selected from the group consisting of F, Cl, Br, I, NO₂, CN, OH, ═O, O—C₁₋₄-alkyl, C₁₋₄-alkyl, OCF₃, CF₃, SH, S—C₁₋₄-alkyl, SCF₃, phenyl and pyridyl, wherein phenyl or pyridyl are in each case unsubstituted or monosubstituted or polysubstituted with one or more substituents in each case independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH;
   or denotes C₃₋₁₀-cycloalkyl¹ or heterocyclyl¹ bridged via C₁₋₈-alkyl, in each case saturated or unsaturated, unsubstituted or monosubstituted or polysubstituted with one or more substituents in each case independently selected from the group consisting of F, Cl, Br, I, NO₂, CN, OH, ═O, O—C₁₋₄-alkyl, C₁₋₄-alkyl, OCF₃, CF₃, SH, S—C₁₋₄-alkyl, SCF₃, phenyl and pyridyl, wherein phenyl or pyridyl are in each case unsubstituted or monosubstituted or polysubstituted with one or more substituents in each case independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; wherein the alkyl chain can in each case be branched or unbranched, saturated or unsaturated, unsubstituted or monosubstituted or polysubstituted with one or more substituents in each case independently selected from the group consisting of F, Cl, Br; I, OH and O—C₁₋₄-alkyl;
   or denotes C(═O)—C₃₋₁₀-cycloalkyl, O—C₃₋₁₀-cycloalkyl, S—C₃₋₁₀-cycloalkyl, in each case saturated or unsaturated, unsubstituted or monosubstituted or polysubstituted with one or more substituents in each case independently selected from the group consisting of F, Cl, Br, I, NO₂, CN, OH, ═O, O—C₁₋₄-alkyl, OCF₃, CF₃, NH₂, NH(C₁₋₄-alkyl), N(C₁₋₄-alkyl)₂, SH, S—C₁₋₄-alkyl, SCF₃, C₁₋₄-alkyl, phenyl and pyridyl, wherein phenyl or pyridyl are in each case unsubstituted or monosubstituted or polysubstituted with one or more substituents in each case independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH;
   or denotes aryl, heteroaryl, C(═O)-aryl, C(═O)-heteroaryl, O-aryl, O-heteroaryl, NH(aryl), N(aryl)₂, NH(heteroaryl), N(heteroaryl)₂, S(═O)₂-aryl, S(═O)₂-heteroaryl or aryl or heteroaryl bridged via C₁₋₈-alkyl, which in each case can be unsubstituted or monosubstituted or polysubstituted with one or more substituents in each case selected independently of one another from the group consisting of F, Cl, Br, I, NO₂, CN, OH, ═O, O—C₁₋₄-alkyl, C₁₋₄-alkyl, C₁₋₄-alkyl monosubstituted or disubstituted with OH, or OCF₃, CF₃, NH₂, NH(C₁₋₄-alkyl), N(C₁₋₄-alkyl)₂, SH, S—C₁₋₄-alkyl, SCF₃, S(═O)₂OH and NH—S(═O)₂—C₁₋₄-alkyl, and wherein optionally the alkyl chain may in each case be branched or unbranched, saturated or unsaturated, unsubstituted, monosubstituted or polysubstituted with one or more substituents in each case selected independently of one another from the group consisting of F, Cl, Br, I, OH and O—C₁₋₄-alkyl.

In another preferred embodiment of the compounds according to the invention of the general formula (I) the radical
R¹ denotes the partial structure (T1)

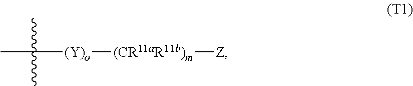

in which
Y denotes C(═O), O, S, S(═O)₂ or NR¹²,
   wherein R¹² denotes H; C₁₋₈-alkyl or S(═O)₂—C₁₋₈-alkyl, in which C₁₋₈-alkyl can in each case be saturated or unsaturated, branched or unbranched, unsubstituted or monosubstituted or polysubstituted with one or more substituents in each case selected independently of one another from the group consisting of F, Cl, Br, I, OH, O—C₁₋₄-alkyl, OCF₃, NH₂, NH—C₁₋₄-alkyl and N(C₁₋₄-alkyl)₂;
o denotes 0 or 1,
R^{11a} and R^{11b} in each denote independently of one another H; F; Cl; Br; I; NO₂; CF₃; CN; OH; OCF₃; NH₂; C₁₋₄-alkyl, O—C₁₋₄-alkyl, NH—C₁₋₄-alkyl, N(C₁₋₄-alkyl)₂, wherein C₁₋₄-alkyl can in each case be saturated or unsaturated, branched or unbranched, unsubstituted or monosubstituted or polysubstituted with one or more substituents in each case selected independently of one another from the group consisting of F, Cl, Br, I, O—C₁₋₄-alkyl, OH and OCF₃;
with the proviso that if R^{11a} and R^{11b} are bonded to the same carbon atom, only one of the substituents R^{11a} and R^{11b} can denote OH, OCF₃, NH₂, O—C₁₋₄-alkyl, NH—C₁₋₄-alkyl or N(C₁₋₄-alkyl)₂;
m denotes 0, 1, 2, 3 or 4;
Z denotes C₁₋₄-alkyl, saturated or unsaturated, branched or unbranched, unsubstituted or monosubstituted or polysubstituted with one or more substituents in each case selected independently of one another from the group consisting of F, Cl, Br, I, NO₂, CN, OH, ═O, O—C₁₋₄-alkyl, OCF₃, C(═O)—OH, CF₃, NH₂, NH(C₁₋₄-Alkyl), N(C₁₋₄-alkyl)₂, SH, S—C₁₋₄-alkyl, SCF₃ and S(═O)₂OH; C₃₋₁₀-cycloalkyl¹ or heterocyclyl¹, in each case saturated or unsaturated, unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, C(═O)—OH, CF₃, SH, S—C₁₋₄-alkyl, SCF₃, S(═O)₂OH, benzyl, phenyl, pyridyl and thienyl, wherein benzyl, phenyl, pyridyl, thienyl can in each case be unsubstituted or monosubstituted or polysubstituted with one or more substituents selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; aryl or heteroaryl, in each case unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, C₁₋₄-alkyl monosubstituted or disubstituted with OH, C(═O)—OH, CF₃, NH₂, NH(C₁₋₄-alkyl), N(C₁₋₄-alkyl)₂, SH, S—C₁₋₄-alkyl, SCF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents selected independently of one another from the group consisting of F, Cl, Br, I, NO₂, CN, OH, O—C₁₋₈-alkyl, OCF₃, C₁₋₄-alkyl, C(═O)—OH, CF₃, NH₂, NH(C₁₋₄-alkyl), N(C₁₋₄-Alkyl)₂, SH, S—C₁₋₄-alkyl, SCF₃ and S(═O)₂OH.

If m≠0, then the radicals R^{11a} and R^{11b} having regard to the aforementioned proviso can denote, on the same carbon atom as well as on different carbon atoms, in each case independently of one another H; F; Cl; Br; I; NO₂; CF₃; CN; OH; OCF₃; NH₂; C₁₋₄-alkyl, O—C₁₋₄-alkyl, NH—C₁₋₄-alkyl, N(C₁₋₄-alkyl)₂, wherein C₁₋₄-alkyl can in each case be saturated or unsaturated, branched or unbranched, unsubstituted or monosubstituted or polysubstituted with one or more substituents in each case selected independently of one another from the group consisting of F, Cl, Br, I, O—C₁₋₄-alkyl, OH and OCF₃.

Preferably the radical
R¹ denotes the partial structure (T1), in which
Y denotes C(═O), O, S, S(═O)₂ or NR¹²,
in which R¹² denotes H; methyl; ethyl; n-propyl; iso-propyl; n-butyl; sec.-butyl; tert.-butyl; S(═O)₂-methyl; S(═O)₂-ethyl;
o denotes 0 or 1;
R^{11a} and R^{11b} in each case denote independently of one another H; F; Cl; Br; I; NO₂; CF₃; CN; methyl; ethyl; n-propyl; iso-propyl; n-butyl; sec.-butyl; tert.-butyl; CH₂CF₃; OH; O-methyl; O-ethyl; O—(CH₂)₂—O—CH₃; O—(CH₂)₂—OH; OCF₃; NH₂; NH-methyl; N(methyl)₂; NH-ethyl; N(ethyl)₂; or N(methyl)(ethyl);
with the proviso that if R^{11a} and R^{11b} are bonded to the same carbon atom, only one of the substituents R^{11a} and R^{11b} can denote OH; OCF₃; O-methyl; O-ethyl; O—(CH₂)₂—O—CH₃; O—(CH₂)₂—OH; NH₂; NH-methyl; N(methyl)₂; NH-ethyl; N(ethyl)₂; or N(methyl)(ethyl);
m denotes 0, 1 or 2;
Z denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, ═O, O—C₁₋₄-alkyl, OCF₃, C(═O)—OH and CF₃; phenyl, naphthyl, furyl, pyridyl or thienyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₈-alkyl, SCF₃, C₁₋₄-alkyl monosubstituted or disubstituted with OH, benzyl and phenyl, wherein benzyl and phenyl can in each case be unsubstituted or monosubstituted or polysubstituted with one or more substituents selected independently of one another from the group consisting of F, Cl, Br, I, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₄-alkyl and SCF₃; C₃₋₁₀-cycloalkyl¹ or heterocyclyl¹, in each case saturated or unsaturated, unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃, benzyl, phenyl and pyridyl, wherein benzyl, phenyl and pyridyl can in each case be unsubstituted or monosubstituted or polysubstituted with one or more substituents selected independently of one another from the group consisting of F, Cl, Br, I, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₄-alkyl and SCF₃;
if m≠0, then the radicals are R^{11a} and R^{11b}, having regard to the aforementioned proviso, can on the same carbon atom as well as on different carbon atoms in each case denote independently of one another H; F; Cl; Br; I; NO₂; CF₃; CN; methyl; ethyl; n-propyl; iso-propyl; n-butyl; sec.-butyl; tert.-butyl; CH₂CF₃; OH; O-methyl; O-ethyl; O—(OH₂)₂—O—OH₃; O—(CH₂)₂—OH; OCF₃; NH₂; NH-methyl; N(methyl)₂; NH-ethyl; N(ethyl)₂; or N(methyl)(ethyl).

Particularly preferably the radical
R¹ denotes the partial structure (T1), in which
Y denotes C(═O), O, S, S(═O)₂ or NR¹²,
   wherein R¹² denotes H; methyl; ethyl; n-propyl; iso-propyl; n-butyl; sec.-butyl; tert.-butyl; S(═O)₂-methyl; S(═O)₂-ethyl;
o denotes 0 or 1;
R^{11a} and R^{11b} in each case denote independently of one another H; F; Cl; Br; I; methyl; ethyl; n-propyl; iso-propyl; n-butyl; sec.-butyl; tert.-butyl; OH; O-methyl; O-ethyl;
   with the proviso that if R^{11a} and R^{11b} are bonded to the same carbon atom, only one of the substituents R^{11a} and R^{11b} can denote OH; O-methyl; O-ethyl;
m denotes 0, 1 or 2;
Z denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-alkyl, OCF₃, and CF₃; C₃₋₁₀-cycloalkyl¹, saturated or unsaturated, unsubstituted or monosubstituted or polysubstituted with one or more substituents in each case selected independently of one another from the group consisting of F, Cl, Br, I, OH, O—C₁₋₄-alkyl, OCF₃, C₁₋₄-alkyl, CF₃, benzyl and phenyl, wherein benzyl and phenyl can in each case be unsubstituted or monosubstituted or polysubstituted with one or more substituents selected independently of one another from the group consisting of F, Cl, Br, I, OH, O—C₁₋₄-alkyl, OCF₃, C₁₋₄-alkyl, CF₃, and SCF₃; morpholinyl, thiomorpholinyl, piperidinyl, pyrrolidinyl, 4-methylpiperazinyl, piperazinyl, in each case unsubstituted or monosubstituted or polysubstituted with one or more substituents in each case selected independently of one another from the group consisting of F, Cl, Br, I, OH, O—C₁₋₄-alkyl, OCF₃, C₁₋₄-alkyl, CF₃, benzyl and phenyl, wherein benzyl and phenyl can in each case be unsubstituted or monosubstituted or polysubstituted with one or more substituents selected independently of one another from the group consisting of F, Cl, Br, I, OH, O—C₁₋₄-alkyl, OCF₃, C₁₋₄-alkyl, CF₃ and SCF₃; phenyl, naphthyl, pyridyl or thienyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃, SH, S—C₁₋₄-alkyl, C₁₋₄-alkyl monosubstituted or disubstituted with OH, SCF₃, benzyl and phenyl, wherein benzyl and phenyl can in each case be unsubstituted or monosubstituted or polysubstituted with one or more substituents selected independently of one another from the group consisting of F, Cl, Br, I, OH, O—C₁₋₄-alkyl, OCF₃, C₁₋₄-alkyl, CF₃ and SCF₃.

If m≠0, then the radicals are R^{11a} and R^{11b} can, having regard to the aforementioned proviso, on the same carbon atom as well as on different carbon atoms denote in each case independently of one another H; F; Cl; Br; I; methyl; ethyl; n-propyl; iso-propyl; n-butyl; sec.-butyl; tert.-butyl; OH; O-methyl; O-ethyl.

Most particularly preferably the radical
R¹ denotes the partial structure (T1),

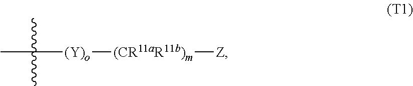

in which
Y denotes C(═O), O, S, S(═O)₂ or NR¹², preferably denotes C(═O) or S(═O)₂,
   wherein R¹² denotes H; methyl; ethyl; n-propyl; iso-propyl; n-butyl; sec.-butyl; tert.-butyl; S(═O)₂-methyl;
o denotes 0 or 1;
R^{11a} and R^{11b} in each case denote independently of one another H; methyl; ethyl; n-propyl; iso-propyl; n-butyl; sec.-butyl; tert.-butyl;
m denotes 0, 1 or 2;
Z denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-alkyl; C₃₋₁₀-cycloalkyl¹, saturated or unsaturated, morpholinyl, piperidinyl, 4-methylpiperazinyl, piperazinyl, in each case unsubstituted or monosubstituted or polysubstituted with one or more substituents in each case selected independently of one another from the group consisting of F, Cl, Br, I, OH, O—C₁₋₄-alkyl and C₁₋₄-alkyl; phenyl or pyridyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, C₁₋₄-alkyl monosubstituted or disubstituted with OH, CF₃, SH, S—C₁₋₄-Alkyl, SCF₃.

If m≠0, then the radicals R^{11a} and R^{11b} can on the same carbon atom as well as on different carbon atoms in each case denote independently of one another H; methyl; ethyl; n-propyl; iso-propyl; n-butyl; sec.-butyl; tert.-butyl.

Particularly preferably
R¹ denotes the partial structure (T1-1)

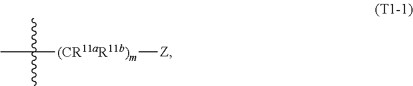

in which
R^{11a} and R^{11b} in each case denote independently of one another H; methyl; ethyl; n-propyl; iso-propyl; n-butyl; sec.-butyl; tert.-butyl;
m denotes 0, 1 or 2;
Z denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-alkyl; C₃₋₁₀-cycloalkyl¹, saturated or unsaturated, morpholinyl, piperidinyl, 4-methylpiperazinyl, piperazinyl, in each case unsubstituted or monosubstituted or polysubstituted with one or more substituents in each case selected independently of one another from the group consisting of F, Cl, Br, I, OH, O—C₁₋₄-alkyl and C₁₋₄-alkyl; phenyl or pyridyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, C₁₋₄-alkyl monosubstituted or disubstituted with OH, CF₃, SH, S—C₁₋₄-alkyl, SCF₃.

If m≠0, then the radicals R^{11a} and R^{11b} can on the same carbon atom as well as on different carbon atoms in each case denote independently of one another H; methyl; ethyl; n-propyl; iso-propyl; n-butyl; sec.-butyl; tert.-butyl.

In a further preferred embodiment of the compounds according to the invention of the general formula (I) the radical
R² denotes H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; C₁₋₁₀-alkyl, saturated or unsaturated, branched or unbranched, unsubstituted or monosubstituted or polysubstituted with one or more substituents in each case selected independently of one another from the group consisting of F, Cl, Br, I, NO₂, CN, OH, ═O, O—C₁₋₄-alkyl, OCF₃, C(═O)—OH, CF₃, NH₂, NH(C₁₋₄-alkyl), N(C₁₋₄-alkyl)₂, SH, S—C₁₋₄-alkyl, SCF₃ S(═O)₂OH, benzyl, phenyl, pyridyl and thienyl, wherein benzyl, phenyl, pyridyl, thienyl can in each case be unsubstituted or monosubstituted or polysubstituted with one or more substituents selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; C₃₋₁₀-cycloalkyl or heterocyclyl, in each case saturated or unsaturated, unsubstituted or monosubstituted or polysubstituted with one or more substituents selected independently of one another from the group consisting of F, Cl, Br, I, OH, ═O, C₁₋₄-alkyl, O—C₁₋₄-alkyl, OCF₃, C(═O)—OH and CF₃; or C₃₋₁₀-cycloalkyl or heterocyclyl bridged via C₁₋₈-alkyl, in each case saturated or unsaturated, unsubstituted or monosubstituted or polysubstituted with one or more substituents in each case selected independently of one another from the group consisting of F, Cl, Br, I, OH, ═O, C₁₋₄-alkyl, O—C₁₋₄-alkyl, OCF₃, C(═O)—OH and CF₃, wherein the alkyl chain can in each case be branched or unbranched, saturated or unsaturated, unsubstituted, monosubstituted or polysubstituted with one or more substituents in each case selected independently of one another from the group consisting of F, Cl, Br, I, OH, ═O and O—C₁₋₄-alkyl; aryl or heteroaryl, in each case unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₈-alkyl, SCF₃, S(═O)₂OH, benzyl, phenyl, pyridyl and thienyl, wherein benzyl, phenyl, pyridyl, thienyl can in each case be unsubstituted or monosubstituted or polysubstituted with one or more substituents selected independently from one another from the group consisting of F, Cl, Br, I, NO₂, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; or aryl or heteroaryl bridged via C₁₋₈-alkyl, in each case unsubstituted or monosubstituted or polysubstituted with one or more substituents in each case selected independently from one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₈-alkyl, SCF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents selected independently from one another from the group consisting of F, Cl, Br, I, NO₂, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH, wherein the alkyl chain can in each case be branched or unbranched, saturated or unsaturated, unsubstituted, monosubstituted or polysubstituted with one or more substituents selected in each case independently of one another from the group consisting of F, Cl, Br, I, OH, ═O and O—C₁₋₄-alkyl.

Preferably the radical
R² denotes H; F; Cl; Br; I; CN; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; C₁₋₁₀-alkyl, saturated or unsaturated, branched or unbranched, unsubstituted or monosubstituted or polysubstituted with one or more substituents in each case selected independently of one another from the group consisting of F, Cl, Br, I, CN, OH, ═O, O—C₁₋₄-alkyl, OCF₃, CF₃, NH₂, NH(C₁₋₄-alkyl), N(C₁₋₄-alkyl)₂, SH, S—C₁₋₄-alkyl, SCF₃; C₃₋₁₀-cycloalkyl, saturated or unsaturated, unsubstituted or monosubstituted or polysubstituted with one or more substituents selected independently of one another from the group consisting of F, Cl, Br, I, OH, ═O, C₁₋₄-alkyl, O—C₁₋₄-alkyl, OCF₃ and CF₃; or C₃₋₁₀-cycloalkyl bridged via C₁₋₈-alkyl, saturated or unsaturated, unsubstituted or monosubstituted or polysubstituted with one or more substituents selected independently of one another from the group consisting of F, Cl, Br, I, OH, ═O, C₁₋₄-alkyl, O—C₁₋₄-alkyl, OCF₃ and CF₃, wherein the alkyl chain can in each case be branched or unbranched, saturated or unsaturated or unsubstituted; aryl or heteroaryl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₈-alkyl, SCF₃, benzyl, phenyl, pyridyl and thienyl, wherein benzyl, phenyl, pyridyl, thienyl can in each case be unsubstituted or monosubstituted or polysubstituted with one or more substituents selected independently of one another from the group consisting of F, Cl, Br, I, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; or aryl or heteroaryl bridged via C₁₋₈-alkyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₈-alkyl, SCF₃, benzyl, phenyl, pyridyl and thienyl, wherein benzyl, phenyl, pyridyl, thienyl can in each case be unsubstituted or monosubstituted or polysubstituted with one or more substituents selected independently of one another from the group consisting of F, Cl, Br, I, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH, wherein the alkyl chain can in each case be branched or unbranched, saturated or unsaturated, and unsubstituted.

Particularly preferably
R² denotes H; F; Cl; Br; I; CN; C₁₋₁₀-alkyl, saturated or unsaturated, branched or unbranched, unsubstituted or monosubstituted or polysubstituted with one or more substituents selected independently of one another from the group consisting of F, Cl, Br, I and OH; C₃₋₁₀-cycloalkyl, saturated or unsaturated, unsubstituted; or C₃₋₁₀-cycloalkyl bridged via C₁₋₄-alkyl, saturated or unsaturated, unsubstituted, wherein the alkyl chain can be branched or unbranched, saturated or unsaturated, or unsubstituted; or phenyl, pyridyl, thienyl, in each case unsubstituted or monosubstituted or polysubstituted with one or more substituents selected independently of one another from the group consisting of C₁₋₄-alkyl, O—C₁₋₄-alkyl, F, Cl, Br, I, CF₃, OCF₃, OH, SH and SCF₃; or phenyl, pyridyl or thienyl bridged via C₁₋₄-alkyl, in each case unsubstituted or monosubstituted or polysubstituted with one or more substituents selected independently of one another from the group consisting of C₁₋₄-alkyl, O—C₁₋₄-alkyl, F, Cl, Br, I, CF₃, OCF₃, OH, SH and SCF₃, wherein the alkyl chain can be branched or unbranched, saturated or unsaturated, or unsubstituted.

Most particularly preferably the substituent
R² is selected from the group consisting of H; F; Cl; Br; I; CN; cyclopropyl; cyclobutyl; C₁₋₁₀-alkyl, saturated or unsaturated, branched or unbranched, unsubstituted or monosubstituted or polysubstituted with one or more substituents selected independently of one another from the group consisting of F, Cl, Br; phenyl, unsubstituted or monosubstituted or polysubstituted with one or more substituents selected independently of one another from the group consisting of C₁₋₄-alkyl, O—C₁₋₄-alkyl, F, Cl, Br, I, CF₃ and OCF₃.

Especially preferably the substituent
R² denotes H; F; Cl; Br; I; CF₃; CN; methyl; ethyl; n-propyl; iso-propyl; n-butyl; sec.-butyl; tert.-butyl; cyclopropyl; cyclobutyl; phenyl, unsubstituted or monosubstituted or polysubstituted with one or more substituents selected independently of one another from the group consisting of C₁₋₄-alkyl, O—C₁₋₄-alkyl, F, Cl, Br, I, CF₃ and OCF₃;

In a particularly preferred embodiment of the compounds according to the invention of the general formula (I) the radical R²≠H.

Especially preferably R² denotes tert.-butyl or CF₃.

In a further preferred embodiment of the compounds according to the invention of the general formula (I)
X denotes CR³ or N, preferably CR³,
   wherein R³ denotes H; C₁₋₁₀-alkyl, saturated or unsaturated, branched or unbranched, unsubstituted, monosubstituted or polysubstituted with one or more substituents in each case selected independently of one another from the group consisting of F, Cl, Br, I and OH;

Preferably
X denotes CR³ or N, preferably CR³,
   wherein R³ denotes H; C₁₋₁₀-alkyl, saturated or unsaturated, branched or unbranched, unsubstituted; or denotes CF₃.

Particularly preferably
X denotes CR³ or N, preferably CR³,
   wherein R³ denotes H; methyl; ethyl; n-propyl; iso-propyl; n-butyl; sec.-butyl; tert.-butyl; or CF₃.

Most particularly preferably
X denotes CR³ or N, preferably CR³,
   wherein R³ denotes H.

In a further preferred embodiment of the compounds according to the invention of the general formula (I)
p denotes 1 or 2, preferably 1.

In a further preferred embodiment of the compounds according to the invention of the general formula (I) the radical
R⁴ denotes H; C₁₋₁₀-alkyl, saturated or unsaturated, branched or unbranched, unsubstituted or monosubstituted or polysubstituted with one or more substituents in each case selected independently of one another from the group consisting of F, Cl, Br; I, OH and O—C₁₋₄-alkyl, preferably denotes H;

Preferably the radical
R⁴ denotes H; or C₁₋₁₀-alkyl, saturated or unsaturated, branched or unbranched, unsubstituted; more preferably denotes H.

Particularly preferably the radical
R⁴ denotes H; methyl; ethyl; n-propyl; or iso-propyl; preferably denotes H.

Most particularly preferably the radical
R⁴ denotes H.

In a further preferred embodiment of the compounds according to the invention of the general formula (I) the radicals
R⁵, R⁶ and R⁸ in each case denote independently of one another H; OH; C₁₋₁₀-alkyl, saturated or unsaturated, branched or unbranched, unsubstituted or monosubstituted or polysubstituted with one or more substituents in each case selected independently of one another from the group consisting of O—C₁₋₄-alkyl, F, Cl, Br, I and OH.

Preferably the radicals
R⁵, R⁶ and R⁸ in each case denote independently of one another H; C₁₋₁₀-alkyl, saturated or unsaturated, branched or unbranched, or unsubstituted.

Particularly preferably the radicals
R⁵, R⁶ and R⁸ denote in each case independently of one another H.

In a further preferred embodiment of the compounds according to the invention of the general formula (I) the radical
R^{7a} denotes the partial structure (T2)

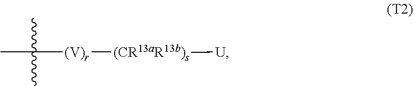

in which
V denotes C(═O), C(═O)NH, C(═O)—N(C₁₋₁₀-alkyl), or S(═O)₂,
   or
V denotes NH, N(C₁₋₁₀-alkyl) or NH—S(═O)₂, if T denotes CH,
r denotes 0 or 1, preferably 0;
R^{13a} and R^{13b} in each case denote independently of one another H; F; Cl; Br; I; NO₂; CF₃; CN; OH; OCF₃; NH₂; C₁₋₄-alkyl, O—C₁₋₄-alkyl, NH—C₁₋₄-alkyl, N(C₁₋₄-alkyl)₂, in each case saturated or unsaturated, branched or unbranched, unsubstituted or monosubstituted or polysubstituted with one or more substituents in each case selected independently of one another from the group consisting of F, Cl, Br, I, O—C₁₋₄-alkyl, OH and OCF₃;
   with the proviso that if R^{13a} and R^{13b} are bonded to the same carbon atom, only one of the substituents R^{13a} and R^{13b} can denote OH; OCF₃; NH₂; O—C₁₋₄-alkyl, NH—C₁₋₄-alkyl or N(C₁₋₄-alkyl)₂;
s denotes 0, 1, 2, 3 or 4;
U denotes C₁₋₄-alkyl, saturated or unsaturated, branched or unbranched, unsubstituted or monosubstituted or polysubstituted with one or more substituents in each case selected independently of one another from the group consisting of F, Cl, Br, I, NO₂, CN, OH, ═O, O—C₁₋₄-alkyl, OCF₃, C(═O)—OH, CF₃, NH₂, NH(C₁₋₄-alkyl), N(C₁₋₄-alkyl)₂, SH, S—C₁₋₄-alkyl, SCF₃ and S(═O)₂OH; C₃₋₁₀-cycloalkyl or heterocyclyl, in each case saturated or unsaturated, unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents selected independently of one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; aryl or heteroaryl, in each case unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, C₁₋₄-alkyl monosubstituted or disubstituted with OH, C(═O)—OH, CF₃, NH₂, NH(C₁₋₄-alkyl), N(C₁₋₄-alkyl)₂, SH, S—C₁₋₄-alkyl, SCF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents selected independently of one another from the group consisting of F, Cl, Br, I, NO₂, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH,
   or
U denotes H if at least one of the parameters r or s≠0.

If s≠0, then the radicals R^{13a} and R^{13b}, having regard to the aforementioned proviso, can on the same carbon atom as well as on different carbon atoms in each case denote independently of one another H; F; Cl; Br; I; NO₂; CF₃; CN; OH; OCF₃; NH₂; C₁₋₄-alkyl, O—C₁₋₄-alkyl, NH—C₁₋₄-alkyl, N(C₁₋₄-alkyl)₂, wherein C₁₋₄-alkyl can in each case be saturated or unsaturated, branched or unbranched, unsubstituted or monosubstituted or polysubstituted with one or more substituents in each case selected independently of one another from the group consisting of F, Cl, Br, I, O—C₁₋₄-alkyl, OH and OCF₃.

Preferably the radical R^{7a} denotes the partial structure (T2),

in which
V denotes C(═O) or S(═O)₂,
r denotes 0 or 1, preferably 0;
R^{13a} and R^{13b} in each case independently of one another denote H; F; Cl; Br; I; NO₂; CF₃; CN; methyl; ethyl; n-propyl; iso-propyl; n-butyl; sec.-butyl; tert.-butyl; CH₂CF₃; OH; O-methyl; O-ethyl; O—(CH₂)₂—O—CH₃; O—(CH₂)₂—OH; OCF₃; NH₂; NH-methyl; N(methyl)₂; NH-ethyl; N(ethyl)₂; or N(methyl)(ethyl);
   with the proviso that if R^{13a} and R^{13b} are bonded to the same carbon atom, only one of the substituents R^{11a} and R^{11b} can denote OH; OCF₃; O-methyl; O-ethyl; O—(CH₂)₂—O—CH₃; O—(CH₂)₂—OH; NH₂; NH-methyl; N(methyl)₂; NH-ethyl; N(ethyl)₂; or N(methyl)(ethyl);
s denotes 0, 1, 2, 3 or 4; preferably 0, 1 or 2;
U denotes C₁₋₄-alkyl, saturated or unsaturated, branched or unbranched, unsubstituted or monosubstituted or polysubstituted with one or more substituents in each case selected independently of one another from the group consisting of F, Cl, Br, I, NO₂, CN, OH, ═O, O—C₁₋₄-alkyl, OCF₃, CF₃, NH₂, NH(C₁₋₄-alkyl), N(C₁₋₄-alkyl)₂, SH, S—C₁₋₄-alkyl and SCF₃; C₃₋₁₀-cycloalkyl or heterocyclyl, in each case saturated or unsaturated, unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₄-alkyl, SCF₃, benzyl and phenyl, wherein benzyl and phenyl can in each case be unsubstituted or monosubstituted or polysubstituted with one or more substituents selected independently of one another from the group consisting of F, Cl, Br, I, NO₂, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₄-alkyl, SCF₃; and phenyl, pyridyl and thienyl, in each case unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, C₁₋₄-alkyl monosubstituted or disubstituted with OH, CF₃, NH₂, NH(C₁₋₄-alkyl), N(C₁₋₄-alkyl)₂, SH, S—C₁₋₄-alkyl, SCF₃, benzyl, phenyl, pyridyl and thienyl, wherein benzyl, phenyl, pyridyl and thienyl can in each case be unsubstituted or monosubstituted or polysubstituted with one or more substituents selected independently of one another from the group consisting of F, Cl, Br, I, NO₂, CN, OH, O—C₁₋₈-alkyl, OCF₃, C₁₋₄-alkyl, CF₃, NH₂, NH(C₁₋₄-alkyl), N(C₁₋₄-alkyl)₂, SH, S—C₁₋₄-alkyl and SCF₃;

### Or

U denotes H if at least one of the parameters r or s≠0.

If s≠0, then the radicals R^{13a} and R^{13b}, having regard to the aforementioned proviso, can on the same carbon atom as well as on different carbon atoms denote in each case independently of one another H; F; Cl; Br; I; NO₂; CF₃; CN; methyl; ethyl; n-propyl; iso-propyl; n-butyl; sec.-butyl; tert.-butyl; CH₂CF₃; OH; O-methyl; O-ethyl; O—(OH₂)₂—O—OH₃; O—(CH₂)₂—OH; OCF₃; NH₂; NH-methyl; N(methyl)₂; NH-ethyl; N(ethyl)₂; or N(methyl)(ethyl).

Particularly preferably the radical R^{7a} denotes the partial structure (T2),

in which
V denotes C(═O) or S(═O)₂,
r denotes 0 or 1, preferably 0;
R^{13a} and R^{13b} in each case denote independently of one another H; F; Cl; Br; I; methyl; ethyl; n-propyl; iso-propyl; n-butyl; sec.-butyl; tert.-butyl; OH; O-methyl; O-ethyl;
   with the proviso that if R^{13a} and R^{13b} are bonded to the same carbon atom, only one of the substituents R^{11a} and R^{11b} can denote OH; O-methyl; O-ethyl;
s denotes 0, 1, 2, 3 or 4; preferably 0, 1 or 2;
U denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-alkyl, OCF₃, and CF₃; C₃₋₁₀-cycloalkyl, saturated or unsaturated, morpholinyl, thiomorpholinyl, piperidinyl, pyrrolidinyl, 4-methylpiperazinyl, piperazinyl, in each case unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₄-alkyl, SCF₃; phenyl, pyridyl or thienyl, in each case unsubstituted or monosubstituted or polysubstituted with one or more substituents in each case selected independently of one another from the group consisting of F, Cl, Br, I, NO₂, CN, OH, O—C₁₋₄-alkyl, OCF₃, C₁₋₄-alkyl, or C₁₋₄-alkyl monosubstituted or disubstituted with OH, CF₃, NH₂, NH(C₁₋₄-alkyl), N(C₁₋₄-alkyl)₂, SH, S—C₁₋₄-alkyl and SCF₃,
   or
U denotes H if at least one of the parameters r or s≠0.

If s≠0, then the radicals R^{13a} and R^{13b}, having regard to the aforementioned proviso, can on the same carbon atom as well as on different carbon atoms denote in each case independently of one another H; F; Cl; Br; I; methyl; ethyl; n-propyl; iso-propyl; n-butyl; sec.-butyl; tert.-butyl; OH; O-methyl; O-ethyl.

Most particularly preferably the radical R^{7a} denotes the partial structure (T2),

in which
V denotes C(═O) or S(═O)₂,
r denotes 0 or 1, preferably 0;
R^{13a} and R^{13b} in each case denote independently of one another H; methyl; ethyl; n-propyl; iso-propyl; n-butyl; sec.-butyl; tert.-butyl;
s denotes 0, 1, 2, 3 or 4; preferably denotes 0, 1 or 2;
U denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-Alkyl, OCF₃, and CF₃; C₃₋₁₀-cycloalkyl, saturated or unsaturated, morpholinyl, piperidinyl, pyrrolidinyl, 4-methylpiperazinyl, piperazinyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃; phenyl, pyridyl or thienyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, C₁₋₄-alkyl monosubstituted or disubstituted with OH, CF₃;
   or
U denotes H if at least one of the parameters r or s≠0.

If s≠0, then the radicals R^{13a} and R^{13b}, having regard to the aforementioned proviso, can on the same carbon atom as well as on different carbon atoms denote in each case independently of one another H; methyl; ethyl; n-propyl; iso-propyl; n-butyl; sec.-butyl; tert.-butyl.

Particularly preferably R^{7a} denotes the partial structure (T2),

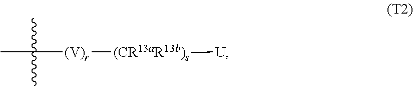

in which
V denotes C(═O) or S(═O)₂,
r denotes 0 or 1,
R^{13a} and R^{13b} in each case denote independently of one another H; methyl; ethyl; n-propyl; iso-propyl; n-butyl; sec.-butyl; tert.-butyl;
s denotes 0, 1, 2, 3 or 4; preferably 0, 1 or 2;
U denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-alkyl, OCF₃, and CF₃; C₃₋₁₀-cycloalkyl, saturated or unsaturated, morpholinyl, piperidinyl, pyrrolidinyl, 4-methylpiperazinyl, piperazinyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃; phenyl, pyridyl or thienyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃,
   or
U denotes H if at least one of the parameters r or s≠0.

If s≠0, then the radicals R^{13a} and R^{13b}, having regard to the aforementioned proviso, can on the same carbon atom as well as on different carbon atoms denote in each case independently of one another H; methyl; ethyl; n-propyl; iso-propyl; n-butyl; sec.-butyl; tert.-butyl.

In a further preferred embodiment the radical
R^{7b} denotes H; F; Cl; Br; or OH.

In another preferred embodiment the radical
R^{7b} denotes H; or OH.

In another preferred embodiment the radical
R^{7b} denotes H; F; or OH.

In another particularly preferred embodiment the radical
R^{7b} denotes H.

In a further, particularly preferred embodiment the compounds according to the invention of the general formula (I) have the general formula (Ie)

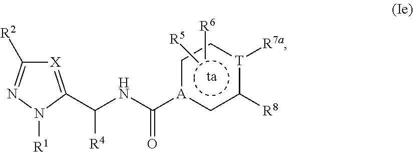

in which
X denotes CR³ or N, preferably CR³;
   wherein R³ denotes H; C₁₋₁₀-alkyl, saturated or unsaturated, branched or unbranched, unsubstituted;
A denotes N, C or CH;
T denotes N, C or CR^{7b},
   wherein R^{7b} denotes H; F; Cl; Br; I; or OH;
the symbol [symbol] denotes that the non-aromatic ring to can optionally contain at least one, preferably just one, unsaturated bond,
   with the proviso that if A denotes N, A is not part of an unsaturated bond, and
   with the proviso that if T denotes N, then T is not part of an unsaturated bond,
R¹ denotes the partial structure (T1-1)

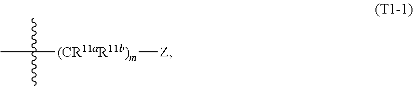

in which
   R^{11a} and R^{11b} in each case denote independently of one another H; methyl; ethyl; n-propyl; iso-propyl; n-butyl; sec.-butyl; tert.-butyl;
m denotes 0, 1 or 2;
Z denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-alkyl; C₃₋₁₀-cycloalkyl¹, saturated or unsaturated, morpholinyl, piperidinyl, 4-methylpiperazinyl, piperazinyl, in each case unsubstituted or monosubstituted or polysubstituted with one or more substituents in each case selected independently of one another from the group consisting of F, Cl, Br, I, OH, O—C₁₋₄-alkyl and C₁₋₄-alkyl; phenyl or pyridyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, C₁₋₄-alkyl monosubstituted or disubstituted with OH, CF₃, SH, S—C₁₋₄-alkyl, SCF₃;
R² denotes H; F; Cl; Br; I; CF₃; CN; methyl; ethyl; n-propyl; iso-propyl; n-butyl; sec.-butyl; tert.-butyl; cyclopropyl; cyclobutyl; phenyl, unsubstituted or monosubstituted or polysubstituted with one or more substituents selected independently of one another from the group consisting of C₁₋₄-alkyl, O—C₁₋₄-alkyl, F, Cl, Br, I, CF₃ and OCF₃;
R⁴ denotes H; methyl; ethyl; n-propyl; or iso-propyl; preferably denotes H;
R⁵, R⁶ and R⁸ denote in each case independently of one another H; C₁₋₁₀-alkyl, saturated or unsaturated, branched or unbranched, unsubstituted;
R^{7a} denotes the partial structure (T2)

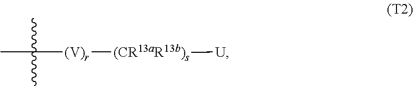

in which
V denotes C(═O) or S(═O)₂,
r denotes 0 or 1,
R^{13a} and R^{13b} in each denote independently of one another H; methyl; ethyl; n-propyl; iso-propyl; n-butyl; sec.-butyl; tert.-butyl;
s denotes 0, 1, 2, 3 or 4; preferably 0, 1 or 2;
U denotes C₁₋₄-alkyl, saturated or unsaturated, branched or unbranched, unsubstituted or monosubstituted or polysubstituted with one or more substituents in each case selected independently of one another from the group consisting of F, Cl, Br, I, OH, O—C₁₋₄-Alkyl, OCF₃, and CF₃; C₃₋₁₀-cycloalkyl, saturated or unsaturated, morpholinyl, piperidinyl, pyrrolidinyl, 4-methylpiperazinyl, piperazinyl, in each case unsubstituted or monosubstituted or polysubstituted with one or more substituents in each case selected independently of one another from the group consisting of F, Cl, Br, I, CN, OH, O—C₁₋₄-alkyl, OCF₃, C₁₋₄-alkyl, CF₃; phenyl, pyridyl or thienyl, in each case unsubstituted or monosubstituted or polysubstituted with one or more substituents in each case selected independently of one another from the group consisting of F, Cl, Br, I, CN, OH, O—C₁₋₄-Alkyl, OCF₃, C₁₋₄-Alkyl, CF₃,
or
U denotes H if at least one of the parameters r or s≠0.

Particularly preferred are compounds of the group
1 N-((3-tert-butyl-1-(3-chlorophenyl)-1H-pyrazol-5-yl)methyl)-4-(3-(trifluoromethyl)pyridin-2-yl)piperazine-1-carboxamide;
2 N-((3-tert-butyl-1-(3-chlorophenyl)-1H-pyrazol-5-yl)methyl)-4-(3-chloropyridin-2-yl)piperazine-1-carboxamide;
3 N-((1-(3-chlorophenyl)-3-(trifluoromethyl)-1H-pyrazol-5-yl)methyl)-4-(3-chloropyridin-2-yl)piperazine-1-carboxamide;
4 N-((1-(4-chlorophenyl)-3-(trifluoromethyl)-1H-pyrazol-5-yl)methyl)-4-(3-chloropyridin-2-yl)piperazine-1-carboxamide;
5 N-((3-tert-butyl-1-(3-chlorophenyl)-1H-pyrazol-5-yl)methyl)-4-(2-fluorophenyl)piperazine-1-carboxamide;
6 N-((3-tert-butyl-1-(3-chloro-4-fluorophenyl)-1H-pyrazol-5-yl)methyl)-4-(3-chloropyridin-2-yl)piperazine-1-carboxamide;
7 N-((3-tert-butyl-1-(pyridin-2-yl)-1H-pyrazol-5-yl)methyl)-4-(3-chloropyridin-2-yl)piperazine-1-carboxamide;
8 4-(3-chloropyridin-2-yl)-N-((1-m-tolyl-3-(trifluoromethyl)-1H-pyrazol-5-yl)methyl)piperazine-1-carboxamide;
9 N-((1-(3-chlorophenyl)-4-methyl-3-(trifluoromethyl)-1H-pyrazol-5-yl)methyl)-4-(3-chloropyridin-2-yl)piperazine-1-carboxamide;
10 N-((1-(3-chlorophenyl)-3-cyclopropyl-1H-pyrazol-5-yl)methyl)-4-(3-chloropyridin-2-yl)piperazine-1-carboxamide;
11 4-(3-chloropyridin-2-yl)-N-((1-(4-methoxybenzyl)-3-(trifluoromethyl)-1H-pyrazol-5-yl)methyl)piperazine-1-carboxamide;
12 4-(3-chloropyridin-2-yl)-N-((1-pentyl-3-(trifluoromethyl)-1H-pyrazol-5-yl)methyl)piperazine-1-carboxamide;
13 4-(3-chloropyridin-2-yl)-N-((1-(tetrahydro-2H-pyran-4-yl)-3-(trifluoromethyl)-1H-pyrazol-5-yl)methyl)piperazine-1-carboxamide;
14 N-((1-(3-chlorophenyl)-3-(trifluoromethyl)-1H-pyrazol-5-yl)methyl)-4-methylpiperazine-1-carboxamide;
15 N-((1-(3-chlorophenyl)-3-(trifluoromethyl)-1H-pyrazol-5-yl)methyl)-4-ethylpiperazine-1-carboxamide;
16 4-tert-butyl-N-((1-(3-chlorophenyl)-3-(trifluoromethyl)-1H-pyrazol-5-yl)methyl)piperazine-1-carboxamide;
17 N-((1-(3-chlorophenyl)-3-(trifluoromethyl)-1H-pyrazol-5-yl)methyl)-4-cyclohexylpiperazine-1-carboxamide;
18 N-((1-(3-chlorophenyl)-3-(trifluoromethyl)-1H-pyrazol-5-yl)methyl)-4-(thiophen-2-yl)piperazine-1-carboxamide;
19 N-((1-(3-chlorophenyl)-3-(trifluoromethyl)-1H-pyrazol-5-yl)methyl)-4-phenylpiperazine-1-carboxamide;
20 4-benzyl-N-((3-tert-butyl-1-(3-chlorophenyl)-1H-pyrazol-5-yl)methyl)piperazine-1-carboxamide;
21 N-((3-tert-butyl-1-(3-chlorophenyl)-1H-pyrazol-5-yl)methyl)-4-(1-phenylethyl)piperazine-1-carboxamide;
22 N-((1-(3-chlorophenyl)-3-(trifluoromethyl)-1H-pyrazol-5-yl)methyl)-4-(1-(4-fluorophenyl)ethyl)piperazine-1-carboxamide;
23 N-((1-(3-chlorophenyl)-3-(trifluoromethyl)-1H-pyrazol-5-yl)methyl)-4-(methylsulfonyl)piperazine-1-carboxamide;
24 4-acetyl-N-((1-(3-chlorophenyl)-3-(trifluoromethyl)-1H-pyrazol-5-yl)methyl)piperazine-1-carboxamide;
25 4-benzoyl-N-((1-(3-chlorophenyl)-3-(trifluoromethyl)-1H-pyrazol-5-yl)methyl)piperazine-1-carboxamide;
26 N-((1-(3-chlorophenyl)-3-(trifluoromethyl)-1H-pyrazol-5-yl)methyl)-4-phenylpiperidine-1-carboxamide;
27 N-((1-(3-chlorophenyl)-3-(trifluoromethyl)-1H-pyrazol-5-yl)methyl)-4-(2-methoxyphenyl)piperidine-1-carboxamide;
28 N-((1-(3-chlorophenyl)-3-(trifluoromethyl)-1H-pyrazol-5-yl)methyl)-4-(2,4-difluorophenyl)piperidine-1-carboxamide;
29 N-((1-(3-chlorophenyl)-3-(trifluoromethyl)-1H-pyrazol-5-yl)methyl)-4-hydroxy-4-phenylpiperidine-1-carboxamide;
30 N-((1-(3-chlorophenyl)-3-(trifluoromethyl)-1H-pyrazol-5-yl)methyl)-1-methylpiperidine-4-carboxamide;
31 1-acetyl-N-((1-(3-chlorophenyl)-3-(trifluoromethyl)-1H-pyrazol-5-yl)methyl)piperidine-4-carboxamide;
32 1-benzoyl-N-((1-(3-chlorophenyl)-3-(trifluoromethyl)-1H-pyrazol-5-yl)methyl)piperidine-4-carboxamide;
33 N-((1-(3-chlorophenyl)-3-(trifluoromethyl)-1H-pyrazol-5-yl)methyl)-4-isopropylcyclohexane carboxamide;
34 N-((1-(3-chlorophenyl)-3-(trifluoromethyl)-1H-pyrazol-5-yl)methyl)-4-(4-fluorophenyl)-5,6-dihydropyridine-1(2H)-carboxamide;
35 N-((1-(3-chlorophenyl)-3-(trifluoromethyl)-1H-pyrazol-5-yl)methyl)-4-hydroxycyclohex-1-ene carboxamide;
36 N-((1-(3-chlorophenyl)-3-(trifluoromethyl)-1H-pyrazol-5-yl)methyl)-1-ethyl-1,2,3,6-tetrahydropyridin-4-carboxamide;
37 N-((1-(3-chlorophenyl)-3-(trifluoromethyl)-1H-pyrazol-5-yl)methyl)-1-(4-fluorophenylsulfonyl)-1,2,3,6-tetrahydropyridine-4-carboxamide;
38 N-((1-(3-chlorophenyl)-3-(trifluoromethyl)-1H-pyrazol-5-yl)methyl)-4-ethylcyclohex-3-ene carboxamide;
39 (S)-4-(3-chlor-5-(1,2-dihydroxyethyl)pyridin-2-yl)-N-((1-(3-chlorophenyl)-3-(trifluoromethyl)-1H-pyrazol-5-yl)methyl)piperazine-1-carboxamide;
40 (S)-4-(3-chlor-5-(1,2-dihydroxyethyl)pyridin-2-yl)-N-((1-(3-chlorophenyl)-3-(trifluoromethyl)-1H-pyrazol-5-yl)methyl)-5,6-dihydropyridine-1(2H)-carboxamide;
41 (S)-4-(3-chlor-5-(1,2-dihydroxyethyl)pyridin-2-yl)-N-((1-(3-chlorophenyl)-3-(trifluoromethyl)-1H-pyrazol-5-yl)methyl)-4-fluoropiperidine-1-carboxamide;
42 N-((1-(3-chlorophenyl)-3-(trifluoromethyl)-1H-pyrazol-5-yl)methyl)-1-(3-chloropyridine-2-yl)-1,2,3,6-tetrahydropyridine-4-carboxamide;
43 N-((3-tert-butyl-1-(3-chlorophenyl)-1H-1,2,4-triazol-5-yl)methyl)-4-(3-chloropyridine-2-yl)piperazine-1-carboxamide;
44 N-((3-tert-butyl-1-(3-chlorophenyl)-1H-1,2,4-triazol-5-yl)methyl)-4-(1-(4-fluorophenyl)ethyl)piperazine-1-carboxamide;
45 4-(1-(4-fluorophenyl)ethyl)-N-((1-hexyl-3-(trifluoromethyl)-1H-1,2,4-triazol-5-yl)methyl)piperazine-1-carboxamide;

In each case in the form of the free compounds; the racemate; the enantiomers, diastereomers, mixtures of the enantiomers or diastereomers or an individual enantiomer or diasteromer; or in the form of the salts of physiologically compatible acids or bases; or in the form of solvates.

Particularly preferred are also compounds selected from the group consisting of the aforementioned compounds 1-45 and
46 N-((1-(3-chlorophenyl)-3-(trifluoromethyl)-1H-pyrazol-5-yl)methyl)-4-(2-fluorophenyl)piperazine-1-carboxamide;
47 N-((1-(3-chlorophenyl)-3-(trifluoromethyl)-1H-pyrazol-5-yl)methyl)-4-(4-fluorophenyl)piperazine-1-carboxamide;
48 N-((1-(3-chlorophenyl)-3-(trifluoromethyl)-1H-pyrazol-5-yl)methyl)-4-(2-methoxyphenyl)piperazine-1-carboxamide;
49 N-((1-(3-chlorophenyl)-3-(trifluoromethyl)-1H-pyrazol-5-yl)methyl)-4-(3-methoxyphenyl)piperazine-1-carboxamide;
50 N-((1-(3-chlorophenyl)-3-(trifluoromethyl)-1H-pyrazol-5-yl)methyl)-4-(4-methoxyphenyl)piperazine-1-carboxamide;
51 4-(2-chlorophenyl)-N-((1-(3-chlorophenyl)-3-(trifluoromethyl)-1H-pyrazol-5-yl)methyl)piperazine-1-carboxamide;
52 N-((3-tert-butyl-1-(3-chlorophenyl)-1H-pyrazol-5-yl)methyl)-4-(3-chlorophenyl)piperazine-1-carboxamide;
53 N-((3-tert-butyl-1-(3-chlorophenyl)-1H-pyrazol-5-yl)methyl)-4-(4-chlorophenyl)piperazine-1-carboxamide;
54 4-(4-chlorophenyl)-N-((1-(3-chlorophenyl)-3-(trifluoromethyl)-1H-pyrazol-5-yl)methyl)piperazine-1-carboxamide;
55 N-((3-tert-butyl-1-(3-chlorophenyl)-1H-pyrazol-5-yl)methyl)-4-(3-(trifluoromethyl)pyridin-2-yl)piperazine-1-carboxamide; and
56 N-((1-(3-chlorophenyl)-3-(trifluoromethyl)-1H-pyrazol-5-yl)methyl)-4-(3-(trifluoromethyl)pyridin-2-yl)piperazine-1-carboxamide;

In each case in the form of the free compounds; the racemate; the enantiomers, diastereomers, mixtures of the enantiomers or diastereomers or an individual enantiomer or diastereomer; or in the form of the salts of physiologically compatible acids or bases.

In addition compounds according to the invention of the general formula (I) may be preferred, that in the FLIPR assay with CHO K1 cells that have been transfixed with the human VR1 gene effect in a concentration of less than 2000 nM, preferably less than 1000 nM, particularly preferably less than 300 nM, most particularly preferably less than 100 nM, even more preferably less than 75 nM, still most preferably less than 50 nM, and most of all preferably less than 10 nM, a 50% displacement of capsaicin that is present in a concentration of 100 nM.

In this connection the Ca²⁺ inflow is quantified in the FLIPR assay with the aid of a Ca²⁺-sensitive dye (Type Fluo-4, Molecular Probes Europe BV, Leiden, Netherlands) in a fluorescent imaging plate reader (FLIPR, Molecular Devices, Sunnyvale, USA), as described hereinafter.

A further object of the present invention is a process for preparing compounds of the general formula (I) shown hereinbefore, according to which at least one compound of the general formula (II)

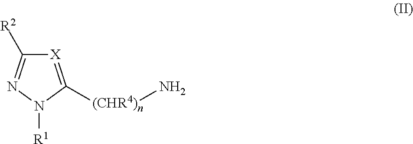

in which X, R¹, R², R⁴ and n have one of the aforementioned meanings, is reacted in a reaction medium, optionally in the presence of at least one suitable coupling reagent, optionally in the presence of at least one base, with a compound of the general formula (III) or (IV),

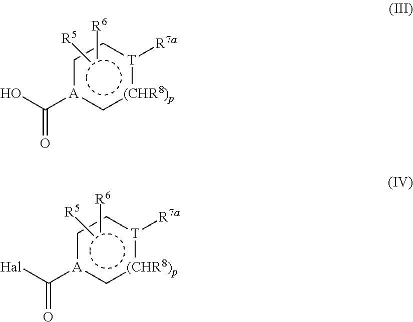

in which Hal denotes a halogen, preferably denotes Cl or Br, and R⁵, R⁶, R^{7a}, R⁸, p and T have one of the aforementioned meanings and A denotes CH or C, to form a compound of the general formula (I),

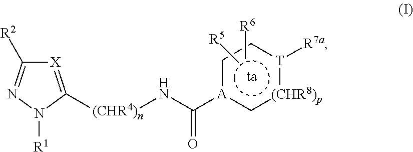

in which X, R¹, R², R⁴, R⁵, R⁶, R^{7a}, R⁸, n, p, [symbol] and T have one of the aforementioned meanings and A denotes CH or C;
or that at least one compound of the general formula (II),

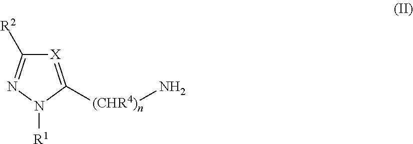

in which X, R¹, R², R⁴ and n have one of the aforementioned meanings is reacted in the presence of phenyl chloroformate, optionally in the presence of at least one base and/or coupling reagent, to form a compound of the general formula (V)

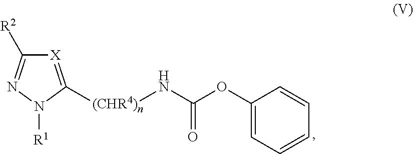

in which X, R¹, R², R⁴, and n have one of the aforementioned meanings, and this is optionally purified and/or isolated, and a compound of the general formula (V) is reacted with a compound of the general formula (VI),

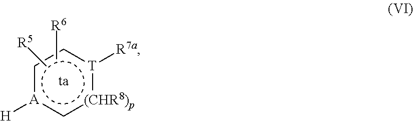

in which R⁵, R⁶, R^{7a}, R⁸, p and T have one of the aforementioned meanings and A denotes N, in a reaction medium, optionally in the presence of at least one suitable coupling reagent, optionally in the presence of at least one base, for form a compound of the general formula (I),

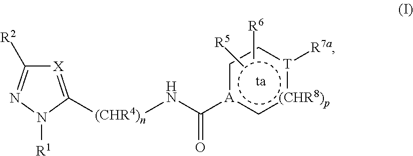

in which X R¹, R², R⁴, R⁵, R⁶, R^{7a}, R⁸, n, p, [symbol] and T have one of the aforementioned meanings and A denotes N.

The reaction of compounds of the general formulae (II) and (VI) shown hereinbefore with carboxylic acids of the general formula (III) shown hereinbefore to form compounds of the general formula (I) shown hereinbefore is preferably carried out in a reaction medium selected from the group consisting of diethyl ether, tetrahydrofuran, acetonitrile, methanol, ethanol, (1,2)-dichloroethane, dimethylformamide, dichloromethane and corresponding mixtures, optionally in the present of at least one coupling reagent, preferably selected from the group consisting of 1-benzotriazolyloxy-tris-(dimethylamino)-phosphonium hexafluorophosphate (BOP), dicyclohexylcarbodiimide (DCC), N′-(3-dimethylaminopropyl)-N-ethylcarbodiimide (EDCI), diisoproylcarbodiimide, 1,1′-carbonyl-diimidazole (CDI), N-(dimethyamino)-1H-1,2,3-triazolo[4,5-b]pyridino-1-ylmethylene]-N-methylmethanaminium hexafluorophosphate N-oxide (HATU), O-(benzotriazol-1-yl)-N,N,N′,N′-tetramethyluroniom hexafluorophosphate (HBTU), O-(benzotriazol-1-yl)-N,N,N′,N′-tetramethyluronium-tetrafluoroborate (TBTU), N-hydroxybenzotriazole (HOBt) and 1-hydroxy-7-azabenzotriazole (HOAt), optionally in the presence of at least one organic base, preferably selected from the group consisting of triethylamine, pyridine, dimethylaminopyridine, N-methylmorpholine and diisopropylethylamine, preferably at temperatures from −70° C. to 100° C.

Alternatively the reaction of compounds of the general formulae (II) and (VI) shown hereinbefore with carboxylic acid derivatives of the general formula (IV) shown hereinbefore, in which Hal denotes a halogen as leaving group, preferably a chlorine or bromine atom, to form compounds of the general formula (I) shown hereinbefore is carried out in a reaction medium preferably selected from the group consisting of diethyl ether, tetrahydrofuran, acetonitrile, methanol, ethanol, dimethylformamide, dichloromethane and corresponding mixtures, optionally in the presence of an organic or inorganic base, preferably selected from the group consisting of triethylamine, dimethylaminopyridine, pyridine and diisopropylamine, at temperatures from −70° C. to 100° C.

The compounds of the formulae (II), (III), (IV), (V) and (VI) shown hereinbefore are in each case commercially available and/or can be prepared by conventional processes known to the person skilled in the art.

The reactions described hereinbefore can be carried out in each case under the normal conditions known to the person skilled in the art, for example having regard to pressure or order of addition of the components. If necessary the optimal practical procedure under the respective conditions can be determined by the person skilled in the art by simple preliminary experiments. The intermediate products and end products obtained according to the reactions described hereinbefore can in each case, if desired and/or necessary, be purified and/or isolated by conventional methods known to the person skilled in the art. Suitable purification processes are for example extraction processes and chromatographic methods such as column chromatography or preparative chromatography. All the process steps described hereinbefore as well as in each case also the purification and/or isolation of intermediate products or end products can be carried out partly or completely under an inert gas atmosphere, preferably under a nitrogen atmosphere.

The substituted compounds according to the invention of the general formula (I) shown hereinbefore as well as corresponding stereoisomers can be isolated in the form of their free bases, their free acids and also in the form of corresponding salts, in particular physiologically compatible salts.

The free bases of the respective substituted compounds according to the invention of the general formula (I) shown hereinbefore as well as corresponding stereoisomers can be converted into the corresponding salts, preferably physiologically compatible salts, by for example reaction with an inorganic or organic acid, preferably with hydrochloric acid, hydrobromic acid sulfuric acid, methanesulfonic acid, p-toluenesulfonic acid, carbonic acid, formic acid, acetic acid, oxalic acid, succinic acid, tartaric acid, mandelic acid, fumaric acid, maleic acid, lactic acid, citric acid, glutamic acid, saccharic acid, monomethylsebacic acid, 5-oxo-proline, hexane-1-sulfonic acid, nicotinic acid, 2-, 3- or 4-aminobenzoic acid, 2,4,6-trimethylbenzoic acid, α-lipoic acid, acetylglycine, hippuric acid, phosphoric acid and/or aspartic acid. The free bases of the respective substituted compounds of the aforementioned general formula (I) and corresponding stereoisomers can likewise be converted with the free acid or a salt of a sugar substitute, such as e.g. saccharine, cyclamate or acesulfam, into the corresponding physiologically compatible salts Correspondingly, the free acids of the substituted compounds of the general formula (I) mentioned hereinbefore and corresponding stereoisomers can be converted into the corresponding physiologically compatible salts by reaction with a suitable base. Alkali metal salts, alkaline earth metal salts or ammonium salts [NHₓR^{4-x]}⁺, wherein x=0, 1, 2, 3 or 4 and R denotes a branched or unbranched C₁₋₄-alkyl radical, may be mentioned by way of example.

The substituted compounds according to the invention of the general formula (I) mentioned hereinbefore and corresponding stereoisomers can, like the corresponding acids, the corresponding bases or salts of these compounds, optionally also be obtained in the form of their solvates, preferably in the form of their hydrates, by conventional methods known to the person skilled in the art.

If the substituted compounds according to the invention of the general formula (I) mentioned hereinbefore are obtained after their preparation in the form of a mixture of their stereoisomers, preferably in the form of their racemates or other mixtures of their various enantiomers and/or diastereomers, then these can be separated and if necessary isolated by conventional methods known to the person skilled in the art. Chromatographic separation methods, in particular liquid chromatography methods under normal pressure or under elevated pressure, preferably MPLC and HPLC methods, as well as methods involving fractional crystallisation, may be mentioned by way of example. In this way, individual enantiomers and formed diastereomer salts in particular can be separated from one another, for example by means of HPLC on the chiral stationary phase or by means of crystallisation with chiral acids, for example (+)-tartaric acid, (−)-tartaric acid or (+)-10-camphorsulfonic acid.

The substituted compounds according to the invention of the general formula (I) mentioned hereinbefore and corresponding stereoisomers as well as in each case the corresponding acids, bases, salts and solvates are toxicologically harmless and are therefore suitable as pharmaceutical active substances in medicaments.

A further object of the present invention is accordingly a medicament containing at least one compound according to the invention of the general formula (I) shown hereinbefore, in each case optionally in the form of one of its pure stereoisomers, in particular enantiomers or diastereomers, its racemates or in the form of a mixture of stereoisomers, in particular of the enantiomers and/or diastereomers, in an arbitrary mixture ratio, or in each case in the form of a corresponding salt, or in each case in the form of a corresponding solvate, as well as optionally one or more pharmaceutically compatible auxiliary substances.

These medicaments according to the invention are suitable in particular for the vanilloid receptor 1 (VR1/TRPV1) regulation, preferably for vanilloid receptor 1 (VR1/TRPV1) inhibition and/or for vanilloid receptor 1 (VR1/TRPV1) stimulation, i.e. they have an agonistic or antagonistic action.

Likewise, the medicaments according to the invention are suitable for the prevention and/or treatment of disorders or diseases that are mediated at least in part by vanilloid receptors 1.

The medicament according to the invention is suitable for administration to adults and children, including infants and babies.

The medicament according to the invention can be present as a liquid, semi-solid or solid medicament form, for example in the form of injection solutions, drops, juices, syrups, sprays, suspensions, tablets, patches, capsules, plasters, suppositories, ointments, creams, lotions, gels, emulsions, aerosols or in multiparticulate form, for example in the form of pellets or granules, optionally pressed into tablet form, packed in capsules or suspended in a liquid, and can also be administered as such.

Apart from at least one substituted compound of the general formula (I) shown hereinbefore, optionally in the form of one of its pure stereoisomers, in particular enantiomers or diastereomers, its racemate or in the form of mixtures of the stereoisomers, in particular of the enantiomers or diastereomers, in an arbitrary mixture ratio, or optionally in the form of a corresponding salt or in each case in the form of a corresponding solvate, the medicament according to the invention usually contains further physiologically compatible pharmaceutical or auxiliary substances, which can be selected for example from the group consisting of carriers, fillers, solvents, diluents, surfactants, dyes, pigments, preservatives, disintegrants, lubricants, greases, aroma substances and binders.

The choice of the physiologically compatible auxiliary substances as well as the amounts thereof to be used depends on whether the medicament is to be administered orally, subcutaneously, parenterally, intravenously, intraperitoneally, intradermally, intramuscularly, intranasally, orally, rectally or topically, for example to treat infections of the skin, mucus membranes and eyes. For oral application preparations in the form of tablets, sugar-coated pills, capsules, granules, pellets, drops, juices and syrups are preferably suitable, while for parenteral, topical and inhalated application, solutions, suspensions, readily reconstitutable dry preparations as well as sprays are preferably suitable. The substituted compounds according to the invention to be used in the medicament according to the invention in a depot form, in dissolved form or in a plaster, optionally with the addition of agents promoting penetration of the skin, are suitable percutaneous application preparations. Orally or percutaneously usable preparation forms can also effect the delayed release of the respective substituted compound according to the invention.

The production of the medicaments according to the invention is carried out with the aid of conventional means, devices, equipment, methods and processes known from the prior art, as are described for example in “Remington's Pharmaceutical Sciences”, edited by A. R. Gennaro, 17th edition, Mack Publishing Company, Easton, Pa., 1985, in particular in Part 8, Chapters 76 to 93. The corresponding description is hereby introduced by way of reference and counts as part of the disclosure. The amount of the respective substituted compounds according to the invention of the general formula (I) shown hereinbefore to be administered to the patient may vary and depends for example on the weight or age of the patient as well as the method of application, medical indications and the severity of the disease. Normally 0.001 to 100 mg/kg, preferably 0.05 to 75 mg/kg, particularly preferably 0.05 to 50 mg/kg body weight of the patient of at least one such compound according to the invention are administered.

Preferably the medicament according to the invention is suitable for the treatment and/or prevention of one or more conditions and diseases selected from the group consisting of pain, preferably pain selected from the group consisting of acute pain, chronic pain, neuropathic pain and visceral pain; arthritic pain; hyperalgesia; allodynia; causalgia, migraine; depression; neuropathy; nerve injuries; neurodegenerative diseases, preferably selected from the group consisting of multiple sclerosis, Alzheimer's disease, Parkinson's disease and Huntington's chorea; cognitive dysfunctions, preferably cognitive deficiency states, particularly preferably memory disorders; epilepsy; respiratory pathway diseases, preferably selected from the group consisting of asthma, bronchitis and inflammation of the lungs (pneumonia); coughing; urinary incontinence; overactive bladder (OAB); diseases and/or injuries of the gastrointestinal tract; duodenal ulcers; gastric ulcers; irritable bowel syndrome; strokes; occular irritation; skin irritation; neurotic skin diseases; allergic skin diseases; psoriasis; leukodermia; Herpes simplex; inflammations, preferably inflammations of the stomach, eyes, bladder, skin or nasal mucosa; diarrhoea; pruritus; osteoporosis; arthritis; osteoarthritis; rheumatic diseases; eating disorders, preferably selected from the group consisting of bulimia, cachexia, anorexia and obesity; medicament dependence; medicament misuse; withdrawal symptoms in medicament misuse; development of tolerance to medicaments, preferably to natural or synthetic opioids; drug dependence; drug misuse; withdrawal symptoms in drug dependence; alcohol dependence; alcohol misuse and withdrawal symptoms in alcohol dependence; diuresis; antinatriuresis; to influence the cardiovascular system; to improve vigilance; to treat wounds and/or burns; to treat severed nerves; to increase libido; to modulate movement activity; for anxiolysis; for local anaesthesia and/or to inhibit undesired side effects, preferably selected from the group consisting of hyperthermia, hypertension and bronchial constriction triggered by the administration of vanilloid receptor 1 (VR1/TRPV1 receptors) agonists, preferably selected from the group consisting of capsaicin, resiniferatoxin, olvanil, arvanil, nuvanil and capsavanil.

Particularly preferably the medicament according to the invention is suitable for the treatment and/or prevention of one or more diseases selected from the group consisting of pain, preferably pain selected from the group consisting of acute pain, chronic pain, neuropathic pain and visceral pain; arthritic pain; migraine; depression; neurodegenerative diseases, preferably selected from the group consisting of multiple sclerosis, Alzheimer's disease, Parkinson's disease and Huntington's chorea; cognitive dysfunctions, preferably cognitive deficiency states, particularly preferably memory disorders; inflammations, preferably inflammations of the stomach, eyes, bladder, skin or nasal mucosa; urinary incontinence; overactive bladder (OAB); medicament dependence; medicament misuse; withdrawal symptoms in medicament dependence; development of tolerance to medicaments, preferably development of tolerance to natural or synthetic opioids; drug dependence; drug misuse; withdrawal symptoms in drug dependence; alcohol dependence; alcohol misuse and withdrawal symptoms in alcohol dependence.

Most particularly preferably the medicament according to the invention is suitable for the treatment and/or prevention of pain, preferably pain selected from the group consisting of acute pain, chronic pain, neuropathic pain and visceral pain, and/or urinary incontinence.

A further object of the present invention is the use of at least one compound according to the invention as well as optionally one or more pharmaceutically compatible auxiliary substances for the production of a medicament for vanilloid receptor 1 (VR1/TRPV1) regulation, preferably for vanilloid receptor 1 (VR1/TRPV1) inhibition and/or for vanilloid receptor 1 (VR1/TRPV1 stimulation.

Preferred is the use of at least one substituted compound according to the invention as well as optionally one or more pharmaceutically compatible auxiliary substances for the production of a medicament for the prevent and/or treatment of disorders or diseases that are mediated at least in part by vanilloid receptors 1.

Particularly preferred is the use of at least one compound according to the invention as well as optionally one or more pharmaceutically compatible auxiliary substances for the production of a medicament for the treatment and/or prevention of one or more conditions or diseases selected from the group consisting of pain, preferably pain selected from the group consisting of acute pain, chronic pain, neuropathic pain, visceral pain and arthritic pain.

Particularly preferred is the use of at least one compound according to the invention as well as optionally one or more pharmaceutically compatible auxiliary substances for the production of a medicament for the treatment and/or prevention of one or more conditions and diseases selected from the group consisting of hyperalgesia; allodynia; causalgia; migraine; depression; neuropathy; nerve injuries; neurodegenerative diseases, preferably selected from the group consisting of multiple sclerosis, Alzheimer's disease, Parkinson's disease and Huntington's chorea; cognitive dysfunctions, preferably cognitive deficiency states, particularly preferably memory disorders; epilepsy; respiratory pathway diseases, preferably selected from the group consisting of asthma, bronchitis and inflammation of the lungs (pneumonia); coughing; urinary incontinence; overactive bladder (OAB); diseases and/or injuries of the gastrointestinal tract; duodenal ulcers; gastric ulcers; irritable bowel syndrome; strokes; occular irritation; skin irritation; neurotic skin diseases; allergic skin diseases; psoriasis; leukodermia; Herpes simplex; inflammations, preferably inflammations of the stomach, eyes, bladder, skin or nasal mucosa; diarrhoea; pruritus; osteoporosis; arthritis; osteoarthritis; rheumatic diseases; eating disorders, preferably selected from the group consisting of bulimia, cachexia, anorexia and obesity; medicament dependence; medicament misuse; withdrawal symptoms in medicament misuse; development of tolerance to medicaments, preferably to natural or synthetic opioids; drug dependence; drug misuse; withdrawal symptoms in drug dependence; alcohol dependence; alcohol misuse and withdrawal symptoms in alcohol dependence; diuresis; antinatriuresis; to influence the cardiovascular system; to improve vigilance; to treat wounds and/or burns; to treat severed nerves; to increase libido; to modulate movement activity; for anxiolysis; for local anaesthesia and/or to inhibit undesired side effects, preferably selected from the group consisting of hyperthermia, hypertension and bronchial constriction triggered by the administration of vanilloid receptor 1 (VR1/TRPV1 receptors) agonists, preferably selected from the group consisting of capsaicin, resiniferatoxin, olvanil, arvanil, SDZ-249665, SDZ-249482, nuvanil and capsavanil.

Most particularly preferred is the use of at least one substituted compound according to the invention as well as optionally one or more pharmaceutically compatible auxiliary substances for the production of a medicament for the treatment and/or prevention of one or more diseases selected from the group consisting of pain, preferably pain selected from the group consisting of acute pain, chronic pain, neuropathic pain and visceral pain; arthritic pain; migraine; depression; neurodegenerative diseases, preferably selected from the group consisting of multiple sclerosis; Alzheimer's disease, Parkinson's disease and Huntington's chorea; cognitive dysfunctions, preferably cognitive deficiency states, particularly preferably memory disorders; inflammations, preferably inflammations of the stomach, eyes, bladder skin or nasal mucosa; urinary incontinence; an overactive bladder (OAB); medicament dependence; medicament misuse; withdrawal symptoms in medicament misuse; development of tolerance to medicaments, preferably to natural or synthetic opioids; drug dependence; drug misuse; and withdrawal symptoms in alcohol dependence.

Especially preferred is the use of at least one substituted compound according to the invention as well as optionally one or more pharmaceutically compatible auxiliary substances for the production of a medicament for the treatment and/or prevention of pain, preferably selected from the group consisting of acute pain, chronic pain, neuropathic pain and visceral pain, and/or urinary incontinence.

A further object of the present invention is at least one substituted compound according to the invention as well as optionally one or more pharmaceutically compatible auxiliary substances for use in vanilloid receptor 1 (VR1/TRPV1) regulation, preferably for vanilloid receptor 1 (VR1/TRPV1) inhibition and/or for vanilloid receptor 1 (VR1/TRPV1) stimulation.

Preferred is at least one substituted compound according to the invention as well as optionally one or more pharmaceutically compatible auxiliary substances for use in the prevention and/or treatment of disorders or diseases that are mediated at least partly by vanilloid receptors 1.

Particularly preferred is at least one compound according to the invention as well as optionally one or more pharmaceutically compatible auxiliary substances for use in the treatment and/or prevention of one or more conditions or diseases selected from the group consisting of pain, preferably pain selected from the group consisting of acute pain, chronic pain, neuropathic pain and visceral pain, and arthritic pain.

Particularly preferred is at least one compound according to the invention as well as optionally one or more pharmaceutically compatible auxiliary substances for use in the treatment and/or prevention of one or more conditions or diseases selected from the group consisting of hyperalgesia; allodynia; causalgia; migraine; depression; neuropathy; nerve injuries; neurodegenerative diseases, preferably selected from the group consisting of multiple sclerosis, Alzheimer's disease, Parkinson's disease and Huntington's chorea; cognitive dysfunctions, preferably cognitive deficiency states, particularly preferably memory disorders; epilepsy; respiratory pathway diseases, preferably selected from the group consisting of asthma, bronchitis and inflammation of the lungs (pneumonia); coughing; urinary incontinence; overactive bladder (OAB); diseases and/or injuries of the gastrointestinal tract; duodenal ulcers; gastric ulcers; irritable bowel syndrome; strokes; occular irritation; skin irritation; neurotic skin diseases; allergic skin diseases; psoriasis; leukodermia; Herpes simplex; inflammations, preferably inflammations of the stomach, eyes, bladder, skin or nasal mucosa; diarrhoea; pruritus; osteoporosis; arthritis; osteoarthritis; rheumatic diseases; eating disorders, preferably selected from the group consisting of bulimia, cachexia, anorexia and obesity; medicament dependence; medicament misuse; withdrawal symptoms in medicament misuse; development of tolerance to medicaments, preferably to natural or synthetic opioids; drug dependence; drug misuse; withdrawal symptoms in drug dependence; alcohol dependence; alcohol misuse and withdrawal symptoms in alcohol dependence; diuresis; antinatriuresis; to influence the cardiovascular system; to improve vigilance; to treat wounds and/or burns; to treat severed nerves; to increase libido; to modulate movement activity; for anxiolysis; for local anaesthesia and/or to inhibit undesired side effects, preferably selected from the group hyperthermia, hypertension and bronchial constriction triggered by the administration of vanilloid receptor 1 (VR1/TRPV1 receptors) agonists, preferably selected from the group consisting of capsaicin, resiniferatoxin, olvanil, arvanil, nuvanil and capsavanil.

Most particularly preferred is at least one compound according to the invention as well as optionally one or more pharmaceutically compatible auxiliary substances for use in the treatment and/or prevention of one or more conditions and diseases selected from the group consisting of pain, preferably pain selected from the group consisting of acute pain, chronic pain, neuropathic pain and visceral pain, and arthritic pain; migraine; depression; neurodegenerative diseases, preferably selected from the group consisting of multiple sclerosis, Alzheimer's disease, Parkinson's disease and Huntington's chorea; cognitive dysfunctions, preferably cognitive deficiency states, particularly preferably memory disorders; inflammations, preferably inflammations of the stomach, eyes, bladder skin or nasal mucosa; urinary incontinence; an overactive bladder (OAB); medicament dependence; medicament misuse; withdrawal symptoms in medicament misuse; development of tolerance to medicaments, preferably to natural or synthetic opioids; drug dependence; drug misuse; and withdrawal symptoms in alcohol dependence.

Especially preferred is at least one compound according to the invention as well as optionally one or more pharmaceutically compatible auxiliary substances for use in the treatment and/or prevention of pain, preferably selected from the group consisting of acute pain, chronic pain, neuropathic pain, visceral pain, and/or urinary incontinence.

A further object of the present invention is a use of at least one substituted compound according to the invention as well as optionally one or more pharmaceutically compatible auxiliary substances for vanilloid receptor 1 (VR1/TRPV1) regulation, preferably for vanilloid receptor 1 (VR1/TRPV1 inhibition and/or for vanilloid receptor 1 (VR1/TRPV1) stimulation, preferably characterised in that at least one substituted compound according to the invention is administered in a therapeutically effective amount to a human or mammal.

Preferred is the use of at least one substituted compound according to the invention as well as optionally one or more pharmaceutically compatible auxiliary substances for the prevention and/or treatment of disorders or diseases that a mediated at least partly by vanilloid receptors 1, preferably characterised in that a substituted compound according to the invention is administered in a therapeutically effective amount to a human or mammal.

Particularly preferred is the use of at least one compound according to the invention as well as optionally one or more pharmaceutically compatible auxiliary substances for the treatment and/or prevention of one or more conditions or diseases selected from the group consisting of pain, preferably pain selected from the group consisting of acute pain, chronic pain, neuropathic pain, visceral pain and arthritic pain, preferably characterised in that a substituted compound according to the invention is administered in a therapeutically effective amount to a human or mammal.

Particularly preferred is the use of at least one compound according to the invention as well as optionally one or more pharmaceutically compatible auxiliary substances for the treatment and/or prevention of one or more conditions or diseases selected from the group consisting of hyperalgesia; allodynia; causalgia; migraine; depression; neuropathy; nerve injuries; neurodegenerative diseases, preferably selected from the group consisting of multiple sclerosis, Alzheimer's disease, Parkinson's disease and Huntington's chorea; cognitive dysfunctions, preferably cognitive deficiency states, particularly preferably memory disorders; epilepsy; respiratory pathway diseases, preferably selected from the group consisting of asthma, bronchitis and inflammation of the lungs (pneumonia); coughing; urinary incontinence; overactive bladder (OAB); diseases and/or injuries of the gastrointestinal tract; duodenal ulcers; gastric ulcers; irritable bowel syndrome; strokes; occular irritation; skin irritation; neurotic skin diseases; allergic skin diseases; psoriasis; leukodermia; Herpes simplex; inflammations, preferably inflammations of the stomach, eyes, bladder, skin or nasal mucosa; diarrhoea; pruritus; osteoporosis; arthritis; osteoarthritis; rheumatic diseases; eating disorders, preferably selected from the group consisting of bulimia, cachexia, anorexia and obesity; medicament dependence; medicament misuse; withdrawal symptoms in medicament dependence; development of tolerance to medicaments, preferably to natural or synthetic opioids; drug dependence; drug misuse; withdrawal symptoms in drug dependence; alcohol dependence; alcohol misuse and withdrawal symptoms in alcohol dependence; for diuresis; for antinatriuresis; to influence the cardiovascular system; to improve vigilance; to treat wounds and/or burns; to treat severed nerves; to increase libido; to modulate movement activity; for anxiolysis; for local anaesthesia and/or to inhibit undesired side effects, preferably selected from the group consisting of hyperthermia, hypertension and bronchial constriction triggered by the administration of vanilloid receptor 1 (VR1/TRPV1 receptors) agonists, preferably selected from the group consisting of capsaicin, resiniferatoxin, olvanil, arvanil, SDZ-249665, SDZ-249482, nuvanil and capsavanil, preferably characterised in that at least one substituted compound according to the invention is administered in a therapeutically effective amount to a human or mammal.

Most particularly preferred is the use of at least one compound according to the invention as well as optionally one or more pharmaceutically compatible auxiliary substances for the treatment and/or prevention of one or more conditions and diseases selected from the group consisting of pain, preferably pain selected from the group consisting of acute pain, chronic pain, neuropathic pain and visceral pain; arthritic pain; migraine; depression; neurodegenerative diseases, preferably selected from the group consisting of multiple sclerosis; Alzheimer's disease, Parkinson's disease and Huntington's chorea; cognitive dysfunctions, preferably cognitive deficiency states, particularly preferably memory disorders; inflammations, preferably inflammations of the stomach, eyes, bladder skin or nasal mucosa; urinary incontinence; an overactive bladder (OAB); medicament dependence; medicament misuse; withdrawal symptoms in medicament misuse; development of tolerance to medicaments, preferably development of tolerance to natural or synthetic opioids; drug dependence; drug misuse; withdrawal symptoms in drug dependence; alcohol dependence; alcohol misuse and withdrawal symptoms in drug dependence, preferably characterised in that at least one substituted compound according to the invention is administered in a therapeutically effective amount to a human or mammal.

Especially preferred is the use of at least one compound according to the invention as well as optionally one or more pharmaceutically compatible auxiliary substances for the treatment and/or prevention of pain, preferably selected from the group consisting of acute pain, chronic pain, neuropathic pain and visceral pain, and/or urinary incontinence, preferably characterised in that at least one substituted compound according to the invention is administered in a therapeutically effective amount to a human or mammal.

### Pharmacological Methods

### I. Functional Investigation on the Vanilloid Receptor 1 (VR1/TRPV1 Receptor)

The agonistic and antagonistic action of the substances to be investigated on the vanilloid receptor 1 (VR1/TRPV1) of rats can be determined by the following assay. According to this assay the Ca²⁺ inflow through the receptor channel is quantified with the aid of Ca²⁺-sensitive dye (Type Fluo-4, Molecular Probes Europe BV, Leiden, Netherlands) in a Fluorescent Imaging Plate Reader (FLIPR, Molecular Devices, Sunnyvale, USA).

### Method:

Complete Medium: 50 mL HAMS F12 nutrient mixture (Gibco Invitrogen GmbH, Karlsruhe, Germany) with
10 Vol % FCS (foetal calf serum, Gibco Invitrogen GmbH, Karlsruhe, Germany, heat-inactivated);

### 2 mM L-glutamine (Sigma, Munich, Germany);

1 wt. % AA solution (antibiotic/antimycotic solution, PAA, Pasching, Austria)
and 25 ng/ml medium NGF (2.5 S, Gibco Invitrogen GmbH, Karlsruhe, Germany)
Cell Culture Plate: poly-D-lysine-coated, black 96-well plates with clear floor (96 well black/clear plate, BD Biosciences, Heidelberg, Germany) are additionally coated with laminine (Gibco Invitrogen GmbH, Karlsruhe, Germany) by diluting laminine to a concentration of 100 μg/mL with PBS (Ca—Mg-free PBS, Gibco Invitrogen GmbH, Karlsruhe, Germany). Aliquots are taken in a concentration of 100 μg/mL laminine and stored at −20° C. The aliquots are diluted with PBS in a ratio of 1:10 to 10 μg/mL laminine and 50 μL of the solution are pipetted each time into a well of the cell culture plate. The cell culture plates are incubated for at least 2 hours at 37° C., the supernatant solution is suctioned off, and the wells are washed twice in each case with PBS. The coated cell culture plates are preserved with supernatant PBS, which is removed immediately before charging the plates.

### Preparation of the Cells:

The spinal column is removed from decapitated rats and is placed directly in cold, i.e. kept in an ice bath, HBSS buffer (Hank's buffered saline solution, Gibco Invitrogen GmbH, Karlsruhe, Germany), to which 1 Vol % (volume percent) of AA solution (antibiotic/antimycotic solution, PAA, Pasching, Austria) has been added. The spinal column is separated longitudinally and removed together with fasciae from the vertebral canal. The dorsal root ganglia (DRGs) are then removed and preserved in turn in cold HBSS buffer to which 1 Vol % of an AA solution has been added. The DRGs completely freed from blood residues and spinal nerves are transferred in each case to 500 μL cold collagenase type 2 (PAA, Pasching, Austria) and incubated for 35 minutes at 37° C. After adding about 2.5 Vol-% of trypsin (PAA, Pasching, Austria) the DRGs are incubated for a further 10 minutes at 37° C. After the complete incubation the enzyme solution is carefully pipetted off and 500 μL complete medium are added to the DRGs left behind. The DRGs are in each case suspended several times, drawn up by means of a syringe through no. 1, no. 12 and no. 16 canullas and transferred to 50 mL Falcon test tubes, which are made up to 15 mL with complete medium. The contents of each Falcon test tube are filtered in each case through a 70 μm Falcon filter insert and centrifuged for 10 minutes at 1200 revolutions and RT. The resulting pellet is in each case taken up in 250 μL complete medium and the cell count is measured.

The number of cells in the suspension is adjusted to 3×10⁵ per mL, and 150 μL of this suspension is added in each case to a well of the cell culture plates coated as described above. The plates are allowed to stand for two to three days at 37° C., 5 Vol % CO₂ and 95% relative atmospheric humidity in the incubator. The cells are then charged with 2 μM Fluo-4 and 0.01 Vol % Pluronic F127 (Molecular Probes Europe BV, Leiden Netherlands) in HBSS buffer (Hank's buffered saline solution, Gibco Invitrogen GmbH, Karlsruhe, Germany) for 30 min at 37° C., washed 3 times with HBSS buffer and, after a further incubation for 15 minutes at RT, used in the FLIPR assay to measure the Ca²⁺. The Ca²⁺-dependent fluorescence is in this connection measured before and after addition of substances (λex=488 nm, λem=540 nm). The quantification is carried out by measuring the highest fluorescence intensity (FC, Fluorescence Counts) over time.

### FLIPR Assay:

The FLIPR protocol consists of 2 additions of substances. First of all the compounds to be tested (10 μM) are pipetted onto the cells and the Ca²⁺ inflow is compared with the control (capsaicin 10 μM). From this is obtained the figure in % activation referred to the Ca²⁺ signal after addition of 10 μM capsaicin (CP). After 5 minutes' incubation 100 nM capsaicin are added and the inflow of Ca²⁺ is again determined.

Desensitising agonists and antagonists lead to a suppression of the Ca²⁺ inflow. The % inhibition compared to the maximum achievable inhibition with 10 μM capsaicin is calculated. Triple determinations (n=3) are carried out and these are repeated in at least 3 independent experiments (N=4).

Starting from the percentage displacement caused by different concentrations of the compounds of the general formula I to be tested, IC₅₀ inhibiting concentrations that produce a 50% displacement of capsaicin were calculated. K, values for the test substances were obtained by reconversion using the (Cheng, Prusoff; Biochem. Pharmacol. 22, 3099-3108, 1973) relationship.

### II. Functional Investigations on the Vanilloid Receptor (VR1)

The agonistic and antagonistic action of the substances to be investigated on the vanilloid receptor (VR1) can also be determined with the following assay. According to this assay the Ca²⁺ inflow through the channel is quantified in the Fluorescent Imaging Plate Reader (FLIPR, Molecular Devices, Sunnyvale, USA) with the aid of a Ca²⁺ sensitive dye (Type Fluo-4, Molecular Probes, Europe BV, Leiden, Netherlands).

### Method:

Chinese hamster ovary cells (CHO K1 cells, European Collection of Cell Cultures (ECACC) Great Britain) are stably transfixed with the VR1 gene. For the functional investigations these cells are plated out on poly-D-lysine-coated, black 96-well plates with a clear floor (BD Biosciences, Heidelberg, Germany) in a density of 25,000 cells/well. The cells are incubated overnight at 37° C. and 5% CO₂ in a culture medium (Nutrient Mixture ‘am's F12, 10 Vol % FCS (fetal calf serum), 18 μg/ml L-proline. The following day the cells are incubated with Fluo-4 (Fluo-4 2 μM, Pluronic F127 0.01 Vol %, Molecular Probes in HBSS (Hank's buffered saline solution), Gibco Invitrogen GmbH, Karlsruhe, Germany) for 30 minutes at 37° C. The plates are then washed 3 times with HBSS buffer and, after a further incubation for 15 minutes at RT, used in the FLIPR assay for the Ca²⁺ measurement. The Ca²⁺-dependent fluorescence is in this connection measured both before and after addition of the substances to be investigated (wavelength λₑₓ=488 nm, λₑₘ=540 nm). The quantification is carried out by measuring the highest fluorescence intensity (FC, Fluorescence Counts) over time.

### FLIPR-Assay:

The FLIPR protocol consists of 2 additions of substances. First of all the compounds to be tested (10 μM) are pipetted onto the cells and the Ca²⁺ inflow is compared with the control (capsaicin 10 μM) (% activation referred to the Ca²⁺ signal after addition of 10 μM capsaicin). After 5 minutes' incubation 100 nM capsaicin are added and the inflow of Ca²⁺ is again determined.

Desensitising agonists and antagonists lead to a suppression of the Ca²⁺ inflow. The % inhibition compared to the maximum achievable inhibition with 10 μM capsaicin is calculated.

Starting from the percentage displacement caused by different concentrations of the compounds of the general formula I to be tested, IC₅₀ inhibiting concentrations that produce a 50% displacement of capsaicin were calculated. K, values for the test substances were obtained by reconversion using the (Cheng, Prusoff; Biochem. Pharmacol. 22, 3099-3108, 1973) relationship.

### III. Formalin Test on Mice

The investigation to determine the antinociceptic action of the compounds according to the invention is carried out in the formalin test on male mice (NMRI, 20 bis 30 g body weight, Iffa, Credo, Belgium).

According to D. Dubuisson et al., Pain 1977, 4, 161-174, in the formalin test the first (early) phases (0 to 15 minutes after the formalin injection) and the second (late) phase (15 to 60 minutes after the formalin injection) are different. The early phase, which is a direct reaction to the formalin injection, represents a model of acute pain, whereas the late phase is regarded as a model of persistent (chronic) pain (T. J. Coderre et al., Pain 1993, 52, 259-285). The corresponding literature descriptions are hereby incorporated by way of reference and count as part of the disclosure.

The compounds according to the invention are investigated in the second phase of the formalin test, in order to obtain information on the effects of substances on chronic/inflammation pain.

The administration time of the compounds according to the invention before the formalin injection is selected depending on the type of administration of the compounds according to the invention. The intravenous administration of 10 mg/kg body weight of the test substances is carried out 5 minutes before the formalin injection. This is administered by a single subcutaneous formalin injection (20 μL, 1% aqueous solution) into the dorsal side of the right rear paw, so that a nociceptive reaction, manifested in significant licking and biting of the affected paw, is induced in freely moving experimental animals.

The nociceptive behaviour is then continuously assessed by observing the animals for an investigation period of 3 minutes in the (second) late phase of the formalin test (21 to 24 minutes after the formalin injection). The pain behaviour is quantified by counting the time in seconds during which the animals licked and bit the affected paw during the investigation period.

The comparison is made in each case with control animals, which receive instead of the compounds according to the invention a neutral vehicle (0.9% aqueous sodium chloride solution) before the formalin injection. Based on the quantification of the pain behaviour, the effect of the substance in the formalin test is measured as the change compared to the corresponding control.

After injection of substances that have an antinociceptive action in the formalin test, the described behaviour of the animals, i.e. licking and biting, is either reduced or raised.

The invention is illustrated hereinafter with the aid of some examples. These descriptions are simply exemplary and do not restrict the general concept of the invention.

### EXAMPLES

The term “equivalents” (“equiv.”) denotes amount of substance equivalents, “RT” denotes room temperature, “M” and “N” are concentration figures in mol/l, “aq.” denotes aqueous, “satd.” denotes saturated, “soln.” denotes solution and “conc.” denotes concentrated.

Further Abbreviations:

AcOH acetic acid
d days
BOP 1-benzotriazolyloxy-tris-(dimethylamino)-phosphonium hexafluorophosphate
Brine saturated aqueous sodium chloride solution (NaCl soln.)
DCC N,N′-dicyclohexylcarbodiimide
DCM dichloromethane

### DIPEA N,N-diisopropylethylamine

### DMF N,N-dimethylformamide

DMAP 4-dimethylaminopyridine
EDC N-(3-dimethylaminopropyl)-N′-ethyl-carbodiimide
EDCI N-ethyl-N′-(3-dimethylaminopropyl)-carbodiimide hydrochloride
EE ethyl acetate
Ether diethyl ether
EtOH ethanol
ges. satd.
h hour(s)
H₂O water

### HOBt N-hydroxybenzotriazole

LG leaving group
m/z mass to charge ratio
MeCN acetonitrile
MeOH methanol
min minutes
MS mass spectrometry
N/A not available
NEt₃ triethylamine
SC column chromatography on silica gel
THF tetrahydrofuran
vv volume ratio

The yields of the prepared compounds were not optimised.

All temperatures are uncorrected.

All starting substances that are not explicitly described were either commercially available (suppliers such as for example Acros, Avocado, Aldrich, Bachem, Fluke, Lancaster, Maybridge, Merck, Sigma, TCI, Oakwood, etc., can be searched for example in the Symyx® Available Chemicals Database of the company MDL, San Ramon, US, or in the SciFinder databank of the ACS, Washington, D.C., US) or their synthesis is already described in detail in the specialist literature (experimental procedures can be searched for example in the Reaxys® databank of the company Elsevier, Amsterdam, NL or in the SciFinder databank of the ACS, Washington, D.C., US) or can be prepared by conventional methods known to the person skilled in the art.

Silica gel 60 (0.04-0.063 mm) from the company E. Merck, Darmstadt, was used as stationary phase for the column chromatography.

The thin-layer chromatography investigations were carried out with HPTLC precoated plates, silica gel 60 F 254, from E. Merck, Darmstadt.

The mixture ratios of solvents, solvent systems or for chromatographic investigations are always given in volume/volume.

The analytical characterisation of all intermediate products and example compounds can be performed by ¹H-NMR spectroscopy. In addition mass spectrometry investigations can be carried out for all example compounds and selected intermediate products (MS, value of m/z for [M+H]⁺).

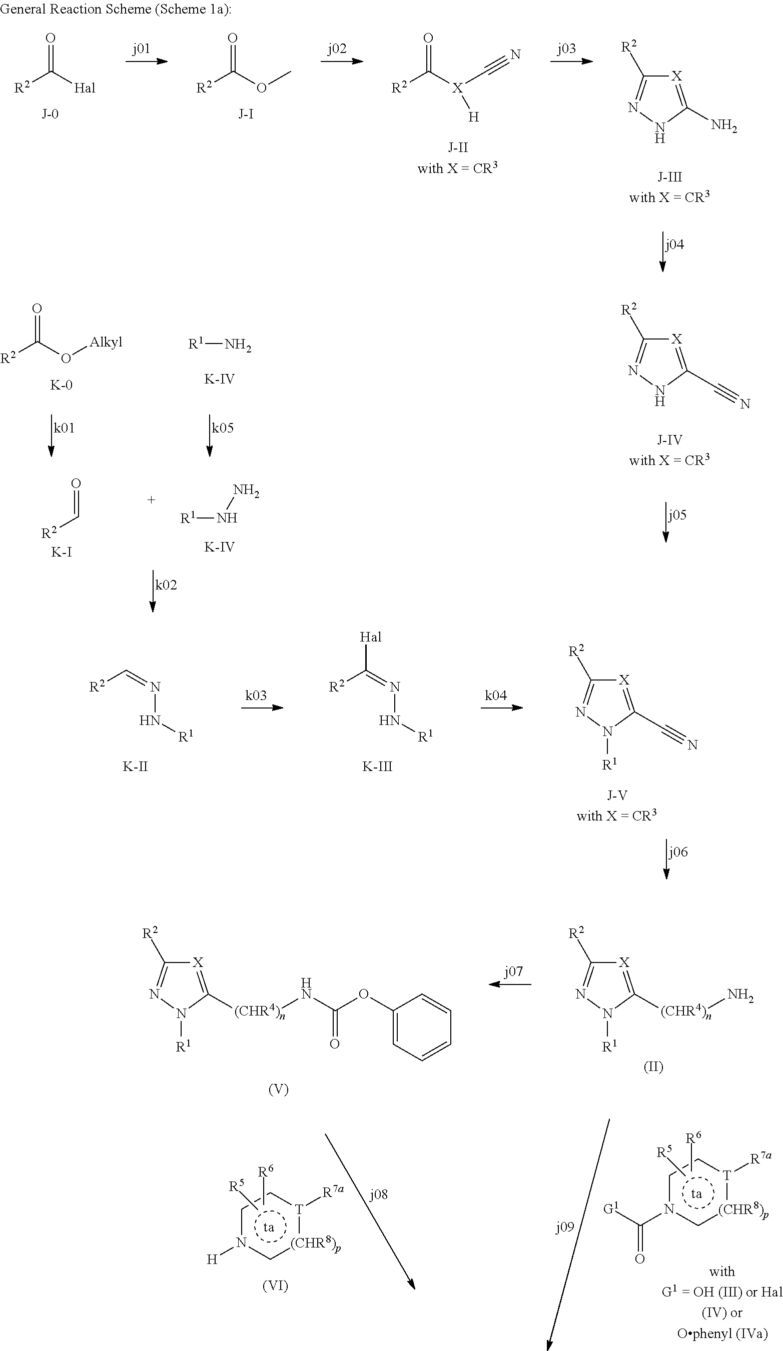

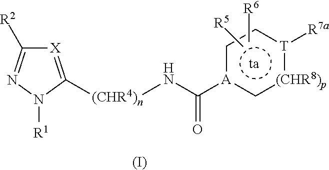

In step j01 an acid halide J-0, in which Hal preferably denotes Cl or Br, can be esterified to form the compound J-I using methanol, by means of methods known to the person skilled in the art.

In step j02 the methyl pivalate J-I can be converted into an oxoalkylnitrile J-II where X═CR³ by methods known to the person skilled in the art, such as for example by using an alkyl nitrile R³CH₂—CN, optionally in the presence of a base.

In step j03 the compound J-II can be converted into an amino-substituted pyrazolyl derivative J-III where X═CR³ by means of methods know to the person skilled in the art, such as for example using hydrazine hydrate with ring closure.

In step j04 the amino compound J-III can first of all be converted into a diazonium salt by means of methods known to the person skilled in the art, such as for example using nitrite, and this can be converted into a cyano-substituted pyraxolyl derivate J-IV where X═CR³ with the elimination of nitrogen by using a cyanide, optionally in the presence of a coupling reagent.

In step j05 the compound J-IV can be substituted in the N-position by means of methods known to the person skilled in the art, for example using a halide R¹ Hal, optionally in the presence of a base and/or a coupling reagent, where Hal is preferably Cl, Br or I, or by using a boronic acid B(OH)₂R¹ or a corresponding boronic acid ester, optionally in the presence of a coupling reagent and/or a base, and the compound J-V where X═CR³ can thereby be obtained. If R¹ is coupled via a hetero atom to the general formula (I) (where R¹ denotes for example the partial structure (T-1), in which o denotes 1 and Y can denote inter alia O, S, S(═O)₂ or NR¹²), then the substitution can be carried out by methods known to the person skilled in the art, for example with the aid of hydroxylamino-O-sulfonic acid followed by conversion into secondary and tertiary amines where Y═NR¹³. In the case where Y═O the substitution can be carried out by methods known to the person skilled in the art, for example with the aid of peroxy reagents and subsequent conversion to ether. In the case where Y═S(═O)₂ the substitution can take place for example by sulfonylation with sulphonyl chlorides. In the case where Y═S the formation can be carried out for example by reaction with disulfides or with sulfenyl chlorides or sulfenamides, but also by conversion to the mercaptan by means of methods known to the person skilled in art, and subsequent conversion to the thioether.

Alternatively, a second synthesis route is suitable for the preparation of the compound J-V where X═CR³, in which in the step k01 first of all an ester K-0 is reduced to the aldehyde K-I by means of methods known to the person skilled in the art, for example by using suitable hydrogenation reagents such as metal hydrides.

In step k02 the aldehyde K-I can then be converted with a hydrazine K-V, which can be obtained in step k05 by means of methods known to the person skilled in the art starting from the primary amine K-IV, to form the hydrazine K-II by methods known to the person skilled in the art, with the elimination of water.

In step k03 the hydrazine K-II can be halogenated, preferably chlorinated, by methods known to the person skilled in the art with retention of the double bond, for example using a chlorination reagent such as NCS, and the compound K-III can thereby be obtained.

In step k04 the hydrazonoyl halide K-III can be converted by methods known to the person skilled in the art, for example using a halogen-substituted nitrile with ring closure, to a cyano-substituted compound J-V with X═CR³.

In step j06 the compound J-V can be hydrogenated by methods known to the person skilled in the art, for example using a suitable catalyst such as palladium/activated charcoal or by using suitable hydrogenation reagents, and the compound (II) can thereby be obtained.

In step j07 the compound (II) can be converted to the compound (V) by methods known to the person skilled in the art, for example by the use of phenyl chloroformate, optionally in the presence of a coupling reagent and/or a base. Apart from the method illustrated here for preparing nonsymmetrical ureas using phenyl chloroformate, there are further methods known to the person skilled in the art, based on the use of optionally activated carboxylic acid derivates or isocyanates.

In step j08 the amine (VI) can be converted into the urea compound (I) (where A=N). This can be accomplished by a reaction with (V) by means of methods known to the person skilled in the art, optionally in the presence of a base.

In step j09 the amine (II) can be converted into the amide (I) (where A=CH or C). this can be accomplished for example by reaction with an acid halide, preferably a chloride of the formula (IV) by methods known to the person skilled in the art, optionally in the presence of a base, or by reaction with an acid of the formula (III), optionally in the presence of a suitable coupling reagent, for example HATU or CDI, optionally with the addition of a base. Furthermore the amine (II) can be converted to the amide (I) (where A=CH or C) by means of reactions with a compound (IVa) known to the person skilled in the art, optionally in the presence of at least one base.

### General Reaction Scheme (Scheme 1b):

To prepare the compounds (II) and (I) where X═N it is necessary to follow a third synthesis route according to the General Reaction Scheme 1b. The compounds (II) then obtained where X═N can afterwards be reacted further according to the aforedescribed steps j07-j09.

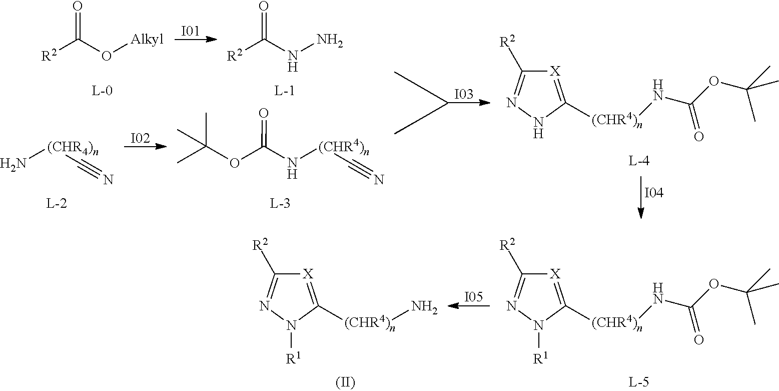

In step I01 a carbonic acid alkyl ester L-0, preferably a methyl or ethyl ester, can be converted with hydrazine hydrate by means of methods known to the person skilled in the art to form the hydrazide L-1.

In step I02 the amino-substituted nitrile L-2 or its salts can be converted with Boc-anhydride by means of methods known to the person skilled in the art to form the urethane L-3.

In step I03, L-1 and L-3 can be condensed in the presence of a base, preferably an alkali alcoholate, particularly preferably sodium methanolate, by means of methods known to the person skilled in the art to form the triazole L-4 where X═N.

In step I04 the compound L-4 where X═N can be substituted by means of methods known to the person skilled in the art in the N-position analogously to step j05 according to the General Reaction Scheme 1a by means of the methods described hereinbefore, and the compound L-5 where X═N can thereby be obtained.

In step I05 the ester group in L-4 can be eliminated in the presence of an acid, preferably trifluoroacetic acid or hydrochloric acid, by means of methods known to the person skilled in the art, and the amine (II) can thereby be obtained.

Compounds (I) where A=N can furthermore be prepared by means of a synthesis route according to the General Reaction Scheme 1c.

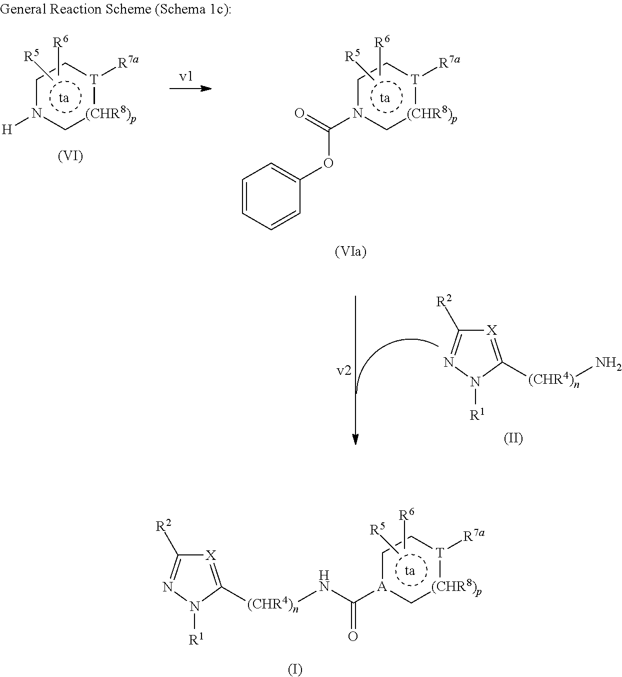

In step v1 the compound (VI) can be converted by means of methods known to the person skilled in the art, for example with the use of phenyl chloroformate, optionally in the presence of a coupling reagent and/or a base, to the compound (VIa).

Apart from the method illustrated here for preparing unsymmetrical ureas or using phenyl chloroformate, there are further methods known to the person skilled in the art, based on the use of optionally activated carboxylic acid derivates or isocyanates.

In step v2 the amine (II) can be converted by means of reactions known to the person skilled in the art with a compound (Via) to the urea (I) (where A=N) optionally in the presence of at least one base.

The methods known to the person skilled in the art for carrying out the reaction steps j01 to j09 as well as k01 to k05 and I01 to I05 and also v1 and v2 can be obtained from standard text books on organic chemistry, such as for example J. March, Advanced Organic Chemistry, Wiley & Sons, 6th edition, 2007; F. A. Carey, R. J. Sundberg, Advanced Organic Chemistry, Parts A and B, Springer, 5th edition, 2007); author's collected works, compendium Organic Synthetic Methods, Wiley & Sons. In addition to this further methods as well as literature references can be obtained from the usual databanks, such as for example the Reaxys® Databank of Elsevier, Amsterdam, NL or the SciFinder® Databank of the American Chemical Society, Washington, US.

### SYNTHESIS OF INTERMEDIATE PRODUCTS

### 1. Synthesis of 3-tert-butyl-1-methyl-1H-pyrazol-5-yl-methanamine (Step j01-j06)

Step j01: Pivaloyl chloride (J-0) (1 equiv., 60 g) was added dropwise within 30 min to a solution of MeOH (120 mL) at 0° C. and stirred for 1 hr at room temperature. After adding water (120 mL) the separated organic phase was washed with water (120 mL), dried over sodium sulphate and co-distilled with dichloromethane (150 mL). The liquid product J-I could be obtained in 98.6% purity (57 g).

Step j02: NaH (50% in paraffin oil) (1.2 equiv., 4.6 g) was dissolved in 1,4-dioxane (120 mL) and stirred for a few minutes. Acetonitrile (1.2 equiv., 4.2 g) was added dropwise within 15 min and the mixture was stirred for a further 30 min. The methyl pivalate (J-I) (1 equiv., 10 g) was added dropwise within 15 min and the reaction mixture was refluxed for 3 h. After complete conversion the reaction mixture was poured into iced water (200 g), acidified to pH 4.5, and extracted with dichloromethane (12×250 mL). The combined organic phases were dried over sodium sulphate, distilled, and after recrystallisation from hexane (100 mL) 5 g of the product (J-II) (51% yield) was obtained as a brown solid substance.

Step j03: 4,4-dimethyl-3-oxopentane nitrile (J-II) (1 equiv., 5 g) was taken up at room temperature in EtOH (100 mL), hydrazine hydrate (2 equiv., 4.42 g) was added, and the mixture was refluxed for 3 h. The residue obtained after distilling off the EtOH was taken up in water (100 mL) and extracted with EE (300 mL). The combined organic phases were dried over sodium sulphate, the solvent was removed in vacuo and the product (JAI) (5 g, 89% yield) was obtained as a pale red solid after recrystallisation from hexane (200 mL).

Step j04: 3-tert-butyl-1H-pyrazol-5-amine (J-III) (1 equiv., 40 g) was dissolved in dilute HCl (120 mL HCl in 120 mL water) and NaNO₂(1.03 equiv., 25 g in 100 mL) was added dropwise over 30 min. After stirring for 30 min the reaction mixture was neutralised with Na₂CO₃. A diazonium salt obtained by reacting KCN (2.4 equiv., 48 g), water (120 mL) and CuCN (1.12 equiv., 31 g) was added dropwise within 30 min to the reaction mixture, which was then stirred for a further 30 min at 75° C. After complete conversion the reaction mixture was extracted with EE (3×500 mL), the combined phases were dried over sodium sulphate and the solvent was removed in vacuo. The column chromatography purification (SiO₂, 20% EE/Hexane) of the residue yielded a white solid (J-IV) (6.5 g, 15.1% yield).

Step j05 (Method 1):

3-tert.-butyl-1H-pyrazole-5-carbonitrile (J-IV) (10 mmol) was added at room temperature to a suspension of NaH (60%) (12.5 mmol) in DMF (20 mL) while stirring. After stirring for 15 min methyl iodide (37.5 mmol) was added at room temperature to the reaction mixture. After stirring for 30 min at 100° C. water (150 mL) was added to the reaction mixture and the mixture was extracted with dichloromethane (3×75 mL). The combined organic extracts were washed with water (100 mL) and satd. NaCl solution (100 mL) and dried over magnesium sulphate. After removing the solvent in vacuo the residue was purified by column chromatography (SiO₂, various mixtures of EE and cyclohexane as solvent) and the product J-V was obtained.

Step j06:

Method 1:

J-V was dissolved together with palladium on charcoal (10%, 500 mg) and conc. HCl (3 mL) in MeOH (30 mL) and exposed to a hydrogen atmosphere for 6 hours at room temperature. The reaction mixture was filtered through Celite and the filtrate was concentrated by evaporation in vacuo. The residue was purified by means of flash chromatography (SiO₂, EE) and the product (II) was thereby obtained.

Method 2:

J-V was dissolved in THF (10 mL) and BH₃.S(CH₃)₂ (2.0 M in THF, 3 mL, 3 equiv.) was added. The reaction mixture was heated under reflux for 8 hours, aq. 2 N HCl (2 N) was added, and the reaction mixture was refluxed for a further 30 min. Aq. NaOH solution (2N) was added to the reaction mixture and washed with EE. The combined organic phases were washed with satd. aq. NaCl solution and dried over magnesium sulphate. The solvent was removed in vacuo and the residue was purified by column chromatography (SiO₂, various mixtures of dichloromethane and methanol as solvent) and the product (II) (3-tert-butyl-1-methyl-1H-pyrazol-5-yl)methanamine) was thereby obtained.

Alternatively step j05 can also be carried out as follows (Method 2):

### Step j05 (Method 2):

A mixture of 3-tert-butyl-1H-pyrazole-5-carbonitrile (J-IV) (10 mmol), a boronic acid B(OH)₂R¹ or a corresponding boronic acid ester (20 mmol) and copper (II) acetate (15 mmol) is added to dichloromethane (200 mL). Pyridine (20 mmol) is added while stirring at room temperature, and the resultant mixture is stirred for 16 h. After removing the solvent in vacuo the residue obtained is purified by column chromatography (SiO₂, various mixtures of EE and cyclohexane as solvent) and the product J-V is thereby obtained.

The following further intermediate products were prepared in this way (steps j01-j06):
3-tert-butyl-1-(4-chlorophenyl)-1H-pyrazol-5-yl)methanamine
(3-tert-butyl-1-(3-chloro-4-fluorophenyl)-1H-pyrazol-5-yl)methanamine

### 2. Synthesis of 1(3-chlorophenyl)-3-(trifluoromethyl)-1H-pyrazol-5-yl-methanamine (Steps k01-k05 and j06)

### Step k01:

LAIH (lithium aluminium hydride) (0.25 equiv., 0.7 g) was dissolved in dry diethyl ether (30 mL) under a protective gas atmosphere and stirred for 2 hours at room temperature. The suspension obtained was taken up in diethyl ether (20 mL). Ethyl-2,2,2-trifluoracetate (K-0) (1 equiv., 10 g) was taken up in dry diethyl ether (20 mL) and added dropwise at −78° C. over a period of 1 h to the suspension. The suspension was then stirred for a further 2 h at −78° C. EtOH (95%) (2.5 mL) was now added dropwise, and the reaction mixture was heated to room temperature and added to iced water (30 mL) together with concentrated H₂SO₄ (7.5 mL). The organic phase was separated, concentrated by evaporation in vacuo, and the reaction product K-I was used directly in the next reaction step k02.

### Step k05:

3-chloroaniline (K-IV) (1 equiv., 50 g) was dissolved at −5 to 0° C. in concentrated HCl (300 mL) and stirred for 10 min. A mixture of NaNO₂ (1.2 equiv., 32.4 g), water (30 mL), SnCl₂.2H₂O (2.2 equiv., 70.6 g) and concentrated HCl (100 mL) was added dropwise over a period of 3 h while maintaining the temperature constant. After stirring for a further 2 h at −5 to 0° C., the reaction mixture was adjusted with NaOH solution to pH 9 and extracted with EE (250 mL). The combined organic phases were dried over magnesium sulphate and the solvent was removed in vacuo. The column chromatography purification (SiO₂, 8% EE/hexane) yielded 40 g (72% yield) of (3-chlorophenyl)hydrazine (K-IV) as a brown oil.

### Step k02:

The aldehyde (K-I) (2 equiv., 300 mL) obtained from k01 and (3-chlorophenyl)hydrazine (K-IV) (1 equiv., 20 g) were added to EtOH (200 mL) and refluxed for 5 h. The solvent was removed in vacuo, the residue was purified by column chromatography (SiO₂, hexane) and the product (25 g, 72% yield) K-II was obtained as a brown oil.

### Step k03:

The hydrazine K-II (1 equiv., 25 g) was dissolved in DMF (125 mL). N-chlorosuccinimide (1.3 equiv., 19.5 g) was added in portions at room temperature within 15 min and the mixture was stirred for 3 h. The DMF was distilled off and the residue was taken up in EE. The EE was removed in vacuo, the residue obtained was purified by column chromatography (SiO₂, hexane) and the product K-III (26.5 g, 92% yield) was obtained as a pink-coloured oil.

### Step k04:

The hydrazonoyl chloride K-III (1 equiv., 10 g) was taken up in toluene (150 mL) at room temperature and 2-chloroacrylonitrile (2 equiv., 6.1 mL) and TEA (2 equiv., 10.7 mL) were added. This reaction mixture was stirred for 20 h at 80° C. The reaction mixture was then diluted with water (200 mL) and the phases were separated. The organic phase was dried over magnesium sulphate and the sulphate was removed in vacuo. The residue was purified by means of column chromatography (SiO₂, 5% EE/hexane) and the product (5.5 g, 52% yield) was obtained as a white solid J-V.

### Step j06 (Method 3):

The carbonitrile J-V (1 equiv., 1 g) was dissolved in methanolic ammonia solution (150 mL, 1:1) and hydrogenated in an H-cube (10 bar, 80° C., 1 mL/min, 0.25 mol/L). After removing the solvent in vacuo (1-(3-chlorophenyl)-3-(trifluoromethyl)-1H-pyrazol-5-yl)methanamine (II) was obtained as a white solid (0.92 g, 91% yield).

The synthesis of the following further intermediate products can be carried out in a similar way according to one of the processes described hereinbefore:
(1-m-tolyl-3-(trifluoromethyl)-1H-pyrazol-5-yl)methanamine
(1-pentyl-3-(trifluoromethyl)-1H-pyrazol-5-yl)methanamine

### 3. Preparation of Selected Compounds of the General Formula (VI)

3.1 Preparation of Compounds of the General Formula (Vi) where T=N and A=N

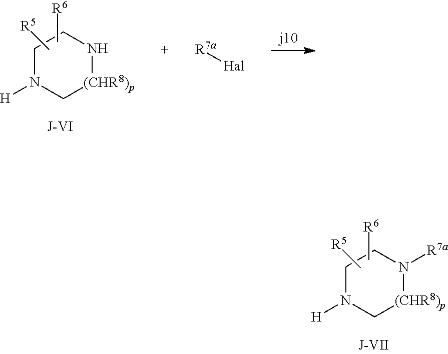

In step j10 the compound J-VI can be monosubstituted in the N-position by means of methods known to the person skilled in the art, for example by using a halide R^{7a}, optionally in the presence of a base and/or a coupling reagent, where Hal is preferably Cl, Br or I.

### 3.1.1 Preparation of 1-(3-chloropyridin-2-yl)piperazine

A solution of piperazine (according to J-VI) (71 mmol, 6.1 g) and 2,3-dichloropyridine (6.75 mmol, 1 g) was dissolved in 1-butanol (55 mL) and refluxed for 3 h. After concentrating the reaction mixture by evaporation in vacuo, the residue was taken up in EE (20 mL) and washed with water (2×20 mL). The combined organic phases were dried over magnesium solphate and concentrated by evaporation in vacuo. The product J-VII is obtained as a yellowish oil (1.25 g, yield 94%).

### 3.1.2 Preparation of 1-(1-(4-fluorophenyl)ethyl)piperazine

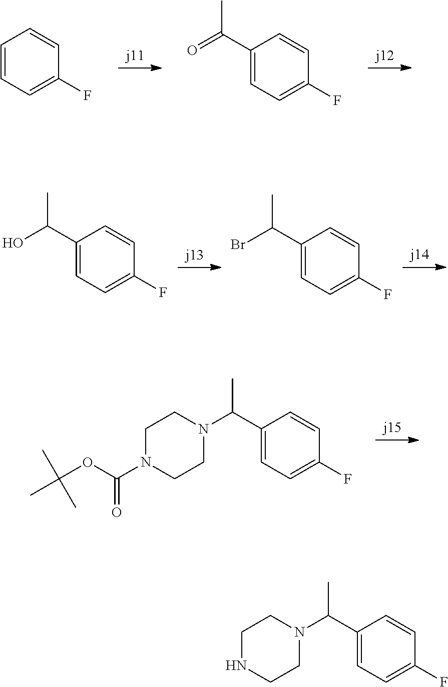

Step j11: Acetic anhydride (1.2 equiv., 320 g) was added dropwise at 4° C. over a period of 1 h to a mixture of AlCl₃ (1.2 equiv., 416 g) and fluorobenzene (1 equiv., 250 g) and stirred for 2 h. The reaction mixture was added to a solution of iced water (2.5 kg) and HCl (250 mL), and the organic phase was separated and distilled at 150° C./10 mm. 1-(4-fluorophenyl)ethanone was obtained in 30% yield (108 g) as a pale yellow liquid.

Step j12: 1-(4-fluorophenyl)ethanone (1 equiv., 25 g) was dissolved in MeOH (200 mL), NaBH₄ (1 equiv., 6.5 g) was added in portions at 4° C. within 45 min, and the mixture was stirred for 30 min. After adding water (100 mL) the reaction mixture was extracted with EE (3×100 mL). The combined organic phases were dried over sodium sulphate. After removing the solvent in vacuo the liquid product 1-(4-fluorophenyl)ethanol was obtained (25 g, 99% yield).

Step j13: 1-(4-fluorophenyl)ethanol (1 equiv., 25 g) was taken up in dichloromethane (150 mL), and PBr3 (0.7 equiv., 12 mL) was added dropwise at 4° C. over a period of 20 min. The reaction mixture was stirred for 4 h at room temperature, then added to iced water (200 g) and extracted with dichloromethane (3×100 mL). The combined organic phases were dried over sodium sulphate, concentrated by evaporation in vacuo, and the liquid product (1-bromoethyl)-4-fluorobenzene was obtained (30 g, 83% yield).

Step j14: 1-(1-bromoethyl)-4-fluorobenzene (1.2 equiv., 18 g) and K₂CO₃ (2 equiv., 22 g) were added at room temperature to a solution of tert.-butyl-piperazine-1-carboxylate (1 equiv., 15 g) in DMF (15 mL) and stirred for 2 h. After adding cold water (20 mL) the mixture was extracted with hexane (10×60 mL). The combined organic phases were dried over sodium sulphate and acidified with 10% HCl. The precipitate was suction filtered, washed with hexane (3×100 mL), adjusted to pH9 with K₂CO₃ and re-extracted with hexane (5×100 mL). The combined organic phases were dried over sodium sulphate. The removal of the solvent in vacuo yielded a viscous product (tert-butyl 4-(1-(4-fluorophenyl)ethyl)piperazine-1-carboxylate) (12.5 g, 50% yield).

Step j15: tert-butyl 4-(1-(4-fluorophenyl)ethyl)piperazine-1-carboxylate (16.2 mmol, 5 g) was dissolved in MeOH (100 mL), isopropanolic HCl (106 mL) was added dropwise at 4° C., and the mixture was stirred for 12 h at room temperature. After removing the solvent in vacuo the residue was taken up with diethyl ether (100 mL). The precipitate formed was suction filtered, washed with diethyl ether (2×50 mL), and the product 1-(1-(4-fluorophenyl)ethyl)piperazine (4.59 g, 100% yield) was obtained as a white solid.

### 4. Preparation of Selected Carbamic Acid Phenyl Esters of the General Formula (V)

### 4.1 Preparation of phenyl (3-tert-butyl-1-(3-chlorophenyl)-1H-pyrazol-5-yl)methyl carbamate (Used for the Synthesis of Examples 1, 2, 5, 22 and can be Used for the Synthesis of Example 20)

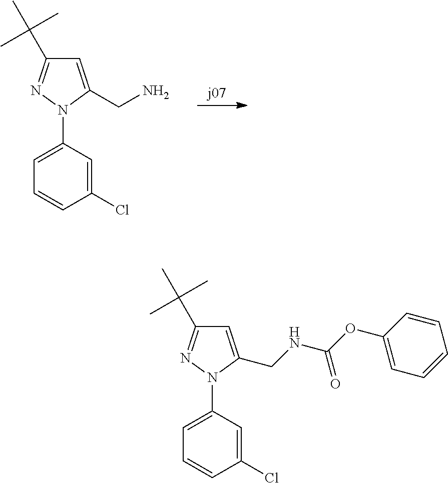

Step j07: Potassium carbonate (9.16 g, 66 mmol, 3.5 equiv.) was added to a solution of (3-tert-butyl-1-(3-chlorophenyl)-1H-pyrazol-5-yl)methanamine (5 g, 18 mmol, 1 equiv.) in DMF (25 mL) and the resultant reaction mixture was cooled to 0° C. Phenyl chloroformate (3.28 g (2.65 mL), 20 mmol, 1.1 equiv.) was then added dropwise over a period of 15 minutes and the mixture was stirred for a further 15 minutes at 0° C. The mixture was filtered, and the filtrate was diluted with cold water (100 mL) and then extracted with ethyl acetate (3×25 mL). The combined organic phases were washed with saturated sodium chloride solution (100 mL), dried over Na₂SO₄, and the solvent was evaporated under reduced pressure. The crude product was purified by column chromatography (SiO₂, EE/n-hexane 1/9, v/v) and the desired product was obtained as a white solid (3.2 g, 45% yield).

The synthesis of the following further carbamic acid phenyl esters can be carried out in a similar manner in accordance with the processes described hereinbefore:
Phenyl (3-tert-butyl-1-(4-chlorophenyl)-1H-pyrazol-5-yl)methyl carbamate (for the synthesis of Example 4);
Phenyl (3-tert-butyl-1-(3-chloro-4-fluorophenyl)-1H-pyrazol-5-yl)methyl carbamate (for the synthesis of Example 6);
Phenyl (3-tert-butyl-1-(pyridine-2-yl)-1H-pyrazol-5-yl)methyl carbamate (for the synthesis of Example 7);
Phenyl (1-(3-chlorophenyl)-3-cyclopropyl-1H-pyrazol-5-yl)methyl carbamate (for the synthesis of Example 10);
Phenyl (3-tert-butyl-1-(3-chlorophenyl)-1H-1,2,4-triazol-5-yl)methyl carbamate (for the synthesis of Examples 43 and 44).

### 4.2 Preparation of phenyl (1-(3-chlorophenyl)-3-(trifluoromethyl)-1H-pyrazol-5-yl)methyl Carbamate (for the Synthesis of Examples 3, 14-19 and 23-42)

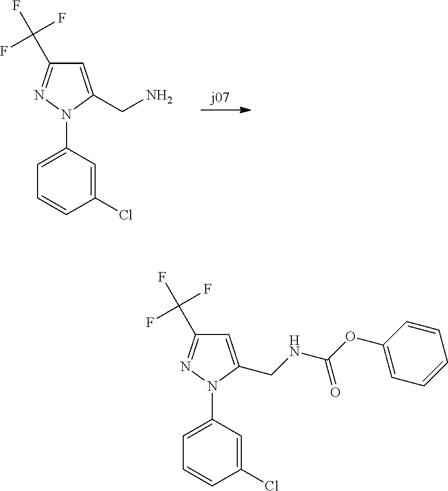

Step j07: Phenyl chloroformate (1.28 mL, 10.2 mmol, 1.12 equiv.) and triethylamine (1.5 mL, 10.9 mmol, 1.2 equiv.) were added at room temperature while stirring to a solution of (1-(3-chlorophenyl)-3-(trifluoromethyl)-1H-pyrazol-5-yl)methanamine (2.5 g, 9.1 mmol, 1 equiv.) in dichloromethane (50 mL). After stirring for 12 h at room temperature the reaction mixture was extracted with Na₂CO₃ solution (1×25 mL) and dichloromethane (2×25 mL). The combined organic phases were dried over magnesium sulphate and concentrated by evaporation in vacuo. The column chromatography purification (SiO₂, cyclohexane/diethyl ether 1/1, v/v) of the crude product yielded a white solid (2.9 g, 81% yield).

The synthesis of the following further carbamic acid phenyl esters can be carried out in a similar way in accordance with the processes described hereinbefore:
Phenyl (1-m-tolyl-3-(trifluoromethyl)-1H-pyrazol-5-yl)methyl carbamate (for the synthesis of Example 8);
Phenyl (1-(3-chlorophenyl)-4-methyl-3-(trifluoromethyl)-1H-pyrazol-5-yl)methyl carbamate (for the synthesis of Example 9);
Phenyl (1-(4-methoxybenzyl)-3-(trifluoromethyl)-1H-pyrazol-5-yl)methyl carbamate (for the synthesis of Example 11);
Phenyl (1-pentyl-3-(trifluoromethyl)-1H-pyrazol-5-yl)methyl carbamate (for the synthesis of Example 12);
Phenyl (1-(tetrahydro-2H-pyran-4-yl)-3-(trifluoromethyl)-1H-pyrazol-5-yl)methyl carbamate (for the synthesis of Example 13);
Phenyl (1-hexyl-3-(trifluoromethyl)-1H-1,2,4-triazol-5-yl)methyl carbamate (for the synthesis of Example 45).

### 5. Preparation of Further Selected Pyrazole Derivatives of the General Formula (II)

### 5.1 Preparation of 1-(3-chlorophenyl)-4-methyl-3-(trifluoromethyl)-1H-pyrazol-5-yl)methanamine (for the Synthesis of Example 9)

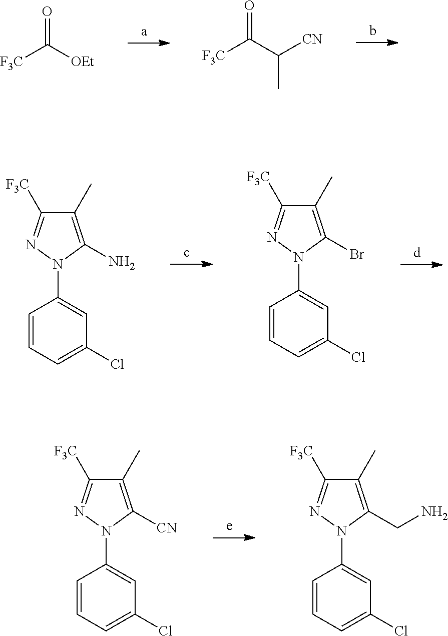

Step a: n-BuLi (1.6 molar, 258.3 mL, 380 mmol, 2.2 equiv.) was added dropwise at −20° C. over a period of 2 h to a solution of diisopropylamine (57 mL, 404 mmol, 2.3 equiv.) in THF (400 mL). After cooling the reaction mixture to −75° C. ethyl 2,2,2 trifluoroacetate (25 g, 170 mmol) in THF (200 mL) was added dropwise within 2 h and the mixture was stirred for 1 h at −75° C. and for a further hour at room temperature. After complete conversion the reaction was quenched with iced water (700 mL) and the solvent was distilled off. The residue obtained was washed with dichloromethane (3×400 mL), acidified with 30% HCl solution, and the product was extracted with diethyl ether (3×300 mL). The combined organic phases were dried over sodium sulphate and concentrated by evaporation in vacuo. The product was obtained as a colourless oil (17 g, 64% yield).

Step b: 4,4,4-trifluoro-2-methyl-3-oxobutanenitrile (10 g, 66 mmol, 1 equiv.) was taken up in ethanolic HCl solution (300 mL) and 3-chlorophenylhydrazine (9.43 g, 66 mmol, 1 equiv.) was added. After stirring for 2 h under reflux the solvent was removed in vacuo and the resultant residue was taken up in water (200 mL). After adjusting the pH to 12 by means of 1 N NaOH a solid was obtained by filtration. This was taken up in EtOAc (200 mL) and the solution was dried over sodium sulphate and concentrated by evaporation in vacuo. The product was obtained as a red solid (12 g, 65% yield).

Step c: Copper bromide (11.33 g, 51.1 mmol, 1.2 equiv.) was taken up in acetonitrile (176 mL) and heated to 150° C. After adding n-butyl nitrite (6.59 g (7.47 mL), 63 mmol, 1.5 equiv.) the 1-(3-chlorophenyl)-4-methyl-3-(trifluoromethyl)-1H-pyrazol-5-amine (11.75 g, 42 mmol) obtained in step b was added dropwise in acetonitrile (176 mL) over a period of 30 min and the mixture was stirred for 15 min at 150° C. The acetonitrile was distilled of and the resultant residue was taken up in iced water (300 mL) and extracted with EtOAc (5×100 mL). The combined organic phases were dried over sodium sulphate, concentrated by evaporation in vacuo, and the crude product obtained was purified by column chromatography (SiO₂, n-hexane). The product was obtained as a red oil (16 g) and used directly in the next step.

Step d: The product from step c (13 g, 38 mmol, 1 equiv.) was taken up in NMP (130 mL), and copper cyanide (6.8 g, 76 mmol, 2 equiv.) and sodium iodide (100 mg, catalytic) were added and the mixture was stirred for 8 h at 180° C. The reaction mixture was diluted with water (200 mL) and extracted with EtOAc (5×100 mL). The combined organic phases were washed with cold water (5×50 mL), dried over sodium sulphate and concentrated by evaporation in vacuo. After column chromatography purification (SiO₂, EtOAc/n-hexane 2/98, v/v) the product was obtained as a yellow solid (8 g).

Step e: 1-(3-chlorophenyl)-4-methyl-3-(trifluoromethyl)-1H-pyrazole-5-carbonitrile (5 g, 17 mmol) was dissolved in dry THF (30 mL). Borane-THF in THF (70 mL) was added dropwise at 5° C. within 30 min. The reaction mixture was slowly heated to 50° C. and stirred for 12 h. After complete conversion the reaction mixture was acidified with conc. HCl at 5° C. and stirred for 2 h at room temperature. The reaction mixture was then adjusted alkaline to pH˜12 with 10% NaOH and the product was extracted with EtOAc (5×50 mL). The combined organic phases were dried over sodium sulphate and concentrated by evaporation in vacuo. The solid obtained was washed with a 10% diethyl ether/n-hexane mixture and dried. The product was obtained as a white solid (3 g, 59% yield).

### 5.2 Preparation of (1-(3-chlorophenyl)-3-cyclopropyl-1H-pyrazol-5-yl)methanamine hydrochloride (for the Synthesis of Example 10)

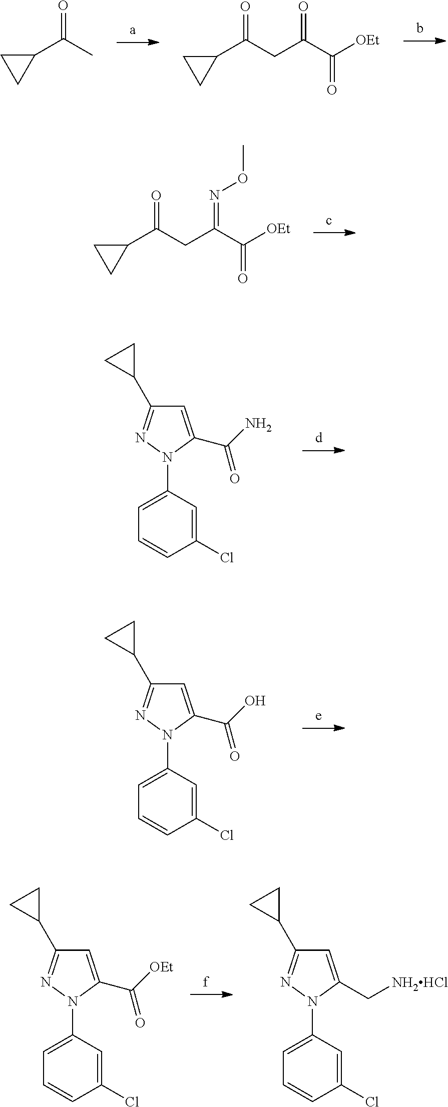

Step a: Diethyl oxalate (0.92 mL, 6.85 mmol, 1 equiv.) was added at RT to a freshly prepared sodium methanolate solution (prepared by dissolving sodium (1 g, 8.2 mmol, 1.2 equiv.) in EtOH (30 mL)), and cyclopropyl methyl ketone (0.74 mL, 7.5 mmol, 1.1 equiv.) was then added dropwise at 0° C. The reaction mixture was slowly heated to RT and stirred for a further 3 h. Ice-cold water (10 mL) was added and the EtOH was distilled off under reduced pressure. The remaining aqueous phase was diluted with 2N hydrochloric acid (15 mL) and extracted with diethyl ether (2×25 mL). The combined organic phases were washed with saturated sodium chloride solution, dried over Na₂SO and concentrated under reduced pressure. A pale brown liquid was obtained as product (400 mg, 31% yield).

Step b: Methoxyl amine hydrochloride (30% solution in water, 0.4 mL, 0.651 mmol, 1.2 equiv.) was added at room temperature to the product obtained from step a (200 mg, 0.543 mmol, 1 equiv.) in EtOH (8 mL), and the reaction mixture was stirred for 1 h at RT. EtOH was evaporated under reduced pressure and the remaining aqueous phase was extracted with EA (15 mL). The organic phase was washed with water (10 mL), saturated sodium chloride solution (10 mL), dried over Na₂SO₄ and concentrated under reduced pressure. A pale yellow liquid was obtained as product (180 mg, 79% yield).

Step c: A mixture of the product obtained from step b (1.1 g, 5.164 mmol, 1 equiv.) and 3-chlorophenylhydrazine hydrochloride (1.84 g, 10.27 mmol, 2 equiv.) was added to a mixture of acetic acid (20 mL) and 2-methoxyethanol (10 mL), and the resultant mixture was heated at 105° C. for 3 h. The solvent was distilled off and the remaining mixture was extracted with EA (60 mL). The organic phase was washed with water (10 mL), saturated sodium chloride solution (10 mL), dried over Na₂SO₄ and concentrated under reduced pressure. After column chromatography (SiO₂, EA/petroleum ether, 4/96, v/v) a pale brown semi-solid substance was obtained as the desired product (1.15 g, 77% yield).

Step d: LiOH (1.08 g, 25.71 mmol, 3 equiv.) was added at 0° C. to a solution of the product obtained from step c (2.5 g, 8.62 mmol, 1 equiv.) in THF-MeOH-water (15 mL-9 mL-3 mL) and the mixture was stirred for 2 h at RT. The solvents were distilled off and the residue was acidified with 2 N hydrochloric acid (1.2 mL) to pH˜3.). The acidic solution was then extracted with EA (2×60 mL), the combined organic phases were washed with water (10 mL), saturated sodium chloride solution (10 mL), dried over Na₂SO₄ and concentrated under reduced pressure. A white solid was obtained (1.4 g, 62% yield).

Step e: Pyridine (0.25 mL, 3.2 mmol, 0.6 equiv.) and di-tert-butyl dicarbonate (1.4 mL, 6.37 mmol, 1.2 equiv.) were added at 0° C. to a solution of the product obtained from step d (1.4 g, 5.34 mmol, 1 equiv.) in 1,4-dioxane (30 mL) and the resultant mixture was stirred for 30 min at 0° C. Ammonium bicarbonate (0.84 g, 10.63 mmol, 2 equiv.) was added at 0° C. and the mixture was stirred overnight at RT and then diluted with water (10 mL). The aqueous phase was extracted with EA (2×30 mL). The organic phase was washed with 2N HCl (20 mL), water (10 mL), saturated sodium chloride solution (10 mL), dried over Na₂SO₄ and concentrated under reduced pressure. After column chromatography (SiO₂, EA/petroleum ether 16:84, v/v) a white solid was obtained (1 g, 72% yield).

Step f: BH₃.DMS (1.44 mL, 15.32 mmol, 2 equiv.) was added at 0° C. to a solution of the product obtained from step e (2 g, 7.66 mmol, 1 equiv.) in THF (25 mL) and the mixture was then heated for 3 h at 70° C. The reaction mixture was then cooled to 0° C. and MeOH (15 mL) was added, following which the mixture was refluxed for 1 h. The mixture was then cooled to RT and the solvent was distilled off under reduced pressure. The residue was dissolved in ether (15 mL), cooled to 0° C., and an HCl solution in 1,4-dioxane (3 mL) was then added (to pH ˜4). The solid that precipitated out was then filtered off and washed with diethyl ether (5 mL) and the hydrochloride salt was thus obtained as a white solid (600 mg, 28% yield).

### 5.3 Preparation of (3-tert-butyl-1-(pyridine-2-yl)-1H-pyrazol-5-yl)methanamine (for the Synthesis of Example 7)

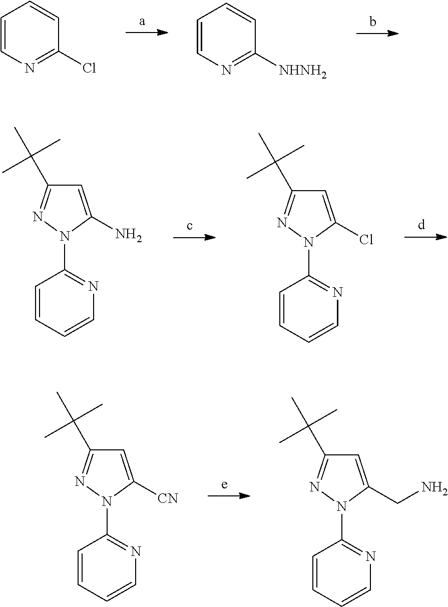

Step a: Hydrazine hydrate (132 mL) was added to a solution of 2-chloropyridine (20 g, 170 mmol) in EtOH (100 mL) and the reaction mixture was refluxed for 15 h. The course of the reaction was followed by thin-layer chromatography (40% EA in n-hexane, R_{f}˜0.1). After completion of the reaction the ethanolic hydrazine hydrochloride was completely distilled off at 100° C. and the residue was taken up in DCM (500 mL) and washed with saturated Na₂CO₃ solution (100 mL). The organic phase was dried over Na₂SO₄ and concentrated under reduced pressure. The crude product (11 g) having a low melting point was used directly in the next stage without further purification.

Step b: 4,4-dimethyl-3-oxopentane nitrile (11.3 g, 90 mmol, 0.9 equiv.) was added in portions to a solution of the product obtained from step a (11 g) in EtOH (110 mL), followed by a catalytically effective amount of HCl. The mixture was heated to 100° C. and then refluxed for 6 h. EtOH was distilled off and the residue was taken up in water (200 mL) and extracted with EA (2×100 mL). The combined organic phases were dried over Na₂SO₄ and concentrated under reduced pressure. After column chromatography (SiO₂, EA/n-hexane 1:9, v/v) a white solid was obtained (18 g).

Step c: Copper chloride (12.3 g, 90 mmol, 5 equiv.) was added to a solution of the product obtained from step b (4 g, 10 mmol) in acetonitrile (80 mL), and a solution of tert-butyl nitrite (2.8 (3.3 mL), 23 mmol, 1.5 equiv.) in acetonitrile (40 mL (total 120 mL)) was then added dropwise over a period of 10 minutes and the mixture was then stirred for a further 5 h at RT. Acetonitrile was distilled off, the residue was taken up in water (100 mL) and the mixture was then extracted with EA (2×200 mL). The combined organic phases were dried over Na₂SO₄ and concentrated under reduced pressure. After column chromatography (SiO₂, EA/n-hexane 4:96, v/v) a pale yellowish oil was obtained as the desired product (2.1 g, 48% yield).

Step d: Copper cyanide (1.56 g, 17 mmol, 2 equiv.) was added in portions to a stirred solution of the product obtained from step c (2.1 g, 8 mmol) in NMP (21 mL) followed by a catalytically active amount of NaI. The mixture was then heated to 180° C. and stirred for 4 h at this temperature. The mixture was then diluted with EA, filtered through Celite, and the filtrate was washed with cold water (50 mL). The organic phase was dried over Na₂SO₄ and concentrated under reduced pressure. After column chromatography (SiO₂, EA/n-hexane 8:92, v/v) a white solid was obtained (800 mg, 40% yield).

Step e: Raney nickel was added in a catalytically active amount to a solution of the product obtained from step d (1.5 g, 6 mmol) in MeOH (20 mL) and a hydrogenation with hydrogen was then carried out (1 h at 60 psi). The course of the reaction was followed by thin-layer chromatography (EN n-hexane 15:85, R_{f}˜0.1). After completion of the reaction the mixture was filtered through Celite and washed with MeOH. The filtrate was concentrated by evaporation and the residue was purified by column chromatography (SiO₂, EA/n-hexane 6/94, v/v). The product was obtained as a cream-coloured oil (1.4 g, 97% yield).

### 5.4 Preparation of (1-(4-methoxybenzyl)-3-(trifluoromethyl)-1H-pyrazol-5-yl)methanamine (for the Synthesis of Example 11)

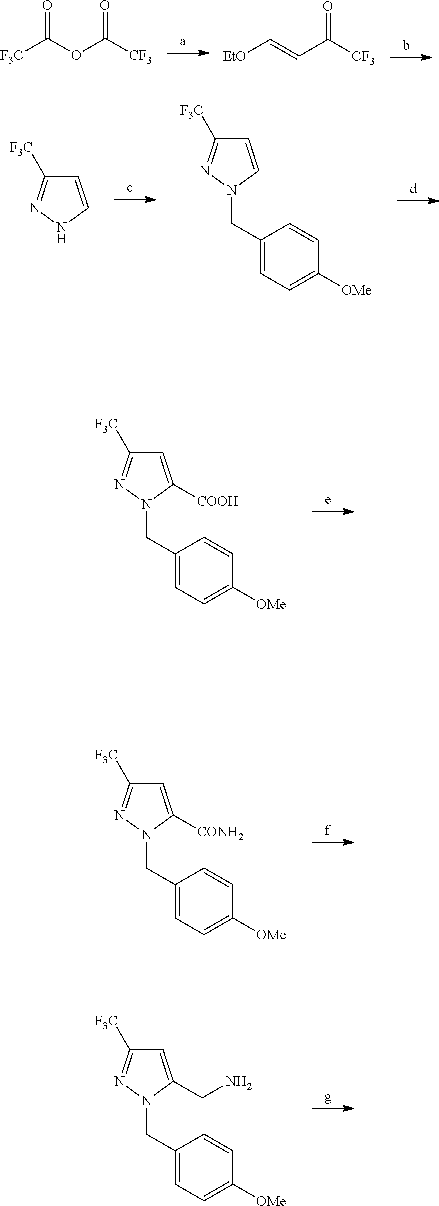

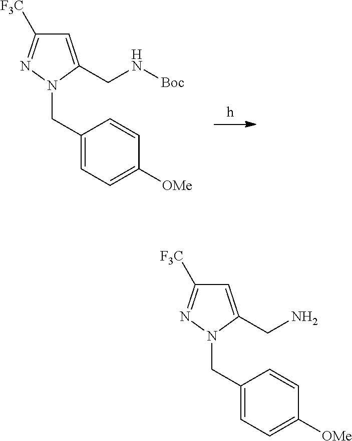

Step a: 4-dimethylaminopyridine (4.25 g, 34 mmol, 0.01 equiv.) was added to DCM (3000 mL) and cooled to −10° C. Trifluoroacetic anhydride (765 g (510 mL), 3200 mmol, 1.05 equiv.) was added, followed by the dropwise addition of ethyl vinyl ether (250 g, 3040 mmol) over a period of 45 minutes at −10° C. The reaction mixture was then stirred for 8 h at 0° C. and following this was stirred overnight at RT. The reaction mixture was quenched with saturated NaHCO₃ solution (600 mL) and the organic phase was separated. The aqueous phase was extracted with DCM (2×500 mL). The combined organic phases were washed with water (2×1000 mL), dried over Na₂SO₄ and concentrated under reduced pressure, and the crude product was thereby obtained as a brown oil (450 g).

Step b: Hydrazine dihydrochloride (225 g, 2140 mmol, 1.6 equiv.) was taken up in EtOH (1400 mL) and the mixture was then stirred. TEA (135.4 g (185.4 mL), 1340 mmol, 1 equiv.) was then added dropwise at RT over a period of 45 minutes. The step a product (225 g, crude product) was then added dropwise at RT and the mixture was heated overnight under reflux. EtOH was distilled off and the residue was taken up in iced water (500 mL) and the mixture was then extracted with EA (2×400 mL). The combined extracts were washed with iced water (300 mL), dried over Na₂SO₄ and concentrated under reduced pressure, the crude product being obtained as a white solid (195 g).

Step c: NaH (33.08 g (19.85, 60%), 1.5 equiv.) was added to a small amount of n-hexane and the mixture was stirred for 10 minutes. The n-hexane was decanted, dry DMF (500 mL) was added under an N₂ atmosphere and the mixture was stirred. A solution of the product obtained in step b (75 g, 550 mmol) in DMF (125 mL) was added dropwise under an N₂ atmosphere. A solution of 4-methoxyl benzoyl chloride (86.3 g, 550 mmol, 1 equiv.) in DMF (125 mL) was then added dropwise and the mixture was stirred for 12 h at RT and then poured into iced water (500 mL), and the mixture was extracted with EA (2×400 mL). The mixture was then dried over Na₂SO₄ and concentrated under reduced pressure, the crude product being obtained as a brown oil (125 g, 88% yield).

Step d: Diisopropylamine (28.4 (39.4 mL), 1.2 equiv.) was taken up in THF (500 mL), stirred, and cooled to 0° C. n-BuLi (234.4 mL, 1.5 equiv.) was added dropwise at 0° C. and the mixture was then cooled to −78° C. A solution of the product obtained in step c (62 g, 240 mmol) in THF (200 mL) was added dropwise over a period of 30 minutes and the mixture was stirred for 30 minutes at −78° C. Dry CO₂ gas was then passed through the mixture for 1.5 h. The course of the reaction was followed by thin-layer chromatography (10% EA in n-hexane 15:85, R_{f}˜0.1). After completion of the reaction the mixture was poured into iced water (300 mL) and the aqueous phase was made alkaline and extracted with EA (2×200 mL). The aqueous phase was then acidified with a 20% HCl solution and extracted with EA (2×200 mL). The combined organic phases were dried over Na₂SO₄ and concentrated under reduced pressure. The desired product was obtained as a white solid (42 g, 58% yield).

Step e: DMF was added in a catalytically active amount to a stirred solution of the product obtained from step d (50 g, 160 mol) in DCM (750 mL) and the mixture was cooled to 0° C. Thionyl chloride (99.3 g (61 mL), 830 mmol, 5 equiv.) was added dropwise over 30 minutes at 0° C. The mixture was then slowly heated and refluxed for 2 h. After completion of the reaction the DCM was distilled off. The crude product was taken up in DCM (500 mL) and the resultant solution was added dropwise to an aqueous ammonia solution at 0° C. (600-700 mL). The mixture was then stirred for a further 1 h. Following this iced water was added (200 mL) and the mixture was extracted with EA (2×200 mL). The combined organic phases were dried over Na₂SO₄ and concentrated under reduced pressure. 37 g of the crude product were obtained, which was used without further purification directly in the next step.

Step f: Lithium aluminium hydride (4.7 g, 120 mmol, 1 equiv.) was added to a small amount of n-hexane and the solution was then stirred for 10 minutes. n-hexane was decanted and THF (250 mL) was added to the lithium aluminium hydride. A solution of the product obtained in step e (37 g, 120 mmol) in THF (125 mL) was added dropwise at 0° C. and the mixture was then heated under reflux for 5 h. More lithium aluminium hydride (2,3 g) was then added and the mixture was refluxed for a further 4 h. The mixture was then added to a saturated solution of Na₂SO₄ (1000 mL) and extracted with EA (2×500 mL). The combined organic phases were dried over Na₂SO₄ and concentrated under reduced pressure, 32.5 g of white product being obtained that was used without further purification directly in the next step.

Step g: TEA (22.7 g (30.2 mL), 0.026 mol, 0.8 equiv.) was added dropwise over a period of 10 minutes to a solution of the product obtained in step f ((80 g, 280 mmol) in DCM (600 mL) at 0° C. Di-tert-butyl dicarbonate (61.2 g (62.5 mL), 280 mmol, 1 equiv.) taken up in DCM (200 mL) was then added dropwise over a period of 20-30 minutes at 0° C. The mixture was then stirred for half an hour at 0° C. and half an hour at RT. DCM was distilled off and the residue was taken up in iced water (500 mL) and the product was extracted with EA (2×300 mL). The combined organic phases were dried over Na₂SO₄ and concentrated under reduced pressure to yield the crude product, which was obtained as a white powder (80 g, 74% yield) by recrystallisation from n-hexane (200 mL).

Step h: The step g product (5 g, 12 mmol) was taken up in DCM (30 mL) and cooled to 0° C. HCl gas was passed through the mixture for 45 minutes at 0° C. The DCM was then distilled off and the residue was taken up in iced water (200 mL); the product was extracted with 20% ethyl acetate (EA) in n-hexane (2×100 mL). The aqueous phase was made alkaline to pH˜10 with a 2N NaOH solution and then extracted with EA (5×100 mL). The combined organic phases were washed with water (2×200 ml), dried over Na₂SO₄ and concentrated under reduced pressure, 2.4 g (64% yield) of the product being obtained as a yellow oil.

### 5.5 Preparation of 1-(tetrahydro-2H-pyran-4-yl)-3-(trifluoromethyl)-1H-pyrazol-5-yl)methanamine (for the Synthesis of Example 13)

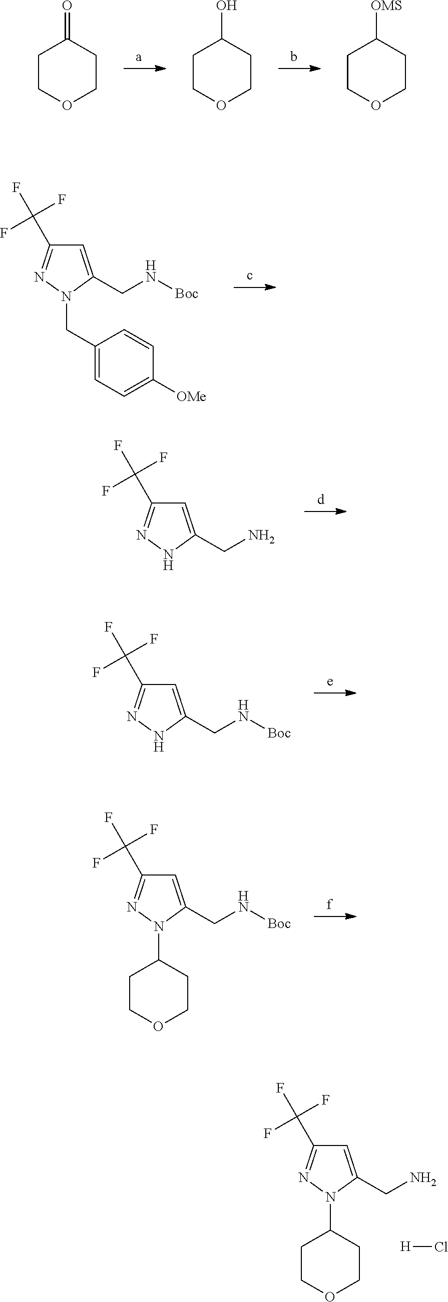

Step a: Tetrahydropyran-4-one (7.5 g, 75 mmol, 1 equiv.) in MeOH (75 mL) was cooled to 0° C. NaBH₄ (1.425 g, 37.5 mmol, 0.5 equiv.) was added in portions at 0° C. The mixture was heated to RT and stirred for 1 h at RT. MeOH was distilled off and the mixture was diluted with iced water, neutralised with acetic acid, and extracted with EA (3×30 mL). The organic phase was concentrated under reduced pressure and the product was obtained as a colourless oil (4.3 g, 56% yield).

Step b: TEA (13 g, 129 mmol, 3 equiv.) was added to the step a product (4.3 g, 43 mmol, 1 equiv.) in DCM (43 mL) and the mixture was cooled to 0° C. Mesyl chloride (4.47 g, 43 mmol, 1 equiv.) was added and the mixture was stirred for 1 h at 0° C. The mixture was then washed with iced water (1×50 mL) and the phases were separated. The organic phase was dried over Na₂SO₄ and concentrated under reduced pressure, 7 g (90% yield) of the product being obtained as a yellow solid.

Step c: AlCl₃ (17.34 g, 129 mmol, 2.5 equiv.) was added in portions over 30 minutes to a stirred solution of tert-butyl (1-(4-methoxybenzyl)-3-(trifluoromethyl)-1H-pyrazol-5-yl)methyl carbamate (20 g, 52 mmol) in Toluene (300 mL). The reaction mixture was heated to 50-60° C. and stirred for 2 h at this temperature. Dilute HCl and iced water (300 mL) were then added and the mixture was extracted with EA (2×100 mL). The aqueous phase was made alkaline with an NaOH solution and then extracted with EA, dried over Na₂SO₄ and concentrated under reduced pressure, a brown crude product thereby being obtained (4.6 g). The crude product was directly used without further purification in the next step.

Step d: The step c product (0.7 g, 42 mmol, 1 equiv.) was taken up in DCM (70 mL) and TEA (5.86 mL, 72 mmol, 1 equiv.) was then added at RT and the mixture was stirred for 10 minutes and then cooled to 0 to −5° C. Di-tert-butyl dicarbonate (9.24 g, 42 mmol, 1 equiv.) was added dropwise over 30 minutes and the mixture was kept at 0 to −5° C. for 3 h. The mixture was then heated to RT and the DCM was distilled of. The residue was taken up in water (50 mL) and extracted with EA (3×100 mL). The combined organic phases were dried over Na₂SO₄ and concentrated under reduced pressure. After column chromatography (SiO₂, EA/n-hexane 1:9, v/v) a white solid was obtained (500 mg, 44% yield).

Step e: NaH (0.54 g, 22 mmol, 2 equiv.) in DMF (10 mL) was cooled to 0° C. The step d product (3 g, 11.3 mmol, 1 equiv.) was added at 0° C. and the solution was kept for 1 h at 0° C. The step b product (3.46 g, 19 mmol, 1.7 equiv.) was added and the mixture was heated to RT and then slowly heated to 90° C. and stirred for 12 h at 90° C. The mixture was then poured into iced water (20 mL) and extracted with EA (3×15 mL). The combined organic phases were dried over Na₂SO₄ and concentrated under reduced pressure. After column chromatography (SiO₂, EA/n-hexane 5:95, v/v) a white solid was obtained (600 mg, 15% yield).

Step f: Step e product (390 mg, 1.1 mmol, 1 equiv.) was taken up in MeOH (3 mL) and HCl in isopropyl alcohol (279 μl, 1.7 mmol, 1.5 equiv.) was added and the mixture was stirred for 16 h at RT. MeOH was distilled off. The residue was taken up in diethyl ether (10 mL) and the mixture was stirred for 10 minutes at RT. The precipitated product was filtered off and washed with diethyl ether, and was obtained as a white solid (133 mg, 42% yield).

### 5.6 Preparation of 3-tert-butyl-1-(3-chlorophenyl)-1H-1,2,4-triazol-5-yl)methanamine dihydrochloride (for the Synthesis of Examples 43 and 44)

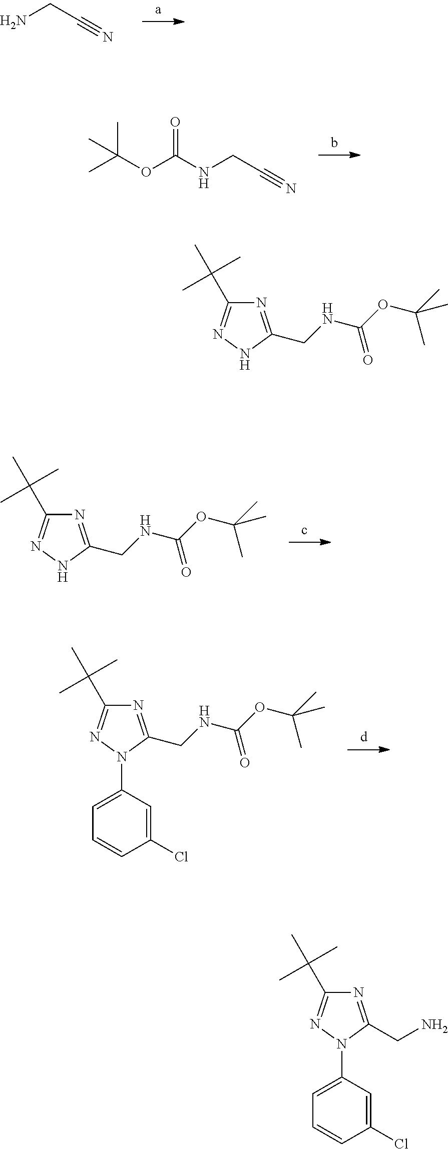

Step a: Aminoacetonitrile hydrochloride (5 g, 54 mmol) was added to dichloromethane (30 mL). A solution of di-tert-butyl dicarbonate (11.9 g, 54.6 mmol, 1.01 equiv.) and TEA (24.6 g, 33.7 mL, 243 mmol, 4.5 equiv.) in dichloromethane (25 mL) was also metered in. After completion of the addition the mixture was heated for 16 h under reflux. After the reaction mixture had cooled it was filtered, the filtrate was washed with water (50 mL), dried over magnesium sulphate and freed from solvent under reduced pressure. N-boc-aminoacetonitrile (5.58 g, 66% yield) remained as a brownish oil, which was used without further purification.

Step b: N-boc-aminoacetonitrile (5.75 g, 36.8 mmol) was added to methanol (90 mL). Sodium methanolate (383 mg, 7.36 mmol, 0.2 equiv.) was added in portions to the solution. The mixture was stirred for 2.5 h at room temperature, pivaloyl hydrazide (4.28 g, 36.8 mmol, 1 equiv.) was added, and the mixture was heated for 18 h under reflux. The solvent was removed under reduced pressure, the residue was taken up in dichloromethane (150 mL), washed with saturated sodium chloride solution (120 mL), the aqueous phase was extracted with dichloromethane (2×50 mL), and the combined organic phases were dried over magnesium sulphate and freed from the solvent under reduced pressure. The residue was purified by column chromatography (SiO₂, methyl-tert-butyl ether/dichloromethane 1/1, v/v). Tert-butyl-(3-tert-butyl-1H-1,2,4-triazol-5-yl)methyl carbamate (5.67 g, 61% yield) was obtained in the form of a colourless solid.

Step c: Copper iodide (28 mg, 0.16 mmol, 0.05 equiv.), potassium carbonate (906 mg, 6.57 mmol, 2.1 equiv.) and tert-butyl-(3-tert-butyl-1H-1,2,4-triazol-5-yl)methyl carbamate (752 mg, 3.13 mmol) were placed in a microwave glass. The glass was evacuated 3 times and flushed with nitrogen. 3-chloro-iodobenzene (893 mg, 3.76 mmol, 1.2 equiv.), N1,N2-dimethylcyclohexane-1,2-diamine (60 mg, 0.47 mmol, 0.15 equiv.) and dimethylformamide (8 mL) were added under a stream of nitrogen. The reaction vessel was sealed in an airtight manner and stirred for 24 h at 110° C. The reaction mixture was cooled to room temperature, the solvent was removed under reduced pressure, and the residue was taken up in EtOAc (70 mL) and extracted with saturated sodium hydrogen carbonate solution (70 mL). The aqueous phase was extracted with EtOAc (2×10 mL), and the combined organic phases were dried over magnesium sulphate and concentrated by evaporation. The residue was purified by column chromatography (SiO₂, EtOAc/n-hexane 1/4, v/v). Tert-butyl-(3-tert-butyl-1-(3-chlorophenyl)-1H-1,2,4-triazol-5-yl)methyl carbamate (773 mg, 68% yield) was obtained in the form of a colourless solid.

Step d: Tert-butyl-(3-tert-butyl-1-(3-chlorophenyl)-1H-1,2,4-triazol-5-yl)methyl carbamate (753 mg, 2.06 mmol) was placed in a reaction flask. HCl in 1,4-dioxane (3.3 mL, c=4 mol/L, 13.2 mmol, 6.4 equiv.) and 1,4-dioxane (14 mL) were also added. The mixture was stirred for 60 h at room temperature. A yellowish white suspension was formed. The precipitate was suction filtered, washed with 1,4-dioxane (2×5 mL) and dried. (3-tert-butyl-1-(3-chlorophenyl)-1H-1,2,4-triazol-5-yl)methanamine dihydrochloride (753 mg, quantitative) was obtained in the form of a colourless solid.

### 5.7 Preparation of (1-hexyl-3-(trifluoromethyl)-1H-1,2,4-triazol-5-yl)methanamine dihydrochloride (for the Synthesis of Example 45)

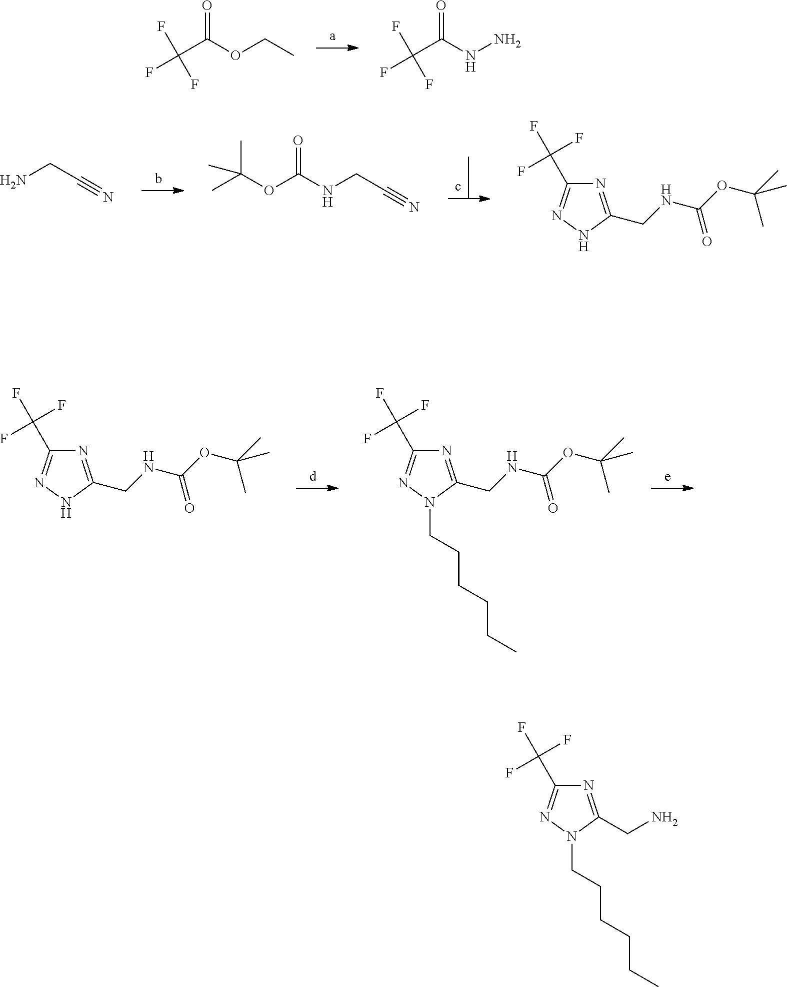

Step a: Ethyl trifluoroacetate (1.03 g, 0.865 mL, 7.25 mmol) and hydrazine monohydrate (80% w/w, 0.498 g, 0.475 mL, 7.97 mmol, 1.1 equiv.) were dissolved in ethanol (1.4 mL) and heated under reflux for 3 h. The solvent was removed under reduced pressure, and the residue was taken up in EtOAc (10 mL) and extracted with water (10 mL). The aqueous phase was extracted with EtOAc (4×10 mL), the combined organic phases were washed with water (5 mL), dried over magnesium sulphate and freed from the solvent under reduced pressure. The residue was purified by column chromatography (SiO₂, EtOAc/n-hexane 1/2, v/v). 504 mg of trifluoroacetic acid hydrazide (3.94 mmol, 54% yield) were obtained in the form of a colourless oil.

Step b: Aminoacetonitrile hydrochloride (5.00 g, 54.0 mmol) was added to 30 mL of dichloromethane. A solution of di-tert.-butyl dicarbonate (11.9 g, 54.6 mmol, 1.01 Aq.) and TEA (24.6 g, 33.7 mL, 243 mmol, 4.5 equiv.) in dichloromethane (25 mL) was additionally metered in. After completion of the addition the mixture was heated under reflux for 16 h. After the reaction mixture had cooled it was filtered, the filtrate was washed with water (50 mL), dried over magnesium sulphate and freed from solvent under reduced pressure. N-boc-aminoacetonitrile (5.58 g, 66% yield) remained as a brownish oil, which was used without further purification.

Step c: N-boc-aminoacetonitrile (3.81 g, 24.4 mmol) was added to methanol (75 mL). Sodium methanolate (254 mg, 4.88 mmol, 0.2 equiv.) was added in portions to the solution. The mixture was stirred for 2.5 h at room temperature, trifluoroacetic acid hydrazide (3.12 g, 24.4 mmol, 1 equiv.) dissolved in MeOH (10 mL) was added, and the mixture was heated under reflux for 18 h. The solvent was removed under reduced pressure, the residue was taken up in dichloromethane (200 mL), washed with saturated sodium chloride solution (120 mL), the aqueous phase was extracted with dichloromethane (2×50 mL), and the combined organic phases were dried over magnesium sulphate and freed from the solvent under reduced pressure. The residue was purified by column chromatography (SiO₂, methyl-tert-butyl ether/dichloromethane 1/2, v/v). Tert-butyl-(3-(trifluoromethyl)-1H-1,2,4-triazol-5-yl)methyl carbamate (3.84 g, 59% yield) was obtained in the form of a colourless solid.

Step d: Tert-butyl-(3-(trifluoromethyl)-1H-1,2,4-triazol-5-yl)methyl carbamate (101 mg, 0.379 mmol) was added at 0° C. to a suspension of sodium hydride (60% w/w in mineral oil, 18 mg, 0.47 mmol, 1.25 Aq.) in dimethylformamide (1.2 mL). The mixture was stirred for 45 min at 0° C., heated to room temperature, and n-hexyl iodide (301 mg, 1.42 mmol, 3.75 equiv.) was added. The mixture was stirred for 18 h at room temperature, water (10 mL) was added, and the mixture was extracted with EtOAc (6×15 mL). The combined organic phases were washed with water (5 mL) and saturated sodium chloride solution (10 mL), dried over magnesium sulphate and freed from the solvent under reduced pressure. The residue was purified by column chromatography (SiO₂, EtOAc/n-hexane 1/9, v/v). Tert-butyl-(1-hexyl-3-(trifluoromethyl)-1H-1,2,4-triazol-5-yl)methyl carbamate was obtained (141 mg, 86% yield).

Step e: Tert-butyl-(1-hexyl-3-(trifluoromethyl)-1H-1,2,4-triazol-5-yl)methyl carbamate (549 mg, 1.57 mmol) was placed in a reaction flask. HCl in 1,4-dioxane (2.5 mL, c=4 mol/L, 10 mmol, 6.4 equiv.) and 1,4-dioxane (10 mL) were also added. The mixture was stirred for 60 h at room temperature. The reaction mixture was freed from the solvent under reduced pressure. (1-hexyl-3-(trifluoromethyl)-1H-1,2,4-triazol-5-yl)methanamine dihydrochloride (465 mg, 92% yield) was obtained in the form of a colourless solid.

### Synthesis of the Example Compounds

### 1. Preparation of Amides (A=CH or C)

General instructions for converting amines of the general formula (II) with carboxylic acids of the general formula (III) or carboxylic acid derivates of the general formula (IV) to compounds of the general formula (I) where A=CH or C (amides) according to scheme 1a (step j09).

### 1.1 Method A:

The acid of the general formula (III) (1 equivalent), the amine of the general formula (II) (1.2 equivalents) and EDCI (1.2 equivalents) are stirred in DMF (10 mmol acid/20 mL) for 12 hours at RT and water is then added. The reaction mixture is extracted several times with EE, the aqueous phase is saturated with NaCl and then re-extracted with EE. The combined organic phases are washed with 1HCl and brine, dried over magnesium sulphate, and the solvent is removed in vacuo. The residue is purified by means of flash chromatography (SiO₂, EE/hexane in various mixing ratios such as e.g. 1:2) and the product (I) is thereby obtained.

### 1.2 Method B:

The acid of the general formula (III) (1 equivalent) and the amine of the general formula (II) (1.1 equivalents) are dissolved in dichloromethane (1 mmol acid in 6 mL), and EDCI (1.5 equivalent), HOBt (1.4 equivalents) and triethylamine (3 equivalents) are added at 0° C. The reaction mixture is stirred 20 h at room temperature and the crude product is purified by means of column chromatography (SiO₂, n-hexane/EE in various mixing ratios such as e.g. 2:1) and (I) is thereby obtained.

### 1.3 Method C:

A chlorinating agent, preferably thionyl chloride, is first of all added to the acid of the general formula (III) (1 equivalent) and the mixture thereby obtained is boiled under reflux and the acid (III) is thereby converted into the corresponding acid chloride (IV). The amine of the general formulae (II) (1.1 equivalents) dissolved in dichloromethane (1 mmol acid in 6 mL) triethylamine (3 equivalents) is added at 0° C. The reaction mixture is stirred for 20 h at room temperature and the crude product is purified by means of column chromatography (SiO₂, n-hexane/EE in various mixing ratios such as e.g. 2:1) and (I) is thereby obtained.

### 1.4 Method D:

The phenyl ester (IVa) (1 equivalent) and the amine (II) (1,1 equivalents) are dissolved in THF (10 mmol of the reaction mixture in 120 ml) and stirred for 16 h at room temperature after adding DBU (1.5 equivalent). After distilling of the solvent under reduced pressure the crude product is purified by means of column chromatography (SiO₂, n-hexane/EE in various mixing ratios such as e.g. 2:1) and (I) is thereby obtained.

The following example compounds 30-33, 35-38 and 42 can be obtained according to one of the methods described hereinbefore.

| | |
|---|---|
| 30 | N-((1-(3-chlorophenyl)-3-(trifluoromethyl)-1H-pyrazol-5-yl)methyl)- |
| | 1-methylpiperidine-4-carboxamide |
| 31 | 1-acetyl-N-((1-(3-chlorophenyl)-3-(trifluoromethyl)-1H-pyrazol-5- |
| | yl)methyl)piperidine-4-carboxamide |
| 32 | 1-benzoyl-N-((3-tert-butyl-1-(3-chlorophenyl)-1H-pyrazol-5- |
| | yl)methyl)piperidine-4-carboxamide |
| 33 | N-((1-(3-chlorophenyl)-3-(trifluoromethyl)-1H-pyrazol-5-yl)methyl)- |
| | 4-isopropylcyclohexane carboxamide |
| 35 | N-((1-(3-chlorophenyl)-3-(trifluoromethyl)-1H-pyrazol-5-yl)methyl)- |
| | 4-hydroxycyclohex-1-ene carboxamide |
| 36 | N-((1-(3-chlorophenyl)-3-(trifluoromethyl)-1H-pyrazol-5-yl)methyl)- |
| | 1-ethyl-1,2,3,6-tetrahydropyridine-4-carboxamide |
| 37 | N-((1-(3-chlorophenyl)-3-(trifluoromethyl)-1H-pyrazol-5-yl)methyl)- |
| | 1-(4-fluorophenylsulfonyl)-1,2,3,6-tetrahydropyridine-4-carboxamide |
| 38 | N-((1-(3-chlorophenyl)-3-(trifluoromethyl)-1H-pyrazol-5-yl)methyl)- |
| | 4-ethylcyclohex-3-ene carboxamide |
| 42 | N-((1-(3-chlorophenyl)-3-(trifluoromethyl)-1H-pyrazol-5-yl)methyl)- |
| | 1-(3-chloropyridine-2-yl)-1,2,3,6-tetrahydropyridine-4-carboxamide |

### 2. Preparation of Ureas (A=N)

General procedure for converting amines of the general formula (II) or (VI) with phenyl chloroformate to form compounds of the formula (V) or (Via) (step j07 and v1) and subsequent conversions with amines of the general formula (VI) or (II) to form compounds of the general formula (I) where A=N according to scheme 1a or 1c (step j08 and v2):

Step j07/v1: The amine of the general formula (II) or (VI) (1 equivalent) is added to dichloromethane (10 mmol amine in 70 mL) and phenyl chloroformate (1.1 equivalent) is added at room temperature and the mixture is stirred for 30 min. After removing the solvent in vacuo the residue is purified by means of flash chromatography (SiO₂, solvent mixtures of diethyl ether/hexane in ratios such as 1:2) and (V) or (Via) is thereby obtained.

Step j08/v2: The obtained phenyl carbamate (V) or (Via) (1 equivalent) and the corresponding amine (VI) or (II) (1.1 equivalent) are dissolved in THF (10 mmol of the reaction mixture in 120 mL) and after adding DBU (1.5 equivalent) the reaction mixture is stirred for 16 h at room temperature. After removing solvent in vacuo the resultant residue is purified by means of flash chromatography (SiO₂, solvent mixtures of diethyl ether/hexane in ratios such as 1:1) and (I) is thereby obtained.

The following example compounds 1-3, 5, 9, 10, 22, 24-26, 34 as well as 46-56 were obtained according to one of the methods described hereinbefore. The following example compounds 4, 6-8, 11-21, 23, 27-33 and 35-45 can be obtained by one of the methods described hereinbefore.

| | |
|---|---|
| 1 | N-((3-tert-butyl-1-(3-chlorophenyl)-1H-pyrazol-5-yl)methyl)-4-(3- |
| | (trifluoromethyl)pyridine-2-yl)piperazine-1-carboxamide |
| 2 | N-((3-tert-butyl-1-(3-chlorophenyl)-1H-pyrazol-5-yl)methyl)-4-(3-chloropyridine-2- |
| | yl)piperazine-1-carboxamide |
| 3 | N-((1-(3-chlorophenyl)-3-(trifluoromethyl)-1H-pyrazol-5-yl)methyl)-4-(3- |
| | chloropyridine-2-yl)piperazine-1-carboxamide |
| 4 | N-((1-(4-chlorophenyl)-3-(trifluoromethyl)-1H-pyrazol-5-yl)methyl)-4-(3- |
| | chloropyridine-2-yl)piperazine-1-carboxamide |
| 5 | N-((3-tert-butyl-1-(3-chlorophenyl)-1H-pyrazol-5-yl)methyl)-4-(2- |
| | fluorophenyl)piperazine-1-carboxamide |
| 6 | N-((3-tert-butyl-1-(3-chloro-4-fluorophenyl)-1H-pyrazol-5-yl)methyl)-4-(3- |
| | chloropyridine-2-yl)piperazine-1-carboxamide |
| 7 | N-((3-tert-butyl-1-(pyridin-2-yl)-1H-pyrazol-5-yl)methyl)-4-(3-chloropyridine-2- |
| | yl)piperazine-1-carboxamide |
| 8 | 4-(3-chloropyridin-2-yl)-N-((1-m-tolyl-3-(trifluoromethyl)-1H-pyrazol-5- |
| | yl)methyl)piperazine-1-carboxamide |
| 9 | N-((1-(3-chlorophenyl)-4-methyl-3-(trifluoromethyl)-1H-pyrazol-5-yl)methyl)-4-(3- |
| | chloropyridin-2-yl)piperazine-1-carboxamide |
| 10 | N-((1-(3-chlorophenyl)-3-cyclopropyl-1H-pyrazol-5-yl)methyl)-4-(3-chloropyridin- |
| | 2-yl)piperazine-1-carboxamide |
| 11 | 4-(3-chloropyridin-2-yl)-N-((1-(4-methoxybenzyl)-3-(trifluoromethyl)-1H-pyrazol-5- |
| | yl)methyl)piperazine-1-carboxamide |
| 12 | 4-(3-chloropyridin-2-yl)-N-((1-pentyl-3-(trifluoromethyl)-1H-pyrazol-5- |
| | yl)methyl)piperazine-1-carboxamide |
| 13 | 4-(3-chloropyridin-2-yl)-N-((1-(tetrahydro-2H-pyran-4-yl)-3-(trifluoromethyl)-1H- |
| | pyrazol-5-yl)methyl)piperazine-1-carboxamide |
| 14 | N-((1-(3-chlorophenyl)-3-(trifluoromethyl)-1H-pyrazol-5-yl)methyl)-4- |
| | methylpiperazine-1-carboxamide |
| 15 | N-((1-(3-chlorophenyl)-3-(trifluoromethyl)-1H-pyrazol-5-yl)methyl)-4- |
| | ethylpiperazine-1-carboxamide |
| 16 | 4-tert-butyl-N-((1-(3-chlorophenyl)-3-(trifluoromethyl)-1H-pyrazol-5- |
| | yl)methyl)piperazine-1-carboxamide |
| 17 | N-((1-(3-chlorophenyl)-3-(trifluoromethyl)-1H-pyrazol-5-yl)methyl)-4- |
| | cyclohexylpiperazine-1-carboxamide |
| 18 | N-((1-(3-chlorophenyl)-3-(trifluoromethyl)-1H-pyrazol-5-yl)methyl)-4-(thiophen-2- |
| | yl)piperazine-1-carboxamide |
| 19 | N-((1-(3-chlorophenyl)-3-(trifluoromethyl)-1H-pyrazol-5-yl)methyl)-4- |
| | phenylpiperazine-1-carboxamide |
| 20 | 4-benzyl-N-((3-tert-butyl-1-(3-chlorophenyl)-1H-pyrazol-5-yl)methyl)piperazine-1- |
| | carboxamide |
| 21 | N-((3-tert-butyl-1-(3-chlorophenyl)-1H-pyrazol-5-yl)methyl)-4-(1- |
| | phenylethyl)piperazine-1-carboxamide |
| 22 | N-((1-(3-chlorophenyl)-3-(trifluoromethyl)-1H-pyrazol-5-yl)methyl)-4-(1-(4- |
| | fluorophenyl)ethyl)piperazine-1-carboxamide |
| 23 | N-((1-(3-chlorophenyl)-3-(trifluoromethyl)-1H-pyrazol-5-yl)methyl)-4- |
| | (methylsulfonyl)piperazine-1-carboxamide |
| 24 | 4-acetyl-N-((1-(3-chlorophenyl)-3-(trifluoromethyl)-1H-pyrazol-5- |
| | yl)methyl)piperazine-1-carboxamide |
| 25 | 4-benzoyl-N-((1-(3-chlorophenyl)-3-(trifluoromethyl)-1H-pyrazol-5- |
| | yl)methyl)piperazine-1-carboxamide |
| 26 | N-((1-(3-chlorophenyl)-3-(trifluoromethyl)-1H-pyrazol-5-yl)methyl)-4- |
| | phenylpiperidine-1-carboxamide |
| 27 | N-((1-(3-chlorophenyl)-3-(trifluoromethyl)-1H-pyrazol-5-yl)methyl)-4-(2- |
| | methoxyphenyl)piperidine-1-carboxamide |
| 28 | N-((1-(3-chlorophenyl)-3-(trifluoromethyl)-1H-pyrazol-5-yl)methyl)-4-(2,4- |
| | difluorophenyl)piperidine-1-carboxamide |
| 29 | N-((1-(3-chlorophenyl)-3-(trifluoromethyl)-1H-pyrazol-5-yl)methyl)-4-hydroxy-4- |
| | phenylpiperidine-1-carboxamide |
| 34 | N-((1-(3-chlorophenyl)-3-(trifluoromethyl)-1H-pyrazol-5-yl)methyl)-4-(4- |
| | fluorophenyl)-5,6-dihydropyridine-1(2H)-carboxamide |
| 39 | (S)-4-(3-chloro-5-(1,2-dihydroxyethyl)pyridin-2-yl)-N-((1-(3-chlorophenyl)-3- |
| | (trifluoromethyl)-1H-pyrazol-5-yl)methyl)piperazine-1-carboxamide |
| 40 | (S)-4-(3-chlor-5-(1,2-dihydroxyethyl)pyridin-2-yl)-N-((1-(3-chlorophenyl)-3- |
| | (trifluoromethyl)-1H-pyrazol-5-yl)methyl)-5,6-dihydropyridine-1(2H)-carboxamide |
| 41 | (S)-4-(3-chlor-5-(1,2-dihydroxyethyl)pyridin-2-yl)-N-((1-(3-chlorophenyl)-3- |
| | (trifluoromethyl)-1H-pyrazol-5-yl)methyl)-4-fluoropiperidine-1-carboxamide |
| 43 | N-((3-tert-butyl-1-(3-chlorophenyl)-1H-1,2,4-triazol-5-yl)methyl)-4-(3- |
| | chloropyridin-2-yl)piperazine-1-carboxamide |
| 44 | N-((3-tert-butyl-1-(3-chlorophenyl)-1H-1,2,4-triazol-5-yl)methyl)-4-(1-(4- |
| | fluorophenyl)ethyl)piperazine-1-carboxamide |
| 45 | 4-(1-(4-fluorophenyl)ethyl)-N-((1-hexyl-3-(trifluoromethyl)-1H-1,2,4-triazol-5- |
| | yl)methyl)piperazine-1-carboxamide |
| 46 | N-((1-(3-chlorophenyl)-3-(trifluoromethyl)-1H-pyrazol-5-yl)methyl)-4-(2- |
| | fluorophenyl)piperazine-1-carboxamide |
| 47 | N-((1-(3-chlorophenyl)-3-(trifluoromethyl)-1H-pyrazol-5-yl)methyl)-4-(4- |
| | fluorophenyl)piperazine-1-carboxamide |
| 48 | N-((1-(3-chlorophenyl)-3-(trifluoromethyl)-1H-pyrazol-5-yl)methyl)-4-(2- |
| | methoxyphenyl)piperazine-1-carboxamide |
| 49 | N-((1-(3-chlorophenyl)-3-(trifluoromethyl)-1H-pyrazol-5-yl)methyl)-4-(3- |
| | methoxyphenyl)piperazine-1-carboxamide |
| 50 | N-((1-(3-chlorophenyl)-3-(trifluoromethyl)-1H-pyrazol-5-yl)methyl)-4-(4- |
| | methoxyphenyl)piperazine-1-carboxamide |
| 51 | 4-(2-chlorophenyl)-N-((1-(3-chlorophenyl)-3-(trifluoromethyl)-1H-pyrazol-5- |
| | yl)methyl)piperazine-1-carboxamide |
| 52 | N-((3-tert-butyl-1-(3-chlorophenyl)-1H-pyrazol-5-yl)methyl)-4-(3- |
| | chlorophenyl)piperazine-1-carboxamide |
| 53 | N-((3-tert-butyl-1-(3-chlorophenyl)-1H-pyrazol-5-yl)methyl)-4-(4- |
| | chlorophenyl)piperazine-1-carboxamide |
| 54 | 4-(4-chlorophenyl)-N-((1-(3-chlorophenyl)-3-(trifluoromethyl)-1H-pyrazol-5- |
| | yl)methyl)piperazine-1-carboxamide |
| 55 | N-((3-tert-butyl-1-(3-chlorophenyl)-1H-pyrazol-5-yl)methyl)-4-(3- |
| | (trifluoromethyl)pyridine-2-yl)piperazine-1-carboxamide |
| 56 | N-((1-(3-chlorophenyl)-3-(trifluoromethyl)-1H-pyrazol-5-yl)methyl)-4-(3- |
| | (trifluoromethyl)pyridin-2-yl)piperazine-1-carboxamide |

For some of the example compounds the experimentally obtained mass spectrometry data are given hereinafter by way of example:

| Example | [M + H] |
|---|---|
| 1 | 521.1 |
| 2 | 487.2 |
| 3 | 499.4 |
| 5 | 469.9 |
| 22 | 498.1 |

### Pharmacological Data

The affinity of the compounds according to the invention for the vanilloid receptor 1 (VR1/TRPV1 receptor) was determined as described hereinbefore (Pharmacological Methods I and II). The compounds according to the invention of the formula (I) shown hereinbefore have an excellent affinity for the VR1/TRPV1 receptor (Table 1.).

In Table 1 the following abbreviations have the following meanings:

### Cap=Capsaicin

FTm=result of the formalin test
p.o.=oral

The value after the symbol “@” gives the concentration at which the inhibition (in percent) was determined in each case.

TABLE 1

| Compound | | FTm, p.o. % |
|---|---|---|
| according | Kᵢ (human) | effect @ |
| to example | [nM] Cap | dose (mg/kg) |
| 1 | 3 | |
| 2 | 10 | 1% @10.0 |
| | | 36% @30.0 |
| | | 66% @100.0 |
| 3 | 14 | |
| 5 | 5 | |
| 9 | 16 | |
| 10 | 85 | |
| 12 | 68 | |
| 22 | 41 | |
| 26 | 27 | |
| 34 | 27 | |
| 46 | 45 | |
| 47 | 48 | |
| 48 | 61 | |
| 49 | 35 | |
| 50 | 63 | |
| 51 | 7 | |
| 52 | 36 | |
| 53 | 42 | |
| 54 | 56 | |
| 55 | 3 | |
| 56 | 4 | |

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A compound corresponding to the formula (I): 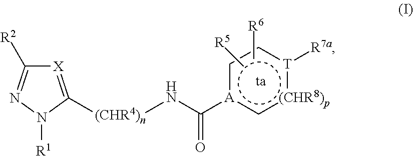
wherein
X denotes CR³ or N, wherein
R³ denotes H; or denotes C₁₋₁₀-alkyl, saturated or unsaturated, branched or unbranched, unsubstituted or monosubstituted or polysubstituted;
A denotes N, C or CH;
T denotes N, C or CR^{7b},
the symbol [symbol] denotes that the non-aromatic ring to can optionally include at least one unsaturated bond,
with the proviso that when A denotes N, A is not part of an unsaturated bond, and
with the proviso that when T denotes N, T is not part of an unsaturated bond,
p denotes 1, 2 or 3;
n denotes 1, 2, 3 or 4;
R⁰ denotes C₁₋₁₀-alkyl, saturated or unsaturated, branched or unbranched, unsubstituted or monosubstituted or polysubstituted; C₃₋₁₀-cycloalkyl or heterocyclyl, in each case saturated or unsaturated, unsubstituted or monosubstituted or polysubstituted; aryl or heteroaryl, in each case unsubstituted or monosubstituted or polysubstituted; C₃₋₁₀-cycloalkyl or heterocyclyl bridged via C₁₋₈-alkyl, in each case saturated or unsaturated, unsubstituted or monosubstituted or polysubstituted, wherein the alkyl chain can in each case be branched or unbranched, saturated, or unsaturated, unsubstituted, monosubstituted or polysubstituted; or aryl or heteroaryl bridged via C₁₋₈-alkyl, in each case unsubstituted or monosubstituted or polysubstituted, wherein the alkyl chain can in each case be branched or unbranched, saturated or unsaturated, unsubstituted, monosubstituted or polysubstituted;
R¹ denotes H; C₁₋₁₀-alkyl, saturated or unsaturated, branched or unbranched, unsubstituted or monosubstituted or polysubstituted; C₃₋₁₀-cycloalkyl¹ or heterocyclyl¹, in each case saturated or unsaturated, unsubstituted or monosubstituted or polysubstituted; aryl or heteroaryl, in each case unsubstituted or monosubstituted or polysubstituted; C₃₋₁₀-cycloalkyl¹ or heterocyclyl¹ bridged via C₁₋₈-alkyl, in each case saturated or unsaturated, unsubstituted or monosubstituted or polysubstituted, wherein the alkyl chain can in each case be branched or unbranched, saturated or unsaturated, unsubstituted, monosubstituted or polysubstituted; or aryl or heteroaryl bridged via C₁₋₈-alkyl, in each case unsubstituted or monosubstituted or polysubstituted, wherein the alkyl chain can in each case be branched or unbranched, saturated or unsaturated, unsubstituted, monosubstituted or polysubstituted; C(═O)—R⁰; C(═O)—OH; C(═O)—OR⁰; C(═O)—NHR⁰; C(═O)—N(R⁰)₂; OH; O—R⁰; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; N(R⁰)(S(═O)₂—R⁰; or SCl₃;
R² denotes H; R⁰; NO₂; CN; OH; SH; F; Cl; Br; I; CF₃; CF₂H; CFH₂; CF₂Cl; CFCl₂; CH₂CF₃; OCF₃; OCF₂H; OCFH₂; OCF₂Cl; OCFCl₂; SCF₃; SCF₂H; SCFH₂; SCF₂Cl; SCFCl₂; S(═O)₂—CF₃; S(═O)₂—CF₂H; S(═O)₂—CFH₂; or SF₅;
R⁴ denotes H;
R⁵, R⁶ and R⁸ each independently denote H; F; Cl; Br; I; OH; OR⁰; or R⁰;
R^{7a} denotes R⁰; C(═O)—R⁰; C(═O)OH; C(═O)—OR⁰; C(═O)—NHR⁰; C(═O)—N(R⁰)₂; OH; O—R^{7c}; SH; S—R⁰; S(═O)₂—R⁰; S(═O)₂—OR⁰; S(═O)₂—NHR⁰; S(═O)₂—N(R⁰)₂; NH₂; NHR⁰; N(R⁰)₂; NH—S(═O)₂—R⁰; or))N(R⁰)(S(═O)₂—R⁰);
R^{7b} denotes H; F; Cl; Br; I; or OH;
with the proviso that R^{7a} cannot denote OH if T denotes CR^{7b} and R^{7b} denotes OH;
R^{7c} denotes C₁₋₁₀-alkyl, saturated or unsaturated, branched or unbranched, unsubstituted or monosubstituted or polysubstituted; or denotes aryl or heteroaryl;
with the proviso that R^{7a} cannot denote NH₂; NHR⁰; N(R⁰)₂; NH—S(═O)₂—R⁰; N(R⁰)(S(═O)₂—R⁰) if T denotes N;
wherein “alkyl substituted”, “heterocyclyl substituted” and “cycloalkyl substituted” in the corresponding radicals denotes the replacement of one or more hydrogen atoms, in each case independently of one another, by F; Cl; Br; I; NO₂; CN; ═O; ═NH; ═C(NH₂)₂; OF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—O(═O)—O—R⁰; NR⁰—O(═O)—NH₂; NR⁰—O(═O)—NH—R⁰; NR⁰—O(═O)—N(R)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or)S(═O)₂N(R⁰)₂;
wherein “cycloalkyl¹ substituted” and “heterocyclyl¹ substituted” in the corresponding radicals denotes the replacement of one or more hydrogen atoms, in each case independently of one another, by F; Cl; Br; I; NO₂; CN; ═O; ═C(NH₂)₂; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R⁰)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
wherein “aryl substituted” and “heteroaryl substituted” in the corresponding radicals denotes the substitution of one or more hydrogen atoms, in each case independently of one another, by F; Cl; Br; I; NO₂; CN; CF₃; CF₂H; CFH₂; CF₂Cl; CFCl₂; R⁰; C(═O)H; C(═O)R⁰; CO₂H; C(═O)OR⁰; CONH₂; C(═O)NHR⁰; C(═O)N(R⁰)₂; OH; OCF₃; OCF₂H; OCFH₂; OCF₂Cl; OCFCl₂; OR⁰; O—C(═O)—R⁰; O—C(═O)—O—R⁰; O—(C═O)—NH—R⁰; O—C(═O)—N(R)₂; O—S(═O)₂—R⁰; O—S(═O)₂OH; O—S(═O)₂OR⁰; O—S(═O)₂NH₂; O—S(═O)₂NHR⁰; O—S(═O)₂N(R⁰)₂; NH₂; NH—R⁰; N(R⁰)₂; NH—C(═O)—R⁰; NH—C(═O)—O—R⁰; NH—C(═O)—NH₂; NH—C(═O)—NH—R⁰; NH—C(═O)—N(R⁰)₂; NR⁰—C(═O)—R⁰; NR⁰—C(═O)—O—R⁰; NR⁰—C(═O)—NH₂; NR⁰—C(═O)—NH—R⁰; NR⁰—C(═O)—N(R⁰)₂; NH—S(═O)₂OH; NH—S(═O)₂R⁰; NH—S(═O)₂OR⁰; NH—S(═O)₂NH₂; NH—S(═O)₂NHR⁰; NH—S(═O)₂N(R⁰)₂; NR⁰—S(═O)₂OH; NR⁰—S(═O)₂R⁰; NR⁰—S(═O)₂OR⁰; NR⁰—S(═O)₂NH₂; NR⁰—S(═O)₂NHR⁰; NR⁰—S(═O)₂N(R⁰)₂; SH; SCF₃; SCF₂H; SCFH₂; SCF₂Cl; SCFCl₂; SR⁰; S(═O)R⁰; S(═O)₂R⁰; S(═O)₂OH; S(═O)₂OR⁰; S(═O)₂NH₂; S(═O)₂NHR⁰; or S(═O)₂N(R)₂;
in the form of a free compound; a tautomer; an N-oxide; a racemate; an isolated enantiomer or diastereomer, a mixture of enantiomers or diastereomers; or in the form of a salt of a physiologically compatible acid or base.

2. A compound according to claim 1, wherein:
X denotes CR³ or N, wherein
R³ denotes H; or denotes unsubstituted C₁₋₁₀-alkyl, saturated or unsaturated, branched or unbranched; and
P denotes 1.

3. A compound according to claim 1, wherein:
R¹ denotes the partial structure (T1) 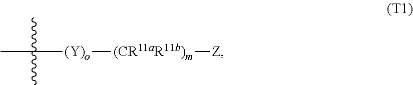
wherein
Y denotes C(═O), O, S, S(═O)₂ or NR¹², wherein
R¹² denotes H; C₁₋₈-alkyl or S(═O)₂—C₁₋₈-alkyl, in which C₁₋₈-alkyl can in each case be saturated or unsaturated, branched or unbranched, unsubstituted or monosubstituted or polysubstituted with one or more substituents independently selected from the group consisting of F, Cl, Br, I, OH, O—C₁₋₄-alkyl, OCF₃, NH₂, NH—C₁₋₄-alkyl and N(C₁₋₄-alkyl)₂;
o denotes 0 or 1;
R^{11a} and R^{11b} each independently denote H; F; Cl; Br; I; NO₂; CF₃; CN; OH; OCF₃; NH₂; C₁₋₄-alkyl, O—C₁₋₄-alkyl, NH—C₁₋₄-alkyl, or N(C₁₋₄-alkyl)₂, wherein C₁₋₄-alkyl can in each case be saturated or unsaturated, branched or unbranched, unsubstituted or monosubstituted or polysubstituted with one or more substituents independently selected from the group consisting of F, Cl, Br, I, O—C₁₋₄-alkyl, OH and OCF₃;
with the proviso that if R^{11a} and R^{11b} are bonded to the same carbon atom, only one of the substituents R^{11a} and R^{11b} can denote OH, OCF₃, NH₂, O—C₁₋₄-alkyl, NH—C₁₋₄-alkyl or N(C₁₋₄-alkyl)₂;
m denotes 0, 1, 2, 3 or 4; and
Z denotes C₁₋₄-alkyl, saturated or unsaturated, branched or unbranched, unsubstituted or monosubstituted or polysubstituted with one or more substituents each independently selected from the group consisting of F, Cl, Br, I, NO₂, CN, OH, ═O, O—C₁₋₄-alkyl, OCF₃, C(═O)—OH, CF₃, NH₂, NH(C₁₋₄-Alkyl), N(C₁₋₄-alkyl)₂, SH, S—C₁₋₄-alkyl, SCF₃ and S(═O)₂OH; C₃₋₁₀-cycloalkyl¹ or heterocyclyl¹, in each case saturated or unsaturated, unsubstituted or monosubstituted or polysubstituted with one or more substituents each independently selected from the group consisting of F, Cl, Br, I, NO₂, CN, OH, O—C₁₋₄-alkyl, OCF₃, C₁₋₄-alkyl, C(═O)—OH, CF₃, SH, S—C₁₋₄-alkyl, SCF₃, S(═O)₂OH, benzyl, phenyl, pyridyl and thienyl, wherein benzyl, phenyl, pyridyl, thienyl can in each case be unsubstituted or monosubstituted or polysubstituted with one or more substituents independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; aryl or heteroaryl, in each case unsubstituted or monosubstituted or polysubstituted with one or more substituents each independently selected from the group consisting of F, Cl, Br, I, NO₂, CN, OH, O—C₁₋₄-alkyl, OCF₃, C₁₋₄-alkyl, C₁₋₄-alkyl monosubstituted or disubstituted with OH, C(═O)—OH, OF₃, NH₂, NH(C₁₋₄-alkyl), N(C₁₋₄-alkyl)₂, SH, S—C₁₋₄-alkyl, SOF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents independently selected from the group consisting of F, Cl, Br, I, NO₂, CN, OH, O—C₁₋₈-alkyl, OCF₃, C₁₋₄-alkyl, C(═O)—OH, OF₃, NH₂, NH(C₁₋₄-alkyl), N(C₁₋₄-Alkyl)₂, SH, S—C₁₋₄-alkyl, SCF₃ and S(═O)₂OH.

4. A compound according to claim 3, wherein:
Y denotes C(═O), O, S, S(═O)₂ or NR¹², wherein
R¹² denotes H; methyl; ethyl; n-propyl; iso-propyl; n-butyl; sec.-butyl; tert.-butyl; S(═O)₂-methyl or S(═O)₂-ethyl;
o denotes 0 or 1;
R^{11a} and R^{11b} each independently denote H; F; Cl; Br; I; NO₂; OF₃; CN; methyl; ethyl; n-propyl; iso-propyl; n-butyl; sec.-butyl; tert.-butyl; CH₂CF₃; OH; O-methyl; O-ethyl; O—(CH₂)₂—O—CH₃; O—(CH₂)₂—OH; OCF₃; NH₂; NH-methyl; N(methyl)₂; NH-ethyl; N(ethyl)₂; or N(methyl)(ethyl);
with the proviso that if R^{11a} and R^{11b} are bonded to the same carbon atom, only one of the substituents R^{11a} and R^{11b} can denote OH; OCF₃; O-methyl; O-ethyl; O—(CH₂)₂—O—CH₃; O—(CH₂)₂—OH; NH₂; NH-methyl; N(methyl)₂; NH-ethyl; N(ethyl)₂; or N(methyl)(ethyl);
m denotes 0, 1 or 2; and
Z denotes C₁₋₄-alkyl, saturated or unsaturated, branched or unbranched, unsubstituted or monosubstituted or polysubstituted with one or more substituents each independently selected from the group consisting of F, Cl, Br, I, OH, ═O, O—C₁₋₄-alkyl, OCF₃, C(═O)—OH and OF₃; phenyl, naphthyl, furyl, pyridyl or thienyl, in each case unsubstituted or monosubstituted or polysubstituted with one or more substituents each independently selected from the group consisting of F, Cl, Br, I, CN, OH, O—C₁₋₄-alkyl, OCF₃, C₁₋₄-alkyl, OF₃, NH₂, NH(C₁₋₄-alkyl), N(C₁₋₄-alkyl)₂, SH, S—C₁₋₈-alkyl, SCF₃, C₁₋₄-alkyl monosubstituted or disubstituted with OH, benzyl and phenyl, wherein benzyl and phenyl can in each case be unsubstituted or monosubstituted or polysubstituted with one or more substituents independently selected from the group consisting of F, Cl, Br, I, CN, OH, O—C₁₋₄-alkyl, OCF₃, C₁₋₄-alkyl, OF₃, NH₂, NH(C₁₋₄-alkyl), N(C₁₋₄-alkyl)₂, SH, S—C₁₋₄-alkyl and SCF₃; C₃₋₁₀-cycloalkyl¹ or heterocyclyl¹, in each case saturated or unsaturated, unsubstituted or monosubstituted or polysubstituted with one or more substituents each independently selected from the group consisting of F, Cl, Br, I, CN, OH, O—C₁₋₄-alkyl, OCF₃, C₁₋₄-alkyl, CF₃, benzyl, phenyl and pyridyl, wherein benzyl, phenyl and pyridyl can in each case be unsubstituted or monosubstituted or polysubstituted with one or more substituents independently selected from the group consisting of F, Cl, Br, I, CN, OH, O—C₁₋₄-alkyl, OCF₃, C₁₋₄-alkyl, CF₃, NH₂, NH(C₁₋₄-alkyl), N(C₁₋₄-alkyl)₂, SH, S—C₁₋₄-alkyl and SCF₃.

5. A compound according to claim 1, wherein R² denotes H; F; Cl; Br; I; CN; NO₂; CF₃; CF₂H; CFH₂; CF₂Cl; CFCl₂; OH; OCF₃; OCF₂H; OCFH₂; OCF₂Cl; OCFCl₂; SH; SCF₃; SCF₂H; SCFH₂; SCF₂Cl; SCFCl₂; C₁₋₁₀-alkyl, saturated or unsaturated, branched or unbranched, unsubstituted or monosubstituted or polysubstituted with one or more substituents each independently selected from the group consisting of F, Cl, Br, I, NO₂, CN, OH, ═O, O—C₁₋₄-alkyl, OCF₃, C(═O)—OH, CF₃, NH₂, NH(C₁₋₄-alkyl), N(C₁₋₄-alkyl)₂, SH, S—C₁₋₄-alkyl, SCF₃ S(═O)₂OH, benzyl, phenyl, pyridyl and thienyl, wherein benzyl, phenyl, pyridyl, thienyl can in each case be unsubstituted or monosubstituted or polysubstituted with one or more substituents independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; C₃₋₁₀-cycloalkyl or heterocyclyl, in each case saturated or unsaturated, unsubstituted or monosubstituted or polysubstituted with one or more substituents independently selected from the group consisting of F, Cl, Br, I, OH, ═O, C₁₋₄-alkyl, O—C₁₋₄-alkyl, OCF₃, C(═O)—OH and CF₃; or C₃₋₁₀-cycloalkyl or heterocyclyl bridged via C₁₋₈-alkyl, in each case saturated or unsaturated, unsubstituted or monosubstituted or polysubstituted with one or more substituents each independently selected from the group consisting of F, Cl, Br, I, OH, ═O, C₁₋₄-alkyl, O—C₁₋₄-alkyl, OCF₃, C(═O)—OH and CF₃, wherein the alkyl chain can in each case be branched or unbranched, saturated or unsaturated, unsubstituted, monosubstituted or polysubstituted with one or more substituents each independently selected from the group consisting of F, Cl, Br, I, OH, ═O and O—C₁₋₄-alkyl; aryl or heteroaryl, in each case unsubstituted or monosubstituted or polysubstituted with one or more substituents each independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₈-alkyl, SCF₃, S(═O)₂OH, benzyl, phenyl, pyridyl and thienyl, wherein benzyl, phenyl, pyridyl, thienyl can in each case be unsubstituted or monosubstituted or polysubstituted with one or more substituents selected independently from one another from the group consisting of F, Cl, Br, I, NO₂, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; or aryl or heteroaryl bridged via C₁₋₈-alkyl, in each case unsubstituted or monosubstituted or polysubstituted with one or more substituents in each case selected independently from one another from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₈-alkyl, SCF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents selected independently from one another from the group consisting of F, Cl, Br, I, NO₂, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH, wherein the alkyl chain can in each case be branched or unbranched, saturated or unsaturated, unsubstituted, monosubstituted or polysubstituted with one or more substituents selected in each case independently of one another from the group consisting of F, Cl, Br, I, OH, ═O and O—C₁₋₄-alkyl.

6. A compound according to claim 1, wherein R⁵, R⁶ and R⁸ each independently denote H; OH; or C₁₋₁₀-alkyl, saturated or unsaturated, branched or unbranched, unsubstituted or monosubstituted or polysubstituted with one or more substituents each independently selected from the group consisting of O—C₁₋₄-alkyl, F, Cl, Br, I and OH.

7. A compound according to claim 1, wherein:
R^{7a} denotes the partial structure (T2) 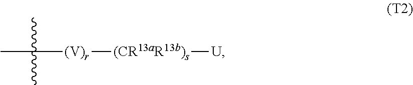
wherein
V denotes C(═O), C(═O)NH, C(═O)—N(C₁₋₁₀-alkyl), or S(═O)₂, or
V denotes NH, N(C₁₋₁₀-alkyl) or NH—S(═O)₂, if T denotes CH;
r denotes 0 or 1;
R^{13a} and R^{13b} each independently denote H; F; Cl; Br; I; NO₂; OF₃; CN; OH; OCF₃; NH₂; C₁₋₄-alkyl, O—C₁₋₄-alkyl, NH—C₁₋₄-alkyl, or N(C₁₋₄-alkyl)₂, in each case saturated or unsaturated, branched or unbranched, unsubstituted or monosubstituted or polysubstituted with one or more substituents each independently selected from the group consisting of F, Cl, Br, I, O—C₁₋₄-alkyl, OH and OCF₃;
with the proviso that if R^{13a} and R^{13b} are bonded to the same carbon atom, only one of the substituents R^{13a} and R^{13b} can denote OH; OCF₃; NH₂; O—C₁₋₄-alkyl, NH—C₁₋₄-alkyl or N(C₁₋₄-alkyl)₂;
s denotes 0, 1, 2, 3 or 4; and
U denotes C₁₋₄-alkyl, saturated or unsaturated, branched or unbranched, unsubstituted or monosubstituted or polysubstituted with one or more substituents each independently selected from the group consisting of F, Cl, Br, I, NO₂, CN, OH, ═O, O—C₁₋₄-alkyl, OCF₃, C(═O)—OH, CF₃, NH₂, NH(C₁₋₄-alkyl), N(C₁₋₄-alkyl)₂, SH, S—C₁₋₄-alkyl, SCF₃ and S(═O)₂OH; C₃₋₁₀-cycloalkyl or heterocyclyl, in each case saturated or unsaturated, unsubstituted or monosubstituted or polysubstituted with one or more substituents each independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents independently selected from the group consisting of F, Cl, Br, I, NO₂, CN, OH, O—C₁₋₄-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH; aryl or heteroaryl, in each case unsubstituted or monosubstituted or polysubstituted with one or more substituents each independently selected from the group consisting of F, Cl, Br, I, NO₂, CN, OH, O—C₁₋₄-alkyl, OCF₃, C₁₋₄-alkyl, C₁₋₄-alkyl monosubstituted or disubstituted with OH, C(═O)—OH, CF₃, NH₂, NH(C₁₋₄-alkyl), N(C₁₋₄-alkyl)₂, SH, S—C₁₋₄-alkyl, SCF₃, S(═O)₂OH, benzyl, phenyl, pyridyl and thienyl, wherein benzyl, phenyl, pyridyl and thienyl can in each case be unsubstituted or monosubstituted or polysubstituted with one or more substituents independently selected from the group consisting of F, Cl, Br, I, NO₂, CN, OH, O—C₁₋₈-alkyl, OCF₃, C₁₋₄-alkyl, C(═O)—OH, CF₃, NH₂, NH(C₁₋₄-alkyl), N(C₁₋₄-alkyl)₂, SH, S—C₁₋₄-alkyl, SCF₃ and S(═O)₂OH.

8. A compound according to claim 1, corresponding to formula (Ie) 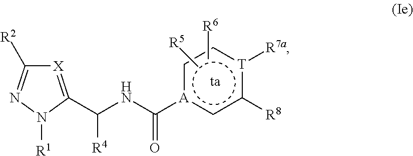
wherein:
X denotes CR³ or N, wherein
R³ denotes H; or C₁₋₁₀-alkyl, saturated or unsaturated, branched or unbranched, unsubstituted;
A denotes N, C or CH;
T denotes N, C or CR^{7b}, wherein
R^{7b} denotes H; F; Cl; Br; I; or OH;
the symbol [symbol] denotes that the non-aromatic ring to can optionally contain at least one unsaturated bond,
with the proviso that if A denotes N, A is not part of the unsaturated bond, and
with the proviso that if T denotes N, then T is not part of the unsaturated bond; R¹ denotes the partial structure (T1-1) 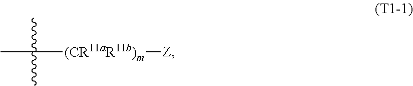
wherein
R^{11a} and R^{11b} each independently denote H; methyl; ethyl; n-propyl; iso-propyl; n-butyl; sec.-butyl; or tert.-butyl;
m denotes 0, 1 or 2;
Z denotes C₁₋₄-alkyl, saturated or unsaturated, branched or unbranched, unsubstituted or monosubstituted or polysubstituted with one or more substituents each independently selected from the group consisting of F, Cl, Br, I, OH, and O—C₁₋₄-alkyl; C₃₋₁₀-cycloalkyl¹, saturated or unsaturated, morpholinyl, piperidinyl, 4-methylpiperazinyl, piperazinyl, in each case unsubstituted or monosubstituted or polysubstituted with one or more substituents each independently selected from the group consisting of F, Cl, Br, I, OH, O—C₁₋₄-alkyl and C₁₋₄-alkyl; phenyl or pyridyl, in each case unsubstituted or monosubstituted or polysubstituted with one or more substituents each independently selected from the group consisting of F, Cl, Br, I, CN, OH, O—C₁₋₄-alkyl, OCF₃, C₁₋₄-alkyl, C₁₋₄-alkyl monosubstituted or disubstituted with OH, CF₃, SH, S—C₁₋₄-alkyl, and SCF₃;
R² denotes H; F; Cl; Br; I; CF₃; CN; methyl; ethyl; n-propyl; iso-propyl; n-butyl; sec.-butyl; tert.-butyl; cyclopropyl; cyclobutyl; or phenyl, in each case unsubstituted or monosubstituted or polysubstituted with one or more substituents independently selected from the group consisting of C₁₋₄-alkyl, O—C₁₋₄-alkyl, F, Cl, Br, I, CF₃ and OCF₃;
R⁴ denotes H;
R⁵, R⁶ and R⁸ each independently denote H or C₁₋₁₀-alkyl, saturated or unsaturated, branched or unbranched, unsubstituted;
R^{7a} denotes the partial structure (T2) 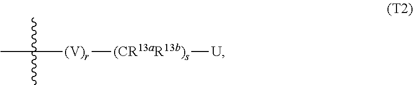
wherein
V denotes C(═O) or S(═O)₂,
r denotes 0 or 1;
R^{13a} and R^{13b} each independently denote H; methyl; ethyl; n-propyl; iso-propyl; n-butyl; sec.-butyl or tert.-butyl;
s denotes 0, 1, 2, 3 or 4; and
U denotes C₁₋₄-alkyl, saturated or unsaturated, branched or unbranched, unsubstituted or monosubstituted or polysubstituted with one or more substituents each independently selected from the group consisting of F, Cl, Br, I, OH, O—C₁₋₄-alkyl, OCF₃, and OF₃; C₃₋₁₀-cycloalkyl, saturated or unsaturated, morpholinyl, piperidinyl, pyrrolidinyl, 4-methylpiperazinyl, piperazinyl, in each case unsubstituted or monosubstituted or polysubstituted with one or more substituents each independently selected from the group consisting of F, Cl, Br, I, CN, OH, O—C₁₋₄-alkyl, OCF₃, C₁₋₄-alkyl and OF₃; phenyl, pyridyl or thienyl, in each case unsubstituted or monosubstituted or polysubstituted with one or more substituents each independently selected from the group consisting of F, Cl, Br, I, CN, OH, O—C₁₋₄-alkyl, OCF₃, C₁₋₄-alkyl monosubstituted or disubstituted with OH, and CF.

9. A compound according to claim 1, selected from the group consisting of:
1 N-((3-tert-butyl-1-(3-chlorophenyl)-1H-pyrazol-5-yl)methyl)-4-(3-(trifluoromethyl)pyridin-2-yl)piperazine-1-carboxamide;
2 N-((3-tert-butyl-1-(3-chlorophenyl)-1H-pyrazol-5-yl)methyl)-4-(3-chloropyridin-2-yl)piperazine-1-carboxamide;
3 N-((1-(3-chlorophenyl)-3-(trifluoromethyl)-1H-pyrazol-5-yl)methyl)-4-(3-chloropyridin-2-yl)piperazine-1-carboxamide;
4 N-((1-(4-chlorophenyl)-3-(trifluoromethyl)-1H-pyrazol-5-yl)methyl)-4-(3-chloropyridin-2-yl)piperazine-1-carboxamide;
5 N-((3-tert-butyl-1-(3-chlorophenyl)-1H-pyrazol-5-yl)methyl)-4-(2-fluorophenyl)piperazine-1-carboxamide;
6 N-((3-tert-butyl-1-(3-chloro-4-fluorophenyl)-1H-pyrazol-5-yl)methyl)-4-(3-chloropyridin-2-yl)piperazine-1-carboxamide;
7 N-((3-tert-butyl-1-(pyridin-2-yl)-1H-pyrazol-5-yl)methyl)-4-(3-chloropyridin-2-yl)piperazine-1-carboxamide;
8 4-(3-chloropyridin-2-yl)-N-((1-m-tolyl-3-(trifluoromethyl)-1H-pyrazol-5-yl)methyl)piperazine-1-carboxamide;
9 N-((1-(3-chlorophenyl)-4-methyl-3-(trifluoromethyl)-1H-pyrazol-5-yl)methyl)-4-(3-chloropyridin-2-yl)piperazine-1-carboxamide;
10 N-((1-(3-chlorophenyl)-3-cyclopropyl-1H-pyrazol-5-yl)methyl)-4-(3-chloropyridin-2-yl)piperazine-1-carboxamide;
11 4-(3-chloropyridin-2-yl)-N-((1-(4-methoxybenzyl)-3-(trifluoromethyl)-1H-pyrazol-5-yl)methyl)piperazine-1-carboxamide;
12 4-(3-chloropyridin-2-yl)-N-((1-pentyl-3-(trifluoromethyl)-1H-pyrazol-5-yl)methyl)piperazine-1-carboxamide;
13 4-(3-chloropyridin-2-yl)-N-((1-(tetrahydro-2H-pyran-4-yl)-3-(trifluoromethyl)-1H-pyrazol-5-yl)methyl)piperazine-1-carboxamide;
14 N-((1-(3-chlorophenyl)-3-(trifluoromethyl)-1H-pyrazol-5-yl)methyl)-4-methylpiperazine-1-carboxamide;
15 N-((1-(3-chlorophenyl)-3-(trifluoromethyl)-1H-pyrazol-5-yl)methyl)-4-ethylpiperazine-1-carboxamide;
16 4-tert-butyl-N-((1-(3-chlorophenyl)-3-(trifluoromethyl)-1H-pyrazol-5-yl)methyl)piperazine-1-carboxamide;
17 N-((1-(3-chlorophenyl)-3-(trifluoromethyl)-1H-pyrazol-5-yl)methyl)-4-cyclohexylpiperazine-1-carboxamide;
18 N-((1-(3-chlorophenyl)-3-(trifluoromethyl)-1H-pyrazol-5-yl)methyl)-4-(thiophen-2-yl)piperazine-1-carboxamide;
19 N-((1-(3-chlorophenyl)-3-(trifluoromethyl)-1H-pyrazol-5-yl)methyl)-4-phenylpiperazine-1-carboxamide;
20 4-benzyl-N-((3-tert-butyl-1-(3-chlorophenyl)-1H-pyrazol-5-yl)methyl)piperazine-1-carboxamide;
21 N-((3-tert-butyl-1-(3-chlorophenyl)-1H-pyrazol-5-yl)methyl)-4-(1-phenylethyl)piperazine-1-carboxamide;
22 N-((1-(3-chlorophenyl)-3-(trifluoromethyl)-1H-pyrazol-5-yl)methyl)-4-(1-(4-fluorophenyl)ethyl)piperazine-1-carboxamide;
23 N-((1-(3-chlorophenyl)-3-(trifluoromethyl)-1H-pyrazol-5-yl)methyl)-4-(methylsulfonyl)piperazine-1-carboxamide;
24 4-acetyl-N-((1-(3-chlorophenyl)-3-(trifluoromethyl)-1H-pyrazol-5-yl)methyl)piperazine-1-carboxamide;
25 4-benzoyl-N-((1-(3-chlorophenyl)-3-(trifluoromethyl)-1H-pyrazol-5-yl)methyl)piperazine-1-carboxamide;
26 N-((1-(3-chlorophenyl)-3-(trifluoromethyl)-1H-pyrazol-5-yl)methyl)-4-phenylpiperidin-1-carboxamide;
27 N-((1-(3-chlorophenyl)-3-(trifluoromethyl)-1H-pyrazol-5-yl)methyl)-4-(2-methoxyphenyl)piperidine-1-carboxamide;
28 N-((1-(3-chlorophenyl)-3-(trifluoromethyl)-1H-pyrazol-5-yl)methyl)-4-(2,4-difluorophenyl)piperidine-1-carboxamide;
29 N-((1-(3-chlorophenyl)-3-(trifluoromethyl)-1H-pyrazol-5-yl)methyl)-4-hydroxy-4-phenylpiperidine-1-carboxamide;
30 N-((1-(3-chlorophenyl)-3-(trifluoromethyl)-1H-pyrazol-5-yl)methyl)-1-methylpiperidine-4-carboxamide;
31 1-acetyl-N-((1-(3-chlorophenyl)-3-(trifluoromethyl)-1H-pyrazol-5-yl)methyl)piperidine-4-carboxamide;
32 1-benzoyl-N-((1-(3-chlorophenyl)-3-(trifluoromethyl)-1H-pyrazol-5-yl)methyl)piperidine-4-carboxamide;
33 N-((1-(3-chlorophenyl)-3-(trifluoromethyl)-1H-pyrazol-5-yl)methyl)-4-isopropylcyclohexane carboxamide;
34 N-((1-(3-chlorophenyl)-3-(trifluoromethyl)-1H-pyrazol-5-yl)methyl)-4-(4-fluorophenyl)-5,6-dihydropyridine-1(2H)-carboxamide;
35 N-((1-(3-chlorophenyl)-3-(trifluoromethyl)-1H-pyrazol-5-yl)methyl)-4-hydroxycyclohex-1-ene carboxamide;
36 N-((1-(3-chlorophenyl)-3-(trifluoromethyl)-1H-pyrazol-5-yl)methyl)-1-ethyl-1,2,3,6-tetrahydropyridine-4-carboxamide;
37 N-((1-(3-chlorophenyl)-3-(trifluoromethyl)-1H-pyrazol-5-yl)methyl)-1-(4-fluorophenylsulfonyl)-1,2,3,6-tetrahydropyridine-4-carboxamide;
38 N-((1-(3-chlorophenyl)-3-(trifluoromethyl)-1H-pyrazol-5-yl)methyl)-4-ethylcyclohex-3-ene carboxamide;
39 (S)-4-(3-chlor-5-(1,2-dihydroxyethyl)pyridin-2-yl)-N-((1-(3-chlorophenyl)-3-(trifluoromethyl)-1H-pyrazol-5-yl)methyl)piperazine-1-carboxamide;
40 (S)-4-(3-chlor-5-(1,2-dihydroxyethyl)pyridin-2-yl)-N-((1-(3-chlorophenyl)-3-(trifluoromethyl)-1H-pyrazol-5-yl)methyl)-5,6-dihydropyridine-1(2H)-carboxamide;
41 (S)-4-(3-chlor-5-(1,2-dihydroxyethyl)pyridin-2-yl)-N-((1-(3-chlorophenyl)-3-(trifluoromethyl)-1H-pyrazol-5-yl)methyl)-4-fluoropiperidine-1-carboxamide;
42 N-((1-(3-chlorophenyl)-3-(trifluoromethyl)-1H-pyrazol-5-yl)methyl)-1-(3-chloropyridin-2-yl)-1,2,3,6-tetrahydropyridine-4-carboxamide;
43 N-((3-tert-butyl-1-(3-chlorophenyl)-1H-1,2,4-triazol-5-yl)methyl)-4-(3-chloropyridin-2-yl)piperazine-1-carboxamide;
44 N-((3-tert-butyl-1-(3-chlorophenyl)-1H-1,2,4-triazol-5-yl)methyl)-4-(1-(4-fluorophenyl)ethyl)piperazine-1-carboxamide;
45 4-(1-(4-fluorophenyl)ethyl)-N-((1-hexyl-3-(trifluoromethyl)-1H-1,2,4-triazol-5-yl)methyl)piperazine-1-carboxamide;
46 N-((1-(3-chlorophenyl)-3-(trifluoromethyl)-1H-pyrazol-5-yl)methyl)-4-(2-fluorophenyl)piperazine-1-carboxamide;
47 N-((1-(3-chlorophenyl)-3-(trifluoromethyl)-1H-pyrazol-5-yl)methyl)-4-(4-fluorophenyl)piperazine-1-carboxamide;
48 N-((1-(3-chlorophenyl)-3-(trifluoromethyl)-1H-pyrazol-5-yl)methyl)-4-(2-methoxyphenyl)piperazine-1-carboxamide;
49 N-((1-(3-chlorophenyl)-3-(trifluoromethyl)-1H-pyrazol-5-yl)methyl)-4-(3-methoxyphenyl)piperazine-1-carboxamide;
50 N-((1-(3-chlorophenyl)-3-(trifluoromethyl)-1H-pyrazol-5-yl)methyl)-4-(4-methoxyphenyl)piperazine-1-carboxamide;
51 4-(2-chlorophenyl)-N-((1-(3-chlorophenyl)-3-(trifluoromethyl)-1H-pyrazol-5-yl)methyl)piperazine-1-carboxamide;
52 N-((3-tert-butyl-1-(3-chlorophenyl)-1H-pyrazol-5-yl)methyl)-4-(3-chlorophenyl)piperazine-1-carboxamide;
53 N-((3-tert-butyl-1-(3-chlorophenyl)-1H-pyrazol-5-yl)methyl)-4-(4-chlorophenyl)piperazine-1-carboxamide;
54 4-(4-chlorophenyl)-N-((1-(3-chlorophenyl)-3-(trifluoromethyl)-1H-pyrazol-5-yl)methyl)piperazine-1-carboxamide;
55 N-((3-tert-butyl-1-(3-chlorophenyl)-1H-pyrazol-5-yl)methyl)-4-(3-(trifluoromethyl)pyridin-2-yl)piperazine-1-carboxamide; and
56 N-((1-(3-chlorophenyl)-3-(trifluoromethyl)-1H-pyrazol-5-yl)methyl)-4-(3-(trifluoromethyl)pyridin-2-yl)piperazine-1-carboxamide;
in each case in the form of a free compound; a racemate; an isolated enantiomer or diastereomer, a mixture of enantiomers or diastereomers; or in the form of a salts of a physiologically compatible acid or base.

10. A pharmaceutical composition comprising a compound according to claim 1 and at least one pharmaceutically acceptable additive, auxiliary substance or further active compound.

11. A process for preparing a compound according to claim 1, said process comprising:
converting a compound corresponding to formula (II) 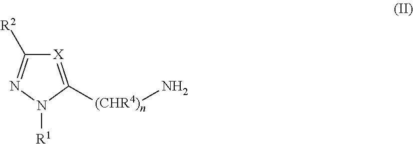
wherein X, R¹, R², R⁴ and n have the meanings given in claim 1, to a compound corresponding to formula (V) 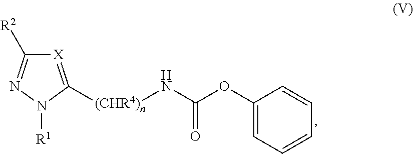
wherein X, R¹, R², R⁴, have the meanings given in claim 1, in a reaction medium, in the presence of phenyl chloroformate, optionally in the presence of at least one base and/or one coupling reagent, and this is optionally purified and/or isolated, and
reacting the compound corresponding to formula (V) with a compound corresponding to formula (VI), 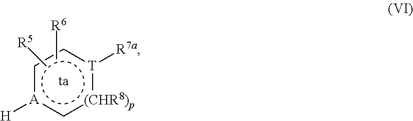
wherein R⁵, R⁶, R^{7a}, R⁸, p and T have the meanings given in claim 1, and A denotes N, in a reaction medium, optionally in the presence of at least one suitable coupling reagent, optionally in the presence of at least one base, to form a compound corresponding to formula (I) 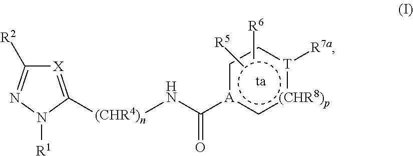
wherein X, R¹, R², R⁴, R⁵, R⁶, R^{7a}, R⁸, n, p and T as well as in [symbol] have the meanings given in claim 1, and A denotes N;
or said process comprising:
reacting a compound corresponding to formula (II) 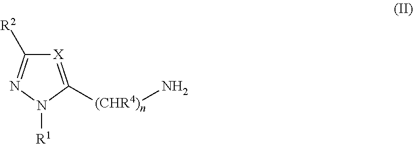
wherein X, R¹, R², R⁴ and n have the meanings given in claim 1, in a reaction medium, optionally in the presence of at least one suitable coupling reagent, optionally in the presence of at least one base, with a compound corresponding to formula (III) or formula (IV) 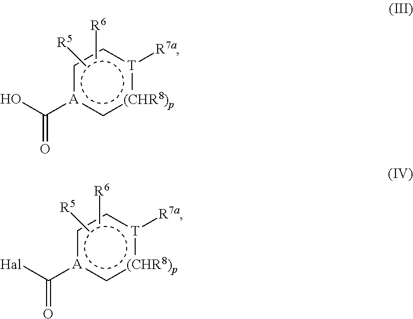
wherein Hal denotes a halogen, and R⁵, R⁶, R^{7a}, R⁸, p, [symbol] and T in each case have the meanings given in claim 1, and A denotes CH or C in a reaction medium, optionally in the presence of at least one suitable one coupling reagent, optionally in the presence of at least one base, to form a compound corresponding to formula (I), 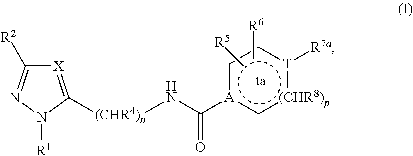
wherein X, R¹, R², R⁴, R⁵, R⁶, R^{7a}, R⁸, n, p, [symbol] and T have the meanings given in claim 1, and A denotes CH or C.

12. A method of treating a disorder or disease state selected from the group consisting of pain; hyperalgesia; allodynia; causalgia; migraine; depression; neuropathy; nerve damage; neurodegenerative diseases; cognitive dysfunctions; epilepsy; respiratory pathway diseases; coughing; urinary incontinence; overactive bladder; diseases and/or injuries of the gastrointestinal tract; duodenal ulcers; gastric ulcers; irritable bowel syndrome; strokes; occular irritation; skin irritation; neurotic skin diseases; allergic skin diseases; psoriasis; leukodermia; Herpes simplex; inflammation; diarrhoea; pruritus; osteoporosis; arthritis; osteoarthritis; rheumatic diseases; eating disorders; medicament dependence; medicament misuse; withdrawal symptoms in medicament dependence; development of tolerance to medicaments; drug dependence; drug misuse; withdrawal symptoms in drug dependence; alcohol dependence; alcohol misuse and withdrawal symptoms in alcohol dependence; diuresis; antinatriuresis; to influence the cardiovascular system; to improve vigilance; to treat wounds and/or burns; to treat severed nerves; to increase libido; to modulate movement activity; for anxiolysis; for local anaesthesia and/or to inhibit undesired side effects triggered by the administration of vanilloid receptor 1 agonists, in a subject in need thereof, said method comprising administering to said subject a pharmaceutically effective amount of a compound according to claim 1.

13. A method according to claim 12, wherein said disorder is pain selected from the group consisting of acute pain, chronic pain, neuropathic pain, visceral pain and arthritic pain.

14. A method according to claim 12, wherein said disorder is a neurodegenerative disease, selected from the group consisting of multiple sclerosis, Alzheimer's disease, Parkinson's disease, and Huntington's chorea; a memory disorder; a respiratory pathway disease selected from the group consisting of asthma, bronchitis, inflammation of the lungs and pneumonia; inflammation of the stomach, eyes, bladder, skin or nasal mucosa; an eating disorder selected from the group consisting of bulimia, cachexia, anorexia and obesity;
development of a tolerance to natural or synthetic opioids; or to inhibit undesired side effects selected from the group consisting of hyperthermia, hypertension and bronchial constriction triggered by administration of a vanilloid receptor 1 agonist selected from the group consisting of capsaicin, resiniferatoxin, olvanil, arvanil, nuvanil and capsavanil.
